(19) United States
(12) Patent Application Publication
(10) Pub. No.: US 2021/0396762 A1
(43) Pub. Date: Dec. 23, 2021

(54) METHODS FOR PEPTIDE ANALYSIS EMPLOYING MULTI-COMPONENT DETECTION AGENT AND RELATED KITS

(71) Applicant: Encodia, Inc., San Diego, CA (US)

(72) Inventors: Mark S. CHEE, San Diego, CA (US); Michael Phillip WEINER, San Diego, CA (US)

(73) Assignee: Encodia, Inc., San Diego, CA (US)

(21) Appl. No.: 17/348,643

(22) Filed: Jun. 15, 2021

Related U.S. Application Data
(60) Provisional application No. 63/041,777, filed on Jun. 19, 2020.

(51) Int. Cl. G01N 33/68; G01N 33/58

(52) U.S. Cl. CPC G01N 33/6803; G01N 33/581; G01N 33/582

(57) ABSTRACT

The present disclosure relates to methods and kits for analysis of peptides, polypeptides and proteins, employing a multi-component detection agent(s). In some embodiments, the method is useful for identifying the terminal amino acid of the peptide. In some embodiments, the multi-component detection agent includes a first detection agent and second detection agent which, when in proximity, is capable of generating a detectable signal.

### CROSS-REFERENCE TO RELATED APPLICATION

The present application claims priority to U.S. provisional patent application No. 63/041,777, filed on Jun. 19, 2020, the disclosure and content of which is incorporated herein by reference in its entirety for all purposes.

### SEQUENCE LISTING ON ASCII TEXT

This patent application file contains a Sequence Listing submitted in computer readable ASCII text format (file name: 4614-2002400_SeqList, generated on Jun. 11, 2021; size: 8573 bytes). The content of the Sequence Listing file is incorporated herein by reference in its entirety.

### TECHNICAL FIELD

This disclosure generally relates to methods and kits for analysis of macromolecules, including peptides, polypeptides and proteins, employing a multi-component detection agent(s). In some embodiments, the method is useful for identifying the terminal amino acid of the peptide. In some embodiments, the multi-component detection agent includes a first detection agent and second detection agent which, when in sufficient proximity, generates a detectable label which is capable of generating a detectable signal.

### BACKGROUND

Proteomics is the study of the structure and function of proteins in biological systems and encompasses a wide range of applications, including protein expression profiling in healthy versus diseased states of an organism, analyzing the interaction of proteins in living organisms, and mapping of protein modifications and identification of how, when and where proteins are modified within a living cell. Despite significant advances, there remains a need in the art for improved techniques for the identification and quantification of proteins in biological samples. For example, although high-throughput techniques have been developed for sequencing and/or analyzing DNA and RNA within a biological sample, such advances are still needed at the protein level.

Traditionally, mass spectrometry (MS) has been employed for proteomic characterization. However, MS suffers from a number of drawbacks, including the requirement for relatively large sample sizes and limitations associated with quantification and dynamic range. For example, since proteins ionize at different levels of efficiencies, relative amounts are difficult to compare between samples. Also, concentrations of proteins within samples can vary over a very large range, making characterization of the same very difficult. Further complicating MS analysis is the frequent loss of phosphate upon ionization, which limits the analysis of phosphopeptides.

More recently, advances have been made in the field of digital analysis of proteins by end sequencing (referred to as DAPES) as disclosed, for example, by Mitra and Tessler in PCT Publication No. WO2010/065531. In this method, surface bound peptides are directly sequenced using a modified Edman degradation step followed by detection, such as with a labeled antibody. More specifically, the N-terminal amino acid of an immobilized protein is first reacted with phenylisothiocyanate (PITC) to form a phenylthiocarbamoyl derivative (PTC-derivative). A labeled antibody which binds both the phenyl group of the PTC-derivative and the side chain of the N-terminal amino acid is then used for detection. After detection of the bound antibody, the antibody is stripped and the procedure repeated with antibodies that will detect other PTC-derivatives (i.e., other N-terminal amino acids). By repeating the above binding, detection and stripping steps using 20 unique antibodies that recognize each of the 20 PTC-derivatives (one for each of the 20 naturally occurring amino acids), the identity of all the N-terminal amino acids of the immobilized protein can be determined. The terminal amino acids of the immobilized proteins are then removed, and the procedure repeated for the newly exposed N-terminal amino acids.

A modification of DAPES was disclosed by Havranek and Borgo in Published PCT Publication No. WO2014/0273004. In this method, single molecule sequencing of peptides is achieved by contacting the peptide with one or more fluorescently labelled N-terminal amino acid binding proteins (NAABs), detecting the fluorescence of a NAAB bound to the N-terminal amino acid, identifying the N-terminal amino acid based on the fluorescence detected, removing the NAAB from the peptide, and repeating with NAABs that bind to different N-terminal amino acids. Following such steps, the N-terminal amino acid is cleaved from the polypeptide by Edman degradation, and the procedure repeated for the newly-exposed N-terminal amino acids.

In another method, as disclosed by Cargille and Stephenson in PCT Publication No. WO2010/065322, sequencing of polypeptide is accomplished by use of labelled N-terminal amino acids complexing agents, followed by Edman degradation or aminopeptidase cleavage cycles. Other techniques for characterizing proteins include those disclosed by Kwagh et al. in U.S. Patent Application Publication No. US2003/0138831, by Marcotte et al. in U.S. Patent Application Publication No. US2014/0349860, and by Hessellberth in PCT Publication No. WO2013/112745.

However, such existing techniques suffer from a number of limitations, particularly in the context of single molecule detection, including low signal-to-noise ratios, lacking the ability to control the binding reaction, as well as non-specific binding to the substrate (e.g., high background fluorescence). Despite the advances that have been made in this field, there remains a significant need for improved techniques relating to peptide sequencing and/or analysis, as well as to products, methods and kits for accomplishing the same. The present disclosure fulfills these and other needs, as evident in reference to the following disclosure.

These and other aspects of the invention will be apparent upon reference to the following detailed description. To this end, various references are set forth herein which describe in more detail certain background information, procedures, compounds and/or compositions, and are each hereby incorporated by reference in their entireties.

### BRIEF SUMMARY

The summary is not intended to be used to limit the scope of the claimed subject matter. Other features, details, utilities, and advantages of the claimed subject matter will be apparent from the detailed description including those aspects disclosed in the accompanying drawings and in the appended claims.

Provided is a method for analyzing a polypeptide, comprising the steps of: (a) providing a polypeptide and an associated first detection agent attached to a solid support; (b) contacting the polypeptide with a binding agent capable of binding to the polypeptide, wherein the binding agent is associated with a second detection agent, whereby binding between the polypeptide and the binding agent brings the first detection agent and the second detection agent into sufficient proximity to interact with each other and generate a detectable label; and (c) detecting a signal generated by the detectable label; and repeating step (b) and step (c) sequentially one or more times. In some embodiments, analyzing the polypeptide comprises identifying at least a portion of an amino acid sequence of the polypeptide, for example, the N-terminal amino acid (NTAA) residue of the polypeptide. In some embodiments, the method is performed on a plurality of polypeptides. In some embodiments, in the step (b), the method comprises contacting the polypeptide with a plurality of binding agents as a mixture. In some embodiments, each binding agent is associated with a different second detection agent; and the signal generated by the detectable label is different for each binding agent. In some embodiments, the method further comprises: (d) removing a portion of the polypeptide, wherein step (d) is performed after step (c) and before repeating step (b), and wherein steps (b)-(d) are repeated sequentially one or more times.

Also provided herein is a method of identifying one or more binding events between a plurality of binding agents and a plurality of polypeptides, comprising: (a) providing a plurality of polypeptides attached to a solid support, wherein each polypeptide from the plurality of polypeptides is associated with a first detection agent; (b) contacting a polypeptide from the plurality of polypeptides with a plurality of binding agents, wherein at least one binding agent from the plurality of binding agents is capable of binding to the polypeptide, and wherein each binding agent from the plurality of binding agents is associated with a second detection agent, whereby binding between the polypeptide and the at least one binding agent brings the first detection agent and the second detection agent into sufficient proximity to interact with each other and generate a detectable label; (c) detecting a signal generated by the detectable label, thereby identifying the binding between the polypeptide and the at least one binding agent; (d) optionally, removing a portion of the polypeptide; and repeating steps (b), (c) and (d) sequentially one or more times.

Also provided is a kit including a support; a first detection agent configured to be associated with a polypeptide, directly or indirectly, joined to a support; a binding agent capable of binding to the polypeptide, wherein the binding agent is associated with a second detection agent, wherein binding between the polypeptide and the binding agent brings the first detection agent and the second detection agent into sufficient proximity to generate a detectable label; and optionally a reagent for modifying a terminal amino acid of the polypeptide and/or a reagent for removing a portion of the polypeptide

### BRIEF DESCRIPTION OF THE DRAWINGS

Non-limiting embodiments of the present invention will be described by way of example with reference to the accompanying figures, which are schematic and are not intended to be drawn to scale. For purposes of illustration, not every component is labeled in every figure, nor is every component of each embodiment of the invention shown where illustration is not necessary to allow those of ordinary skill in the art to understand the invention.

FIG. 1A illustrates various motifs (designated A, B, C and D) for providing the peptide 112 and first detection agent 120 joined to the solid support 110, optionally using a linker 114 and linker 122. In FIG. 1B, a cognate binding agent 200 is shown selectively binding to NTAA 210 of peptide 112. Cognate binding agent 200 is linked to first detection agent 204 through linker 216. Such selective binding of the cognate binding agent to the NTAA brings first detection agent 120 and second detection agent 204 into sufficient proximity, which generates a detectable signal. In FIG. 1C, when the peptide is contacted with non-cognate binding agent 202, which moiety is not capable of selectively binding NTAA 210 of peptide 112, the first detection agent 120 and second detection agent 204 are not in proximity, and thus no signal is generated.

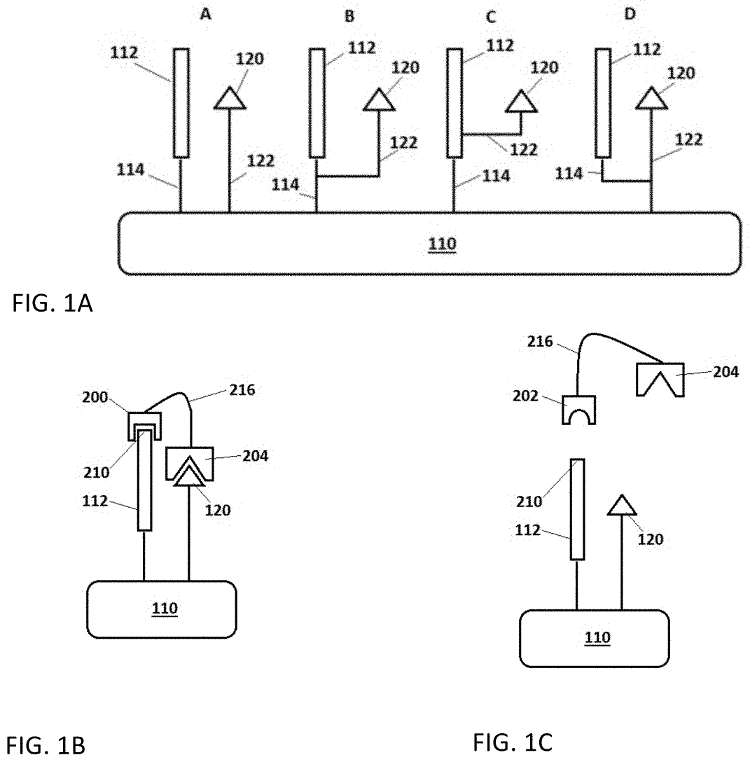

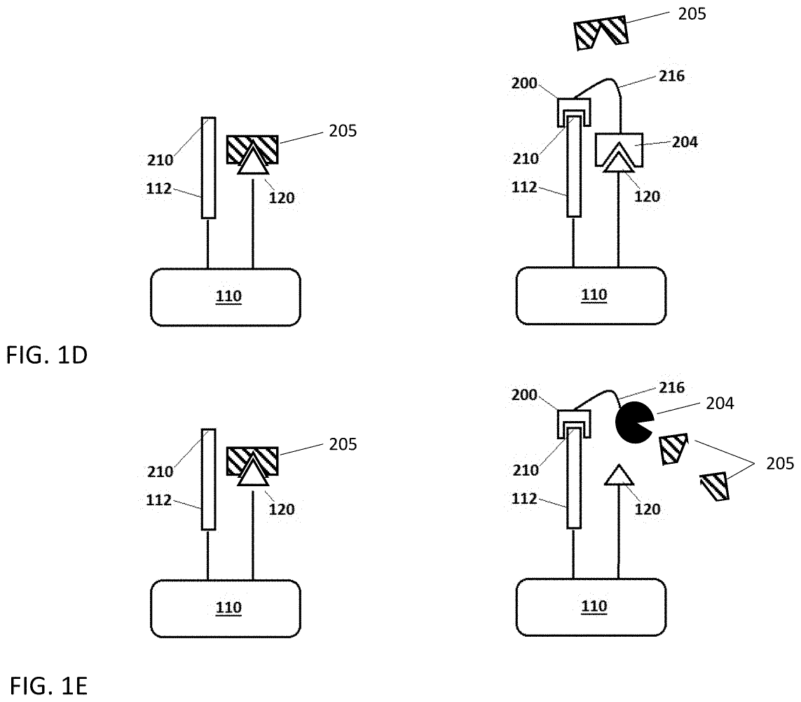

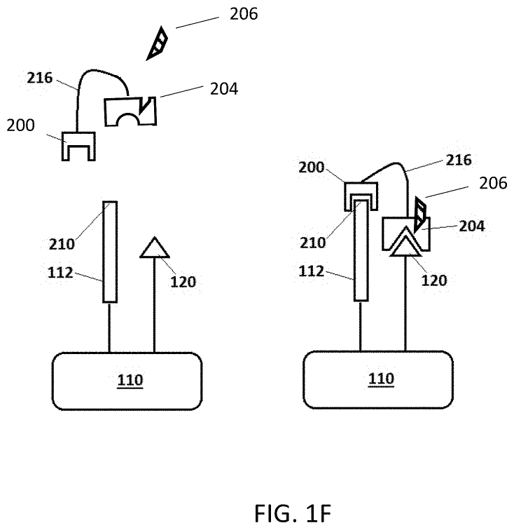

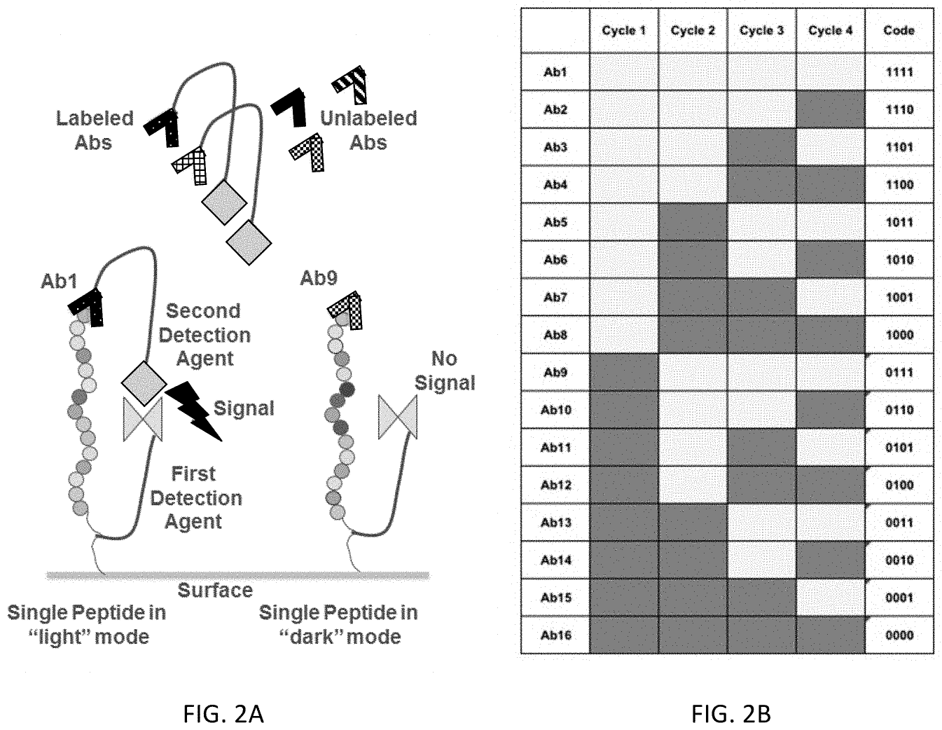

In FIG. 1D, on the left side, a blocking molecule 205 is shown binding to the first detection agent 120, and no detectable signal is generated when the first detection agent is blocked. On the right side of FIG. 1D, the blocking molecule 205 is displaced or removed when the cognate binding agent 200 selectively binds to NTAA 210 of peptide 112. Such selective binding of the cognate binding agent to the NTAA brings first detection agent 120 and second detection agent 204 into sufficiently sufficient proximity, displacing the blocking molecule, which generates a detectable signal.

In FIG. 1E, on the left side, a blocking molecule 205 is shown binding to the first detection agent 120, and no detectable signal is generated when the first detection agent is blocked. On the right side of FIG. 1E, the blocking molecule 205 is removed when the cognate binding agent 200 selectively binds to NTAA 210 of peptide 112. Such selective binding of the cognate binding agent to the NTAA brings second detection agent 204 in sufficient proximity to cleave the blocking molecule 205, allowing the first detection agent 120 to generate a detectable signal without inhibition.

FIG. 1F, a cognate binding agent 200 is linked to second detection agent 204 through linker 216. The second detection agent 204 requires allosteric activation by an activating molecule 206 to change conformation to allow interaction with first detection agent 120. On the right side of FIG. 1F, binding of the cognate binding agent to the NTAA and binding of the activating agent 206 to the second detection agent 204 allows the first detection agent 120 to be in sufficient proximity to second detection agent 204, generating a detectable signal.

FIG. 2A illustrates a decoding technique for identification of N-terminal amino acids (NTAAs) of a polypeptide through with repeated cycles of binding pools of cognate binding agents. For example, the NTAA on the left is selectively bound by a cognate binding agent, and the first and second detection agents are in signal-generating proximity (“light” mode), while an unlabeled antibody on the right selectively binding the NTAA but does not generate a signal (the “dark” mode). FIG. 2B illustrates an exemplary resulting digital readout using various labeled and unlabeled binding agents through multiple cycles of binding.

### DETAILED DESCRIPTION

Provided herein are methods and kits for analyzing a polypeptide, including providing a polypeptide and an associated first detection agent joined to a support; contacting the polypeptide with a binding agent capable of binding to the polypeptide, wherein the binding agent is associated with a second detection agent, whereby binding between the polypeptide and the binding agent brings the first detection agent and the second detection agent into sufficient proximity to interact with each other and generate a detectable label; and detecting a signal generated by the detectable label. In some embodiments, the contacting of the polypeptide with a binding agent (associated with a second detection agent) capable of binding to the polypeptide and detecting the signal generated by the detectable label is repeated sequentially one or more times. Also provided are kits containing components and/or reagents for performing the provided methods. In some embodiments, the kits also include instructions for preparing the components and performing any of the methods provided for peptide analysis.

Recognition and binding of immobilized molecular targets using binding agents can be useful for characterization and/or detection of biomolecules such as peptides. Labeled antibodies with a detectable label have been used to detect N-terminal amino acids (PCT Publication No. WO2010/065531). In one example, single molecule sequencing of peptides is achieved by contacting an immobilized peptide with one or more fluorescently labelled N-terminal amino acid binding proteins (NAABs), detecting the fluorescence of a NAAB bound to the N-terminal amino acid, identifying the N-terminal amino acid based on the fluorescence detected, removing the NAAB from the peptide, and repeating with NAABs that bind to different N-terminal amino acids (PCT Publication No. WO2014/0273004). Following such steps, the N-terminal amino acid is cleaved from the polypeptide by Edman degradation, and the procedure repeated for the newly-exposed N-terminal amino acids. In another example, sequencing of polypeptide is accomplished by use of labelled N-terminal amino acids complexing agents, followed by Edman degradation or aminopeptidase cleavage cycles (PCT Publication No. WO2010/065322). Other techniques for characterizing proteins include those described in U.S. Patent Application Publication No. US2003/0138831, US2014/0349860, and PCT Publication No. WO2013/112745.

However, current reagents and techniques are somewhat limited particularly in the context of detection of a single molecule immobilized on a solid support, including low signal-to-noise ratios, lacking the ability to control the binding reaction, as well as non-specific binding to the support (e.g., high background fluorescence). Accordingly, there remains a need for improved techniques relating to analyzing peptides, as well as to products, methods and kits for accomplishing the same.

The present invention provides novel methods and compositions which may be utilized in a wide variety of binding agent-based assays, and further provides other related advantages. For example, the use of a two-component detection system and the detectable signal generated by the provided methods allows for signal amplification and other advantages. In preferred embodiments, signal can be generated only when the first detection agent and the second detection agent are in sufficient proximity; this solves the problem of unspecific attachment of the binding agent to the solid support that would result in a background signal. Having the disclosed split components, no such signal is generated unless the cognate binding agent recognizes the polypeptide and brings the first and the second detection agents into sufficient proximity to generate a detectable label. In one example, the two-component detection agent comprises a split detection agent, e.g., a split protein. Split proteins have been used for the detection and/or quantification of protein interactions, such as protein-fragment complementation assays (Michnick et al., Nat Rev Drug Discov 6, 569-82 (2007); Remy & Michnick, Methods Mol Biol 1278, 467-81 (2015); U.S. Patent Application Publication No. US 2008/0248463), split protein complementation (Shekhawat & Ghosh, Curr Opin Chem Biol 15, 789-97 (2011)), or bimolecular fluorescence complementation (Miller et al., J Mol Biol 427, 2039-55 (2015); Kerppola, T. K., Chem Soc Rev 38, 2876-2886 (2009)). The present disclosure provides, in part, use of multi-component detection agents in or with a method for highly-parallel, high throughput digital macromolecule (e.g., polypeptide) characterization and quantitation, with direct applications to protein and peptide characterization and sequencing. In some embodiments, the analysis is applicable to macromolecules, e.g., a plurality of macromolecules obtained from a sample, such as a plurality of peptides and proteins. In some embodiments, the sample is obtained from a subject and comprises unknown polypeptides.

Numerous specific details are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described. They instead can be applied, alone or in some combination, to one or more of the other embodiments of the disclosure, whether or not such embodiments are described, and whether or not such features are presented as being a part of a described embodiment. For the purpose of clarity, technical material that is known in the technical fields related to the claimed subject matter has not been described in detail so that the claimed subject matter is not unnecessarily obscured.

All publications, including patent documents, scientific articles and databases, referred to in this application are incorporated by reference in their entireties for all purposes to the same extent as if each individual publication were individually incorporated by reference. Citation of the publications or documents is not intended as an admission that any of them is pertinent prior art, nor does it constitute any admission as to the contents or date of these publications or documents.

All headings are for the convenience of the reader and should not be used to limit the meaning of the text that follows the heading, unless so specified.

### Definitions

Unless defined otherwise, all technical and scientific terms used herein have the same meaning as is commonly understood by one of ordinary skill in the art to which the present disclosure belongs. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a,” “an” and “the” include plural referents unless the context clearly dictates otherwise. Thus, for example, reference to “a peptide” includes one or more peptides, or mixtures of peptides. Also, and unless specifically stated or obvious from context, as used herein, the term “or” is understood to be inclusive and covers both “or” and “and”.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X.”

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

An “individual” or “subject” includes a mammal. Mammals include, but are not limited to, domesticated animals (e.g., cows, sheep, cats, dogs, and horses), primates (e.g., humans and non-human primates such as monkeys), rabbits, and rodents (e.g., mice and rats). An “individual” or “subject” may include birds such as chickens, vertebrates such as fish and mammals such as mice, rats, rabbits, cats, dogs, pigs, cows, ox, sheep, goats, horses, monkeys and other non-human primates. In certain embodiments, the individual or subject is a human.

As used herein, the term “sample” refers to anything which may contain an analyte for which an analyte assay is desired. As used herein, a “sample” can be a solution, a suspension, liquid, powder, a paste, aqueous, non-aqueous or any combination thereof. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

In some embodiments, the sample is a biological sample. A biological sample of the present disclosure encompasses a sample in the form of a solution, a suspension, a liquid, a powder, a paste, an aqueous sample, or a non-aqueous sample. As used herein, a “biological sample” includes any sample obtained from a living or viral (or prion) source or other source of macromolecules and biomolecules, and includes any cell type or tissue of a subject from which nucleic acid, protein and/or other macromolecule can be obtained. The biological sample can be a sample obtained directly from a biological source or a sample that is processed. For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants and processed samples derived therefrom. In some embodiments, the sample can be derived from a tissue or a body fluid, for example, a connective, epithelium, muscle or nerve tissue; a tissue selected from the group consisting of brain, lung, liver, spleen, bone marrow, thymus, heart, lymph, blood, bone, cartilage, pancreas, kidney, gall bladder, stomach, intestine, testis, ovary, uterus, rectum, nervous system, gland, and internal blood vessels; or a body fluid selected from the group consisting of blood, urine, saliva, bone marrow, sperm, an ascitic fluid, and subfractions thereof, e.g., serum or plasma.

As used herein, the term “macromolecule” encompasses large molecules composed of smaller subunits. Examples of macromolecules include, but are not limited to peptides, polypeptides, proteins, nucleic acids, carbohydrates, lipids, macrocycles, or a combination or complex thereof. A macromolecule also includes a chimeric macromolecule composed of a combination of two or more types of macromolecules, covalently linked together (e.g., a peptide linked to a nucleic acid). A macromolecule may also include a “macromolecule assembly”, which is composed of non-covalent complexes of two or more macromolecules.

As used herein, the term “polypeptide” encompasses peptides and proteins, and refers to a molecule comprising a chain of two or more amino acids joined by peptide bonds. In some embodiments, a polypeptide comprises 2 to 50 amino acids, e.g., having more than 20-30 amino acids. In some embodiments, a peptide does not comprise a secondary, tertiary, or higher structure. In some embodiments, the polypeptide is a protein. In some embodiments, a protein comprises 30 or more amino acids, e.g. having more than 50 amino acids. In some embodiments, in addition to a primary structure, a protein comprises a secondary, tertiary, or higher structure. The amino acids of the polypeptides are most typically L-amino acids, but may also be D-amino acids, modified amino acids, amino acid analogs, amino acid mimetics, or any combination thereof. Polypeptides may be naturally occurring, synthetically produced, or recombinantly expressed. Polypeptides may be synthetically produced, isolated, recombinantly expressed, or be produced by a combination of methodologies as described above. Polypeptides may also comprise additional groups modifying the amino acid chain, for example, functional groups added via post-translational modification. The polymer may be linear or branched, it may comprise modified amino acids, and it may be interrupted by non-amino acids. The term also encompasses an amino acid polymer that has been modified naturally or by intervention; for example, disulfide bond formation, glycosylation, lipidation, acetylation, phosphorylation, or any other manipulation or modification, such as conjugation with a labeling component.

As used herein, the term “amino acid” refers to an organic compound comprising an amine group, a carboxylic acid group, and a side-chain specific to each amino acid, which serve as a monomeric subunit of a peptide. An amino acid includes the 20 standard, naturally occurring or canonical amino acids as well as non-standard amino acids. The standard, naturally-occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). An amino acid may be an L-amino acid or a D-amino acid. Non-standard amino acids may be modified amino acids, amino acid analogs, amino acid mimetics, non-standard proteinogenic amino acids, or non-proteinogenic amino acids that occur naturally or are chemically synthesized. Examples of non-standard amino acids include, but are not limited to, selenocysteine, pyrrolysine, and N-formylmethionine, β-amino acids, Homo-amino acids, Proline and Pyruvic acid derivatives, 3-substituted alanine derivatives, glycine derivatives, ring-substituted phenylalanine and tyrosine derivatives, linear core amino acids, N-methyl amino acids.

As used herein, the term “post-translational modification” refers to modifications that occur on a peptide after its translation, e.g., translation by ribosomes, is complete. A post-translational modification may be a covalent chemical modification or enzymatic modification. Examples of post-translation modifications include, but are not limited to, acylation, acetylation, alkylation (including methylation), biotinylation, butyrylation, carbamylation, carbonylation, deamidation, deiminiation, diphthamide formation, disulfide bridge formation, eliminylation, flavin attachment, formylation, gamma-carboxylation, glutamylation, glycylation, glycosylation, glypiation, heme C attachment, hydroxylation, hypusine formation, iodination, isoprenylation, lipidation, lipoylation, malonylation, methylation, myristolylation, oxidation, palmitoylation, pegylation, phosphopantetheinylation, phosphorylation, prenylation, propionylation, retinylidene Schiff base formation, S-glutathionylation, S-nitrosylation, S-sulfenylation, selenation, succinylation, sulfination, ubiquitination, and C-terminal amidation. A post-translational modification includes modifications of the amino terminus and/or the carboxyl terminus of a peptide. Modifications of the terminal amino group include, but are not limited to, des-amino, N-lower alkyl, N-di-lower alkyl, and N-acyl modifications. Modifications of the terminal carboxy group include, but are not limited to, amide, lower alkyl amide, dialkyl amide, and lower alkyl ester modifications (e.g., wherein lower alkyl is C₁-C₄ alkyl). A post-translational modification also includes modifications, such as but not limited to those described above, of amino acids falling between the amino and carboxy termini. The term post-translational modification can also include peptide modifications that include one or more detectable labels.

As used herein, the term “binding agent” refers to a nucleic acid molecule, a peptide, a polypeptide, a protein, carbohydrate, or a small molecule that binds to, associates, unites with, recognizes, or combines with a binding target, e.g., a polypeptide or a component or feature of a polypeptide. A binding agent may form a covalent association or non-covalent association with the polypeptide or component or feature of a polypeptide. A binding agent may also be a chimeric binding agent, composed of two or more types of molecules, such as a nucleic acid molecule-peptide chimeric binding agent or a carbohydrate-peptide chimeric binding agent. A binding agent may be a naturally occurring, synthetically produced, or recombinantly expressed molecule. A binding agent may bind to a single monomer or subunit of a polypeptide (e.g., a single amino acid of a polypeptide) or bind to a plurality of linked subunits of a polypeptide (e.g., a di-peptide, tri-peptide, or higher order peptide of a longer peptide, polypeptide, or protein molecule). A binding agent may bind to a linear molecule or a molecule having a three-dimensional structure (also referred to as conformation). For example, an antibody binding agent may bind to linear peptide, polypeptide, or protein, or bind to a conformational peptide, polypeptide, or protein. A binding agent may bind to an N-terminal peptide, a C-terminal peptide, or an intervening peptide of a peptide, polypeptide, or protein molecule. A binding agent may bind to an N-terminal amino acid, C-terminal amino acid, or an intervening amino acid of a peptide molecule. A binding agent may preferably bind to a chemically modified or labeled amino acid (e.g., an amino acid that has been labeled by a chemical reagent) over a non-modified or unlabeled amino acid. For example, a binding agent may preferably bind to an amino acid that has been labeled or modified over an amino acid that is unlabeled or unmodified. A binding agent may bind to a post-translational modification of a peptide molecule. A binding agent may exhibit selective binding to a component or feature of a polypeptide (e.g., a binding agent may selectively bind to one of the 20 possible natural amino acid residues and bind with very low affinity or not at all to the other 19 natural amino acid residues). A binding agent may exhibit less selective binding, where the binding agent is capable of binding or configured to bind to a plurality of components or features of a polypeptide (e.g., a binding agent may bind with similar affinity to two or more different amino acid residues).

As used herein, the term “detectable label” refers to a substance which can indicate the presence of another substance when associated with it. The detectable label can be a substance that is linked to or incorporated into the substance to be detected. In some embodiments, a detectable label is suitable for allowing for detection and also quantification, for example, a detectable label that emitting a detectable and measurable signal. Detectable labels include any labels that can be utilized and are compatible with the provided polypeptide analysis assay format and include, but not limited to, a bioluminescent label, a biotin/avidin label, a chemiluminescent label, a chromophore, a coenzyme, a dye, an electro-active group, an electrochemiluminescent label, an enzymatic label (e.g. alkaline phosphatase, luciferase or horseradish peroxidase), a fluorescent label, a latex particle, a magnetic particle, a metal, a metal chelate, a phosphorescent dye, a protein label, a radioactive element or moiety, and a stable radical.

As used herein, the term “linker” refers to one or more of a nucleotide, a nucleotide analog, an amino acid, a peptide, a polypeptide, a polymer, or a non-nucleotide chemical moiety that is used to join two molecules. A linker may be used to join a first detection agent with a polypeptide, a binding agent with a second detection agent, a polypeptide with a support, a detection agent with a support, etc. A linker may be used to join a DNA tag (e.g. a recording tag) with a polypeptide or a DNA tag with a support. In certain embodiments, a linker joins two molecules via enzymatic reaction or chemistry reaction (e.g., click chemistry).

The term “ligand” as used herein refers to any molecule or moiety connected to the compounds described herein. “Ligand” may refer to one or more ligands attached to a compound. In some embodiments, the ligand is a pendant group or binding site (e.g., the site to which the binding agent binds).

As used herein, the term “proteome” can include the entire set of proteins, polypeptides, or peptides (including conjugates or complexes thereof) expressed by a genome, cell, tissue, or organism at a certain time, of any organism. In one aspect, it is the set of expressed proteins in a given type of cell or organism, at a given time, under defined conditions. Proteomics is the study of the proteome. For example, a “cellular proteome” may include the collection of proteins found in a particular cell type under a particular set of environmental conditions, such as exposure to hormone stimulation. An organism's complete proteome may include the complete set of proteins from all of the various cellular proteomes. A proteome may also include the collection of proteins in certain sub-cellular biological systems. For example, all of the proteins in a virus can be called a viral proteome. As used herein, the term “proteome” include subsets of a proteome, including but not limited to a kinome; a secretome; a receptome (e.g., GPCRome); an immunoproteome; a nutriproteome; a proteome subset defined by a post-translational modification (e.g., phosphorylation, ubiquitination, methylation, acetylation, glycosylation, oxidation, lipidation, and/or nitrosylation), such as a phosphoproteome (e.g., phosphotyrosine-proteome, tyrosine-kinome, and tyrosine-phosphatome), a glycoproteome, etc.; a proteome subset associated with a tissue or organ, a developmental stage, or a physiological or pathological condition; a proteome subset associated a cellular process, such as cell cycle, differentiation (or de-differentiation), cell death, senescence, cell migration, transformation, or metastasis; or any combination thereof. As used herein, the term “proteomics” refers to quantitative analysis of the proteome within cells, tissues, and bodily fluids, and the corresponding spatial distribution of the proteome within the cell and within tissues. Additionally, proteomics studies include the dynamic state of the proteome, continually changing in time as a function of biology and defined biological or chemical stimuli.

The terminal amino acid at one end of a peptide or polypeptide chain that has a free amino group is referred to herein as the “N-terminal amino acid” (NTAA). The terminal amino acid at the other end of the chain that has a free carboxyl group is referred to herein as the “C-terminal amino acid” (CTAA). The amino acids making up a peptide may be numbered in order, with the peptide being “n” amino acids in length. As used herein, NTAA is considered the n^{th} amino acid (also referred to herein as the “n NTAA”). Using this nomenclature, the next amino acid is the n−1 amino acid, then the n−2 amino acid, and so on down the length of the peptide from the N-terminal end to C-terminal end. In certain embodiments, an NTAA, CTAA, or both may be modified or labeled with a moiety or a chemical moiety.

As used herein, the term “barcode” refers to a molecule providing a unique identifier tag or origin information for a polypeptide, a binding agent, a set of binding agents from a binding cycle, a sample polypeptides, a set of samples, polypeptides within a compartment (e.g., droplet, bead, or separated location), polypeptides within a set of compartments, a fraction of polypeptides, a set of polypeptide fractions, a spatial region or set of spatial regions, a library of polypeptides, or a library of binding agents. A “nucleic acid barcode” refers to a nucleic acid molecule of about 2 to about 30 bases (e.g., 2, 3, 4, 5, 6, 7, 8, 9, 10, 11, 12, 13, 14, 15, 16, 17, 18, 19, 20, 21, 22, 23, 24, 25, 26, 27, 28, 29 or 30 bases). A “peptide barcode” or “amino acid barcode” refers to a sequence of amino acids that can have a length of at least, for example, 1, 2, 3, 4, 5, 6, 7, 8, 9, 10, 11, 12, 13, 14, 15, 16, 17, 18, 19, 20, 21, 22, 23, 24, 25, 30, 40, 50, 75, or 100 amino acids. A specific peptide barcode can be distinguished from other peptide barcodes by having a different length, sequence, or other physical property (for example, hydrophobicity). A barcode can be an artificial sequence or a naturally occurring sequence. In certain embodiments, each barcode within a population of barcodes is different. In other embodiments, a portion of barcodes in a population of barcodes is different, e.g., at least about 10%, 15%, 20%, 25%, 30%, 35%, 40%, 45%, 50%, 55%, 60%, 65%, 70%, 75%, 80%, 85%, 90%, 95%, 97%, or 99% of the barcodes in a population of barcodes is different. A population of barcodes may be randomly generated or non-randomly generated. In certain embodiments, a population of barcodes are error-correcting or error-tolerant barcodes. Barcodes can be used to computationally deconvolute the multiplexed sequencing data and identify sequence reads derived from an individual polypeptide, sample, library, etc.

As used herein, the term “primer extension”, also referred to as “polymerase extension”, refers to a reaction catalyzed by a nucleic acid polymerase (e.g., DNA polymerase) whereby a nucleic acid molecule (e.g., oligonucleotide primer, spacer sequence) that anneals to a complementary strand is extended by the polymerase, using the complementary strand as template.

As used herein, the term “unique molecular identifier” or “UMI” refers to a nucleic acid molecule of about 3 to about 40 bases (3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bases) in length providing a unique identifier tag for each macromolecule, polypeptide or binding agent to which the UMI is linked. A polypeptide UMI can be used to accurately count originating polypeptide molecules by collapsing NGS reads to unique UMIs. A binding agent UMI can be used to identify each individual molecular binding agent that binds to a particular polypeptide. For example, a UMI can be used to identify the number of individual binding events for a binding agent specific for a single amino acid that occurs for a particular peptide molecule. It is understood that when UMI and barcode are both referenced in the context of a binding agent or polypeptide, that the barcode refers to identifying information other that the UMI for the individual binding agent or polypeptide (e.g., sample barcode, compartment barcode, binding cycle barcode).

As used herein, the term “universal priming site” or “universal primer” or “universal priming sequence” refers to a nucleic acid molecule, which may be used for library amplification and/or for sequencing reactions. A universal priming site may include, but is not limited to, a priming site (primer sequence) for PCR amplification, flow cell adaptor sequences that anneal to complementary oligonucleotides on flow cell surfaces enabling bridge amplification in some next generation sequencing platforms, a sequencing priming site, or a combination thereof. Universal priming sites can be used for other types of amplification, including those commonly used in conjunction with next generation digital sequencing. The term “forward” when used in context with a “universal priming site” or “universal primer” may also be referred to as “5′” or “sense”. The term “reverse” when used in context with a “universal priming site” or “universal primer” may also be referred to as “3′” or “antisense”.

As used herein, the term “solid support”, “solid surface”, or “solid substrate”, or “sequencing substrate”, or “substrate” refers to any solid material, including porous and non-porous materials, to which a polypeptide can be associated directly or indirectly, by any means known in the art, including covalent and non-covalent interactions, or any combination thereof. A solid support may be two-dimensional (e.g., planar surface) or three-dimensional (e.g., gel matrix or bead). A solid support can be any support surface including, but not limited to, a bead, a microbead, an array, a glass surface, a silicon surface, a plastic surface, a filter, a membrane, a PTFE membrane, a PTFE membrane, a nitrocellulose membrane, a nitrocellulose-based polymer surface, nylon, a silicon wafer chip, a flow through chip, a flow cell, a biochip including signal transducing electronics, a channel, a microtiter well, an ELISA plate, a spinning interferometry disc, a nitrocellulose membrane, a nitrocellulose-based polymer surface, a polymer matrix, a nanoparticle, or a microsphere. Materials for a solid support include but are not limited to acrylamide, agarose, cellulose, dextran, nitrocellulose, glass, gold, quartz, polystyrene, polyethylene vinyl acetate, polypropylene, polyester, polymethacrylate, polyacrylate, polyethylene, polyethylene oxide, polysilicates, polycarbonates, poly vinyl alcohol (PVA), Teflon, fluorocarbons, nylon, silicon rubber, polyanhydrides, polyglycolic acid, polyvinylchloride, polylactic acid, polyorthoesters, functionalized silane, polypropylfumerate, collagen, glycosaminoglycans, polyamino acids, dextran, or any combination thereof. Solid supports further include thin film, membrane, bottles, dishes, fibers, woven fibers, shaped polymers such as tubes, particles, beads, microspheres, microparticles, or any combination thereof. For example, when solid surface is a bead, the bead can include, but is not limited to, a ceramic bead, a polystyrene bead, a polymer bead, a polyacrylate bead, a methylstyrene bead, an agarose bead, a cellulose bead, a dextran bead, an acrylamide bead, a solid core bead, a porous bead, a paramagnetic bead, a glass bead, a controlled pore bead, a silica-based bead, or any combinations thereof. A bead may be spherical or an irregularly shaped. A bead or support may be porous. A bead's size may range from nanometers, e.g., 100 nm, to millimeters, e.g., 1 mm. In certain embodiments, beads range in size from about 0.2 micron to about 200 microns, or from about 0.5 micron to about 5 micron. In some embodiments, beads can be about 1, 1.5, 2, 2.5, 2.8, 3, 3.5, 4, 4.5, 5, 5.5, 6, 6.5, 7, 7.5, 8, 8.5, 9, 9.5, 10, 10.5, 15, or 20 μm in diameter. In certain embodiments, “a bead” solid support may refer to an individual bead or a plurality of beads. In some embodiments, the solid surface is a nanoparticle. In certain embodiments, the nanoparticles range in size from about 1 nm to about 500 nm in diameter, for example, between about 1 nm and about 20 nm, between about 1 nm and about 50 nm, between about 1 nm and about 100 nm, between about 10 nm and about 50 nm, between about 10 nm and about 100 nm, between about 10 nm and about 200 nm, between about 50 nm and about 100 nm, between about 50 nm and about 150, between about 50 nm and about 200 nm, between about 100 nm and about 200 nm, or between about 200 nm and about 500 nm in diameter. In some embodiments, the nanoparticles can be about 10 nm, about 50 nm, about 100 nm, about 150 nm, about 200 nm, about 300 nm, or about 500 nm in diameter. In some embodiments, the nanoparticles are less than about 200 nm in diameter.

As used herein, the term “nucleic acid molecule” or “polynucleotide” refers to a single- or double-stranded polynucleotide containing deoxyribonucleotides or ribonucleotides that are linked by 3′-5′ phosphodiester bonds, as well as polynucleotide analogs. A nucleic acid molecule includes, but is not limited to, DNA, RNA, and cDNA. A polynucleotide analog may possess a backbone other than a standard phosphodiester linkage found in natural polynucleotides and, optionally, a modified sugar moiety or moieties other than ribose or deoxyribose. Polynucleotide analogs contain bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide. Examples of polynucleotide analogs include, but are not limited to xeno nucleic acid (XNA), bridged nucleic acid (BNA), glycol nucleic acid (GNA), peptide nucleic acids (PNAs), γPNAs, morpholino polynucleotides, locked nucleic acids (LNAs), threose nucleic acid (TNA), 2′-O-Methyl polynucleotides, 2′-O-alkyl ribosyl substituted polynucleotides, phosphorothioate polynucleotides, and boronophosphate polynucleotides. A polynucleotide analog may possess purine or pyrimidine analogs, including for example, 7-deaza purine analogs, 8-halopurine analogs, 5-halopyrimidine analogs, or universal base analogs that can pair with any base, including hypoxanthine, nitroazoles, isocarbostyril analogues, azole carboxamides, and aromatic triazole analogues, or base analogs with additional functionality, such as a biotin moiety for affinity binding. In some embodiments, the nucleic acid molecule or oligonucleotide is a modified oligonucleotide. In some embodiments, the nucleic acid molecule or oligonucleotide is a DNA with pseudo-complementary bases, a DNA with protected bases, an RNA molecule, a BNA molecule, an XNA molecule, a LNA molecule, a PNA molecule, a γPNA molecule, or a morpholino DNA, or a combination thereof. In some embodiments, the nucleic acid molecule or oligonucleotide is backbone modified, sugar modified, or nucleobase modified. In some embodiments, the nucleic acid molecule or oligonucleotide has nucleobase protecting groups such as Alloc, electrophilic protecting groups such as thiranes, acetyl protecting groups, nitrobenzyl protecting groups, sulfonate protecting groups, or traditional base-labile protecting groups.

As used herein, “nucleic acid sequencing” means the determination of the order of nucleotides in a nucleic acid molecule or a sample of nucleic acid molecules. Similarly, “polypeptide sequencing” means the determination of the identity and order of at least a portion of amino acids in the polypeptide molecule or in a sample of polypeptide molecules.

As used herein, “next generation sequencing” refers to high-throughput sequencing methods that allow the sequencing of millions to billions of molecules in parallel. Examples of next generation sequencing methods include sequencing by synthesis, sequencing by ligation, sequencing by hybridization, polony sequencing, ion semiconductor sequencing, and pyrosequencing. By attaching primers to a solid substrate and a complementary sequence to a nucleic acid molecule, a nucleic acid molecule can be hybridized to the solid substrate via the primer and then multiple copies can be generated in a discrete area on the solid substrate by using polymerase to amplify (these groupings are sometimes referred to as polymerase colonies or polonies). Consequently, during the sequencing process, a nucleotide at a particular position can be sequenced multiple times (e.g., hundreds or thousands of times)—this depth of coverage is referred to as “deep sequencing.” Examples of high throughput nucleic acid sequencing technology include platforms provided by Illumina, BGI, Qiagen, Thermo-Fisher, and Roche, including formats such as parallel bead arrays, sequencing by synthesis, sequencing by ligation, capillary electrophoresis, electronic microchips, “biochips,” microarrays, parallel microchips, and single-molecule arrays (See e.g., Service, Science (2006) 311:1544-1546).

As used herein, “single molecule sequencing” or “third generation sequencing” refers to next-generation sequencing methods wherein reads from single molecule sequencing instruments are generated by sequencing of a single molecule of DNA. Unlike next generation sequencing methods that rely on amplification to clone many DNA molecules in parallel for sequencing in a phased approach, single molecule sequencing interrogates single molecules of DNA and does not require amplification or synchronization. Single molecule sequencing includes methods that need to pause the sequencing reaction after each base incorporation (‘wash-and-scan’ cycle) and methods which do not need to halt between read steps. Examples of single molecule sequencing methods include single molecule real-time sequencing (Pacific Biosciences), nanopore-based sequencing (Oxford Nanopore), duplex interrupted nanopore sequencing, and direct imaging of DNA using advanced microscopy.

As used herein, “analyzing” the polypeptide means to identify, detect, quantify, characterize, distinguish, or a combination thereof, all or a portion of the components of the polypeptide. For example, analyzing a peptide, polypeptide, or protein includes determining all or a portion of the amino acid sequence (contiguous or non-continuous) of the peptide. Analyzing a polypeptide also includes partial identification of a component of the polypeptide. For example, partial identification of amino acids in the polypeptide protein sequence can identify an amino acid in the protein as belonging to a subset of possible amino acids. Analysis typically begins with analysis of the n NTAA, and then proceeds to the next amino acid of the peptide (i.e., n−1, n−2, n−3, and so forth). This is accomplished by elimination of the n NTAA, thereby converting the n−1 amino acid of the peptide to an N-terminal amino acid (referred to herein as the “n−1 NTAA”). Analyzing the peptide may also include determining the presence and frequency of post-translational modifications on the peptide, which may or may not include information regarding the sequential order of the post-translational modifications on the peptide. Analyzing the peptide may also include determining the presence and frequency of epitopes in the peptide, which may or may not include information regarding the sequential order or location of the epitopes within the peptide. Analyzing the peptide may include combining different types of analysis, for example obtaining epitope information, amino acid sequence information, post-translational modification information, or any combination thereof.

The term “joining” or “attaching” one substance to another substance means connecting or linking these substances together utilizing one or more covalent bond(s) and/or non-covalent interactions. Some examples of non-covalent interactions include hydrogen bonding, hydrophobic binding, and Van der Waals forces. Joining can be direct or indirect, such as via a linker. In preferred embodiments, joining two or more substances together would not impair structure or functional activities of the joined substances. The term “associated with” (e.g. one substance is associated with to another substance) means bringing two substances together, so they can participate in the methods described herein. In preferred embodiments, association of two substances preserves their structures and functional activities. Association can be direct or indirect. When one substance is directly associated with another substance, it is equivalent to one substance being joined or attached to another substance. Indirect association means that two substances are brought together by means other than direct joining or attachment. For example, in some embodiments, the polypeptide may be associated with the first detection agent via a solid support (both the polypeptide and the first detection agent are independently attached to the solid support). In some embodiments, indirect association implies that two substances are co-localized with each other, or located in a close proximity with each other.

The term “sequence identity” is a measure of identity between polypeptides at the amino acid level, and a measure of identity between nucleic acids at nucleotide level. The polypeptide sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned. “Sequence identity” means the percentage of identical subunits at corresponding positions in two sequences when the two sequences are aligned to maximize subunit matching, i.e., taking into account gaps and insertions. For example, the BLAST algorithm (NCBI) calculates percent sequence identity and performs a statistical analysis of the similarity and identity between the two sequences. The software for performing BLAST analysis is publicly available through the National Center for Biotechnology Information (NCBI) website.

The term “unmodified” (also “wild-type” or “native”) as used herein is used in connection with biological materials such as nucleic acid molecules and proteins (e.g., cleavase), refers to those which are found in nature and not modified by human intervention.

As used herein, a polynucleotide or polypeptide variant, mutant, homologue, or modified version include polynucleotides or polypeptides that share nucleic acid or amino acid sequence identity with a reference polynucleotide or polypeptide. For example, variant or modified polypeptide generally exhibits about 25%, 30%, 40%, 50%, 60%, 70%, 80%, 85%, 86%, 87%, 88%, 89%, 90%, 91%, 92%, 93%, 94%, 95%, 96%, 97%, 98%, 99% or more sequence identity to a corresponding wild-type or unmodified polypeptide. The term “modified” or “engineered” (or “variant” or mutant”) as used in reference to polynucleotides and polypeptides implies that such molecules are created by human intervention and/or they are non-naturally occurring. A variant, mutant or modified polypeptide is not limited to any variant, mutant or modified polypeptide made or generated by a particular method of making and includes, for example, a variant, mutant or modified polypeptide made or generated by genetic selection, protein engineering, directed evolution, de novo recombinant DNA techniques, or combinations thereof. A mutant, variant or modified polypeptide is altered in primary amino acid sequence by substitution, addition, or deletion of amino acid residues. In some embodiments, variants of a polypeptide displaying only non-substantial or negligible differences in structure can be generated by making conservative amino acid substitutions in the modified polypeptide. By doing this, modified polypeptide variants that comprise a sequence having at least 90% (90%, 91%, 92%, 93%, 94%, 95%, 96%, 97%, 98%, and 99%) sequence identity with the modified polypeptide sequences can be generated, retaining at least one functional activity of the polypeptide. Examples of conservative amino acid changes are known in the art. Examples of non-conservative amino acid changes that are likely to cause major changes in protein structure are those, for example, that cause substitution of a hydrophilic residue to a hydrophobic residue. Methods of making targeted amino acid substitutions, deletions, truncations, and insertions are generally known in the art. For example, amino acid sequence variants can be prepared by mutations in the DNA. Methods for polynucleotide alterations are well known in the art, for example, Kunkel et al. (1987) Methods in Enzymol. 154:367-382; U.S. Pat. No. 4,873,192 and the references cited therein.

It is understood that aspects and embodiments of the invention described herein include “consisting of” and/or “consisting essentially of” aspects and embodiments.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Other objects, advantages and features of the present invention will become apparent from the following specification taken in conjunction with the accompanying drawings.

### I. METHOD FOR ANALYZING POLYPEPTIDES

Provided herein are methods for analyzing a polypeptide, including providing a polypeptide and an associated first detection agent attached to a solid support; contacting the polypeptide with a binding agent capable of binding to the polypeptide, wherein the binding agent is associated with a second detection agent, whereby binding between the polypeptide and the binding agent brings the first detection agent and the second detection agent into sufficient proximity to interact with each other and generate a detectable label; and detecting a signal generated by the detectable label. In some embodiments, the contacting of the polypeptide with a binding agent capable of binding to the polypeptide and detecting the signal is repeated sequentially one or more times. In some aspects, a plurality of binding agents is contacted with a single polypeptide or a plurality of polypeptides for analysis. The plurality of binding agents may include a mixture of binding agents, at least some of which are associated with a second detection agent. In preferred embodiments, the methods described herein are performed on polypeptide(s) immobilized on a surface, e.g. any suitable material, including porous and non-porous materials, a planar surface, etc.

In some cases, the provided methods are advantageous over other detection methods for immobilized molecules, such as other single molecule analysis methods. In some examples, some exemplary advantages of the provided methods include reduced non-specific background signals and/or allowing signal amplification. In some embodiments, the use of multi-component signal generation system and methods (e.g., two-component detection agents or split detection agents) allows for some such advantages. In some instances, the provided methods allow control over generation of the detectable signal. For example, a detectable signal is not generated until a particular component is added to the sample being analyzed. In some embodiments, the methods described herein can be applied to identifying one or more binding events between a plurality of binding agents and a plurality of polypeptides immobilized on a solid support. Identifying one or more binding events by methods described herein provides a higher signal-to-noise ratio than generated by other methods known in the art, since utilizing the described two-component detection agents offers a reduced non-specific background signal, since binding agents unspecifically bound to the solid support are unable to generate a detectable signal.

In some embodiments, solid support used for immobilization of a polypeptide in the claimed methods does not comprise polypeptide(s). In some embodiments, solid support used for immobilization of a polypeptide in the claimed methods does not comprise polynucleotide(s).

In one embodiment, a method is disclosed for analyzing a polypeptide, comprising providing a polypeptide and an associated first detection agent joined to a solid support, the polypeptide having an N-terminal amino acid (NTAA). The polypeptide is contacted with a binding agent capable of binding to the NTAA, wherein the binding agent comprises a second detection agent, whereby binding between the polypeptide and the binding agent brings the first detection agent and the second detection agent into sufficient proximity to generate a detectable label, which is capable of generating a signal. The signal generated by the detectable label is then detected or observed. In some aspects, the binding between the polypeptide and the binding agent is reversible. For example, the binding agent may be released or removed from the polypeptide. In some embodiments, the NTAA is removed from the polypeptide after the signal is generated and detected, thereby yielding a newly exposed NTAA, and the above steps are repeated on the newly exposed NTAA.

Provided herein are methods which includes a polypeptide associated with a first detection agent and a binding agent associated with a second detection agent. Any first and second detection agents (e.g., proteins, nucleic acids, carbohydrates, small molecules) that can provide, form, become, or generate a detectable label, when brought into sufficient proximity of each other or co-localized may be used in the practice of the disclosed method. In some embodiments, the first and/or second detection agent is or comprises a nucleic acid, peptide, antibody, aptamer or small-molecule compound. Non-limiting examples of detection agents (e.g., first or second detection agents) which can be utilized in this manner include: multi-component detection agents; split proteins (such as split enzymes); affinity pairs; fluorophore or chromophore pairs, allosterically modified proteins, proteins comprising blocking groups, or repressor/inducer protein pairs, two molecules which when brought into sufficient proximity can be detected by a third molecule, or any combinations thereof. In some embodiments, the multi-component detection system includes the multi-component detection agents and any activating agents or blocking molecules.

In some embodiments, the first detection agent and the second detection agent, when brought into sufficient proximity, forms a detectable label. In some aspects, the first detection agent and the second detection agent, when brought into sufficient proximity, forms a detectable label precursor, which requires activating the detectable label precursor to form a detectable label. In other embodiments, the detectable label is generated when inhibition of the first and/or second detection agent is removed. For example, the detectable label can be generated when the second detection agent displaces a repressor protein or a blocking molecule from the first detection agent or cleaves a repressor protein or a blocking molecule bound to the first detection agent. In another example, the detectable label can be generated when the first detection agent displaces a repressor protein or a blocking molecule from the second detection agent or cleaves a repressor protein or a blocking molecule bound to the second detection agent.

In some embodiments, the detectable label includes a first detection agent that is configured to generate a detectable signal. In some embodiments, the detectable label includes a second detection agent that is configured to generate a detectable signal. In some embodiments, the detectable label includes a first detection agent joined or associated with a second detection agent that is configured to generate a detectable signal. In some further embodiments, the detectable label generated by the first and/or second detectable label is not active or does not generate a signal until an activating agent is provided or inhibition is removed. In some cases, binding between the polypeptide and the binding agent brings the first detection agent and the second detection agent into sufficient proximity such that the first and/or second detection agents become, form, or generate a detectable label.

In some embodiments, the detectable label is selected from a bioluminescent label, a biotin/avidin label, a chemiluminescent label, a chromophore, a coenzyme, a dye, an electro active group, an electrochemiluminescent label, an enzymatic label, a fluorescent label, a latex particle, a magnetic particle, a metal, a metal chelate, a phosphorescent dye, a protein label, a radioactive element or moiety, and a stable radical. In some cases, the detectable label is selected from a bioluminescent label, a chemiluminescent label, a chromophore label, an enzymatic label, and a fluorescent label.

In some embodiments, the method further includes providing the plurality of polypeptides with a first detection agent. For example, if a sample is obtained, the sample is treated and processed to provide the polypeptides with a first detection agent. An attachment step may be performed to join the first detection agent to the polypeptides. In some cases, each polypeptide or a majority of polypeptides are provided and associated with a first detection agent. In some aspects, the plurality of polypeptides is provided with a first detection agent during or prior to providing the polypeptide and the associated first detection agent joined to a support. In some particular embodiments, the polypeptides are immobilized to the support after providing the polypeptides with the first detection agent.

As described herein, the first detection agent can be any molecule (e.g., protein, nucleic acid, carbohydrate, small molecule, etc.) capable of direct or indirect detection. In some embodiments, the first detection agent is a protein. In some embodiments, the first detection agent is an enzyme, antibody, aptamer, affinity molecule, fluorophore, chromophore or molecule comprising a repressor protein or blocking molecule. As described herein, the second detection agent can be any molecule (e.g., protein, nucleic acid, carbohydrate, small molecule, etc.) capable of direct or indirect detection. In some embodiments, the second detection agent is a protein. In some embodiments, the second detection agent is an enzyme, antibody, aptamer, affinity molecule, fluorophore, chromophore or molecule comprising a repressor protein or blocking molecule. In some cases, it may be interchangeable which is referred to as the first and second detection agents. For example, the detection agent that is associated with the polypeptide can instead be associated with the binding agent and vice versa.

The “first detection agent” and “second detection agent” are also referred to herein as “multi-component detection agents” or “split detection agents” due to their ability to generate a detectible label configured to generate a signal when in sufficient proximity with each other. Such proximity is associated with the selective binding of the polypeptide (e.g., NTAA) by the cognate binding agent. Conversely, in the absence of such binding (as in the case of contact with a non-cognate binding agent), such detectable label is not formed or generated and a signal is absent, or of a diminished or different nature compared to the signal generated in the case of contact with a cognate binding agent capable of selectively binding to the polypeptide (e.g., NTAA).

In some embodiments, the first and second detection agents are molecules that individually are inactive and/or do not generate a detectable signal. In some examples, when the first and second detection agents are brought into proximity, together they associate and become an active molecule configured to generate a detectable label which generates a signal. In some embodiments, the first detection agent is capable of generating a detectable signal on its own and the second detection agent is an activating molecule that allows the first detection agent to become the detectable label that generates the signal. In some embodiments, the second detection agent is capable of generating a detectable signal on its own and the first detection agent is an activating molecule that allows the second detection agent to become the detectable label that generates the signal. In some cases, the first detection agent is repressed or inhibited by a blocking molecule and the second detection agent removes the repression, allowing the signal to be generated by the detectable label (formed by the first detection agent) (see e.g. FIG. 1E). For example, the second detection agent is configured to cleave the blocking molecule to release the inhibition. In some cases, the second detection agent is repressed by a molecule and the first detection agent removes the repression, allowing the signal to be generated by the detectable label (formed by the second detection agent).

In some embodiments, any proteins or enzymes that loses activity when split, but regains activity when co-localized, may be used in the methods disclosed herein. In some embodiments, the methods of the present invention for determining the amino acid sequence of proteins utilize split proteins. In some embodiments, the first and/or second detection agent may comprise any protein capable of being split into at least two parts and is capable or configured to be reconstituted. For example, proteins capable of being split into at least two parts, and which may be reconstituted when brought into sufficient proximity, may be used in the present disclosure. For example, the first and/or second detection agents may comprise split proteins (e.g., Shekhawat et al., Curr Opin Chem Biol. (2011) 15(6): 789-797; PCT Publication No. WO 2017/189751), split aptamers (e.g., PCT Publication No. WO 2017/044494), or split florescent molecules (e.g., and U.S. Application Publication No. US 2005/0221343; Cabantous et al., Sci Rep. (2013) 3: 2854; Romei et al., Annu Rev Biophys. 2019 May 6; 48: 19-44; Tebo et al., Nat Commun. (2019) 10(1):2822). Such parts may be reconstituted covalently, reversibly covalently or non-covalently. The first and second detection agents can be brought together to become active (e.g., enzymatic activity), thereby becoming a detectable label that generates a signal, such as release of a colorimetric or fluorescent signal. In some aspects, split proteins that have been used in complementation assays, including β-lactamase, β-galactosidase, dihydrofolate reductase, green fluorescent protein, ubiquitin, and TEV protease (e.g., Morrell et al., FEBS (2009) Lett 583, 1684-91) may be used as the detection agents. Representative techniques that may be employed in this regard include Fluorescence Resonance Energy Transfer (FRET) (e.g., when two fluorescent proteins, such as GFP and YFP, come together to generate a FRET signal), as well as Bioluminescence Resonance Energy Transfer (BRET) (e.g., when a luciferase comes together with a YFP to generate a BRET signal). Similarly, a Protein-fragment Complementation Assay (PCA) may be employed, including a Bimolecular fluorescence complementation assay (i.e., when fluorescent proteins are reconstituted, such as disclosed in Hu C D, Kerppola T K. Simultaneous visualization of multiple protein interactions in living cells using multicolor fluorescence complementation analysis. Nat Biotechnol. 2003 May; 21(5):539-45). Non-limiting examples of proteins that can be split and used herein, and/or methods related to the same, include: carbonic anhydrase, T7 RNA polymerase, esterase (Jones K A, et al., Development of a Split Esterase for Protein-Protein Interaction-Dependent Small-Molecule Activation. ACS Cent Sci. 2019 Nov. 27; 5(11):1768-1776), SNAP-tag (Mie et al., Analyst, 137:4760-4765, 2012), dihydrofolate reductase (DHFR; Pelletier J N, et al., Oligomerization domain-directed reassembly of active dihydrofolate reductase from rationally designed fragments. Proc Natl Acad Sci USA. 1998 Oct. 13; 95(21):12141-6), beta-lactamase (Galarneau A, et al., Beta-lactamase protein fragment complementation assays as in vivo and in vitro sensors of protein protein interactions. Nat Biotechnol. 2002 June; 20(6):619-22), yeast Gal4 (as in the classical yeast two-hybrid system), split TEV (Tobacco etch virus protease; Wehr M C, et al., Monitoring regulated protein-protein interactions using split TEV. Nat Methods. 2006 December; 3(12):985-93), luciferase, including ReBiL (recombinase enhanced bimolecular luciferase), ubiquitin, GFP (split-GFP), EGFP (enhanced green fluorescent protein), LacZ (beta-galactosidase), infrared fluorescent protein IFP1.4, an engineered chromophore-binding domain (CBD) of a bacteriophytochrome from Deinococcus radiodurans, and Focal adhesion kinase (FAK). Recently, a split recombinase coupled with photodimers, where blue light brings the split protein together to form a functional recombinase was described, demonstrating a light-directed split enzyme recapitulation (Sheets M, et al., Light-Inducible Recombinases for Bacterial Optogenetics. ACS Synth Biol, (2020), 9(2): 227-235). Specific split locations for the abovementioned proteins can be extracted from the existing publications or predicted in silico as disclosed in (Dagliyan O, et al., Nat Commun. 2018 Oct. 2; 9(1):4042), and corresponding split fragments can be utilized as first and second detection agents in the claimed methods.

In some embodiments, the first and/or second detection agent is an affinity molecule. In some embodiments, the first and/or second detection agent is a first/second subunit of split affinity molecule. For example, when brought together by binding between the polypeptide and the binding agent, the subunits of the split affinity molecule may be joined or associated to form the detectable label. In some embodiments, the first and/or second detection agent is a fluorophore or chromophore, or a portion thereof. In some examples, the first and/or second detection agent is or comprises a repressor protein or blocking molecule. In some cases, the first and/or second detection agent is an inducer protein. In some embodiments, the first and second detection agents comprise separate portions of a FRET system. In some embodiments, the first and second detection agents comprise separate portions of a BRET system.

In some embodiments, the first and second detection agents are first and second subunits of split fluorescent reporter. In some embodiments, the first and second detection agents comprise separate portions of a bimolecular fluorescence complementation (BiFC) system. The BiFC system is based on the formation of a fluorescent complex by fragments of a fluorescent protein, brought together by the association of two interaction partners fused to the fragments (Kerppola, T. K. Bimolecular fluorescence complementation (BiFC) analysis as a probe of protein interactions in living cells. Annu. Rev. Biophys. 37, 465-487 (2008)). In some embodiments, an immobilized polypeptide and a binding agent are fused to two complementary fragments of a fluorescent protein (FP), which assemble into a functional reporter if the binding agent bind to the immobilized polypeptide. Importantly, the two complementary fragments are not fluorescent when taken separately, so a high contrast can be obtained regardless of the relative proportion of the binding agent and the immobilized polypeptide.

In some examples, the detectable agent (e.g., first or second detection agent) is an enzyme or a first subunit of a split enzyme. In some aspects, the second detection agent is a second subunit of a split enzyme. In some cases, the enzyme or split enzyme can be any enzyme or subunit of any enzyme. In some examples, when brought together by binding between the polypeptide and the binding agent, the enzyme subunits may be joined or associated to form the detectable label. In some embodiments, the detectable label generated is an enzymatic label. The enzyme or split enzyme can be selected from carbonic anhydrase, T7 RNA polymerase, beta-galactosidase, dihydrofolate reductase, beta-lactamase, tobacco etch virus protease, fluorescent protein, fluorescent reporter, luciferase, and horseradish peroxidase. In some embodiments, the enzyme or split enzyme is carbonic anhydrase, T7 RNA polymerase, or beta-galactosidase, fluorescent protein.

In some examples, the first detection agent and the second detection agent comprise polynucleotides that form a split enzyme when brought into proximity. Multiple biosensors have been developed based on split aptamers, split DNAzymes, split rybozymes and split GFP-mimicking light up RNA aptamers, and the components of these sensors can be used as the first detection agent and the second detection agent. For example, GFP-mimicking light up RNA aptamers utilize various GFP-like fluorophores, for example, 3,5-dimethoxy-4-hydroxybenzylidene imidazolinone (DMHBI), 4-dimethylamino-benzylidene imidazolinone (DMABI), 2-hydroxybenzylidene imidazolinone (2-HBI) and 3,5-difluoro-4-hydroxybenzylidene imidazolinone (DFHBI) (Paige, J. et al., (2011) RNA mimics of green fluorescent protein. Science, 333, 642-646). These ligands binds tightly to the nucleic acid aptamers by intercalating or as minor groove binder; they are non-fluorescent in the unbound state, but become fluorescent after incorporation into the aptamer's structure. A split light up RNA aptamer based on DFHBI was published (Rogers, T, et al., Fluorescent monitoring of RNA assembly and processing using the split-spinach aptamer. ACS Synth. Biol., (2015) 4, 162-166). Several examples of fluorescent split aptamer-based biosensors based on thrombin split aptamers and ATP split aptamers were disclosed (Debiais M, et al., Splitting aptamers and nucleic acid enzymes for the development of advanced biosensors. Nucleic Acids Res. 2020 Apr. 17; 48(7):3400-3422). General principle of these biosensors are based on non-covalent binding of a fluorescent molecule to aptamer united after split (Kent, A, et al., General approach for engineering small-molecule-binding DNA split aptamers. Anal. Chem. (2013), 85, 9916-9923). Split enzyme-mimicking DNA aptamers can also be used such as split peroxidase mimicking DNAzymes (Deng M., et al., (2008) Highly effective colorimetric and visual detection of nucleic acids using an asymmetrically split Peroxidase DNAzyme. J. Am. Chem. Soc., 130, 13095-13102).

In some examples, the first detection agent and the second detection agent comprise polypeptides that form a split enzyme when brought into proximity. Multiple examples of functional split enzymes exist in literature, and most of them can be utilized in the claimed methods to generate a detectable label upon interaction of unfunctional split enzyme subunits. In some examples, a first and a second subunits of a split enzyme can assemble into a functional enzyme spontaneously, upon interaction between an immobilized polypeptide associated with the first subunit and a binding agent joined to the second subunit. In some examples, assembly of the first and second subunits of a split enzyme is driven by an activating agent or light (Spencer, D. M., et al., Controlling signal-transduction with synthetic ligands. Science 262, 1019-1024 (1993); Levskaya, A., et al., Spatiotemporal control of cell signaling using a light-switchable protein interaction. Nature 461, 997-1001 (2009); Kennedy, M. et al., Rapid blue-light-mediated induction of protein interactions in living cells. Nat. Methods 7, 973-U948 (2010)).

Further, appropriate split sites in enzymes can be successfully predicted computationally based on the number of factors, determined by the analysis of previously published examples of functional split proteins. Successful split sites avoided the major split energy minima and located in surface-exposed, evolutionarily non-conserved loops (Dagliyan 0, et al., Computational design of chemogenetic and optogenetic split proteins. Nat Commun. 2018 Oct. 2; 9(1):4042). The split energy profile revealed sites that are critical for protein folding, and therefore should not be used as split sites. Overall, the split energy can serve as an effective tool in finding split sites in enzymes that was demonstrated in several examples, including tyrosine kinase, guanine exchange factor, TEV protease, and guanosine nucleotide dissociation inhibitor (Dagliyan O, et al., Nat Commun. 2018 Oct. 2; 9(1):4042).

In preferred embodiments, a first part of a split enzyme is associated with a polypeptide immobilized on a solid support, and the second part of the split enzyme is connected to a binding agent. In some embodiments, the second part of the split enzyme can be evolved to produce a different signal. In some embodiments, the contacting step comprises contacting the polypeptide with a plurality of binding agents as a mixture; each binding agent is joined to a different second detection agent; and the signal generated by the detectable label is different for each binding agent. For example, green fluorescent protein (GFP) can be split in two parts, and the second part can be evolved by introducing mutations that result in shifts of the fluorescent spectra; when such mutated parts are associated with binding agents, then after binding to polypeptides immobilized on a solid support different signals can be detected (see also Example 3). Similarly, luciferase enzymes from different organisms that emit signal of different wavelengths can be further evolved and split (Paulmurugan R, Gambhir S S. Monitoring protein-protein interactions using split synthetic renilla luciferase protein-fragment-assisted complementation. Anal Chem. 2003 Apr. 1; 75(7):1584-9). For example, Gaussia, Renilla, Cypridina and Red-Firefly luciferases have different emission peaks, and luminescence emissions at different wave lengths can be utilized for different detectable labels.

In some embodiments, generated signal can be different for each binding agent by using an enzyme that is evolved to have different (fast or slow) kinetics of an enzymatic reaction (such as cleavage). A panel of the enzyme variants having mutations that cause change in functional activity (speed of the enzymatic reaction) can be made; the enzymes can be split, so that mutations are located in one split component, and the enzymes are active only after rejoining of the separated components; so the split enzyme components can be used as detection agents.

In some embodiments, the first or second detection agent comprises a cofactor or a coenzyme. In some cases, the cofactor may comprise a non-protein chemical, metal ions, organic compounds, or other chemicals.

In some cases, the first and/or second detection agents require activation to generate a detectable signal. In some embodiments, any proteins or enzymes that loses activity when inhibited by a blocking molecule, but regains activity when released from inhibition, may be used in the methods disclosed herein. In some cases, the first and/or second detection agent comprises a repressor/inducer protein pair, or a portion thereof. In some embodiments, the first and/or second detection agents generate a detectable signal upon introduction to an activating agent or molecule. In some embodiments, any proteins or enzymes that require an activating agent to generate a signal may be used in the methods disclosed herein. In some embodiments, the activating or molecule comprises a chemical reagent, a non-biological reagent, a biological reagent, or a combination thereof. For example, the activating agent comprises a polypeptide or a protein or a metal ion. In some embodiments, the activating agent comprises a cofactor, a non-protein chemical, organic compounds, or other chemicals. In some cases, the first or second detection agent comprises an allosterically modified protein or is configured for conformational change upon binding to an activating molecule or agent. In one example, the second detection agent requires allosteric activation by an activating agent to change conformation to allow interaction with first detection agent (see e.g. FIG. 1F), in order to generate a detectable label. In another example, the first detection agent requires allosteric activation by an activating agent to change conformation to allow interaction with second detection agent.

In some embodiments, the first and/or second detection agents, either individually or together, is configured to require activation by removal or release of inhibition by a blocking or inhibitor molecule. Once activated or removed from inhibition, the first and/or second detection agent may become, form, or generate the detectable label. The blocking or inhibitor molecule may be covalently attached or associated with the first or second detection agents. For example, the removal or release of inhibition can be via removal of the blocking molecule from an active site of the first and/or second detection agent. In some cases, the removal of the blocking molecule can be by any applicable means, such as via displacement of the blocking molecule or cleaving of the blocking molecule. In one example, the second detection agent comprises a cleaving agent (e.g., protein or enzyme) that is configured to cleave a blocking molecule. The removal (via cleavage) of the blocking molecule allows the first detection agent to generate a detectable signal (FIG. 1E). In some aspects, the first or second detection agent configured to cleave the blocking molecule remains inactive until it is brought into sufficient proximity of the blocking molecule. For example, binding of a binding agent to the polypeptide increases the local concentration of the detection agent capable of cleaving the blocking molecule, thus allowing the cleaving to occur. In some specific embodiments, a detection agent capable of cleaving activity requires a further activation step or activation agent to be active. Additional levels of control may be achieved in this manner. In another example, binding of a binding agent to the polypeptide increases the local concentration of the detection agent capable of displacing the blocking molecule, thus allowing the displacing to occur. As shown in FIG. 1D, the second detection agent displaces the blocking molecule when brought into sufficient proximity of the blocking molecule. Various blocking molecules, first detection agents, and second detection agents with suitable binding affinities can be selected and used. In some aspects, any of the provided configurations of the first and second detection agents and any other activating agents or blocking molecules can be switched around and/or can be used in combination.

Both the polypeptide and the first detection agent can be joined to the support, directly or indirectly, by any means known in the art, including covalent and non-covalent interactions, or any combination thereof. For example, the polypeptide and first detection agent may be each joined to a support; alternatively, the polypeptide can be joined to a support, and the first detection agent can be joined to the polypeptide (e.g. the first detection agent can be joined to the support through the polypeptide); alternatively, first detection agent can be joined to a support, and the polypeptide can be joined to the first detection agent (e.g. the polypeptide can be joined to the support through the first detection agent); alternatively, both the first detection agent and the polypeptide can be joined to a support via a linker, wherein the linker is a tri-functional linker that comprises: a moiety for attachment to the polypeptide, a moiety for attachment to the support, and a moiety for attachment to the first detection agent. Alternatively, the polypeptide and first detection agent can be co-localized, directly or indirectly, and joined to a support. For example, the polypeptide and first detection agent can be independently attached to a support in a proximity to each other, so they are associated with each other indirectly, via the support. In this case, the proximity attachment should be configured so that after binding reaction, the first detection agent and the second detection agent can interact with each other and generate a detectable label. In some cases, the first detection agent is directly or indirectly joined to the polypeptide. In some aspects, the second detection agent is directly or indirectly joined to the binding agent. Alternatively, the support can include an agent or coating to facilitate joining, either direct or indirectly, the peptide, the first detection agent, or both, to the support. Any suitable molecule or materials may be employed for this purpose, including proteins, nucleic acids, carbohydrates and small molecules. In some cases, the peptide and/or first detection agent may be joined to the solid support or each other enzymatically or chemically. In some embodiments, the polypeptide and/or first detection agent may be joined to the solid support or each other via ligation. In other embodiments, the peptide and/or first detection agent may be joined to the solid support or each other via affinity binding pairs (e.g., biotin and streptavidin). In some cases, the peptide and/or first detection agent may be joined to the solid support or each other using an unnatural amino acid, such as via a covalent interaction with an unnatural amino acid.

Various configurations can be used for joining the polypeptides and the first detection agents associated or co-localized directly or indirectly with the polypeptide, to the support. In some embodiments, the polypeptide is in proximity of the associated first detection agent. In some particular embodiments, the polypeptide is not directly connected to the first detection agent, but the two are in sufficient proximity of each other. The distance between the polypeptide and associated first detection agent may be adjusted based on the length of the linker and/or the distance between the binding agent and the second detection agent.

Representative exemplary motifs for providing the polypeptide and first detection agent joined to the solid support are illustrated in FIG. 1A. Referring to FIG. 1A, variation A shows polypeptide 112 joined to solid support 110 through linker 114, and first detection agent 120 joined to solid support 110 through linker 122. Referring to variation B, polypeptide 112 joined to solid support 110 through linker 114, and first detection agent 120 is joined, through linker 122 to linker 114. Variation D is similar to variation B, but with first detection agent 120 joined to solid support 110 through linker 122, and polypeptide 112 joined through linker 114 to linker 122. Variation C of FIG. 1A shows polypeptide 112 is joined to solid support 110 through linker 114, with the first detection agent joined to the solid support by binding to polypeptide 112 through linker 122. To this end, it should be understood that these variations are presented for illustrative purposes only, and are not intended to be limiting. For example, while linkers are shown to aid attachment, such linkers are optional and direct attachment between the various components is within the scope of this disclosure. Further, such linkers may join the polypeptide and the first detection agent to the solid support by covalent or non-covalent interactions, or a mixture thereof, and the linker may comprise multiple components.

In some embodiments, a linker is used to join the first detection agent to the support, the polypeptide to the support, the first detection agent to the polypeptide, or some combination thereof. In some embodiments, the linker a moiety to associating with the polypeptide and a moiety for associating with the first detection agent. For example, the joining uses a linker which comprises an azide group, which can react with an alkynyl group in another molecule to facilitate association or binding between the solid support and the other molecule. In some embodiments, the linker comprises a biotin. In some cases, the first detection agent is configured to bind the biotin. In some aspects, the first detection agent is associated with a hapten-binding group. For example, the hapten-binding group is streptavidin. In some examples, the hapten-binding group and the first detection agent are chemically or genetically attached. In some examples, the chemical attachment is a covalent attachment via a linker molecule.

In some embodiments, the linker is a tri-functional linker. For example, the tri-functional linker may include a moiety to associating with the polypeptide; a moiety for associating with the support; and a moiety for associating with the first detection agent. A linker can be any molecule (e.g., protein, nucleic acid, carbohydrate, small molecule, etc.) capable of associating or binding a polypeptide to a solid surface.

In one embodiment, the linker used to join the polypeptide and the first detection agent to the solid support has the following structure (L-1):

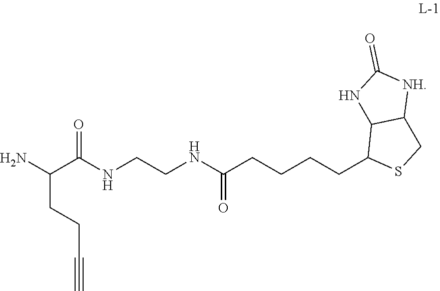 [SMILES: C#CCCC(N)C(=O)NCCNC(=O)CCCCC1SCC2NC(=O)NC21]

Linker L-1 contains an amine group which can bind the polypeptide by, for example, formation of an amide bond with the carboxylate of tryptic peptides using 1-ethyl-3-(3-dimethylaminopropyl) carbodiimide (EDC) coupling. Further, the alkynyl group provides for joining of L-1 a solid support bearing an azide group through click chemistry. Lastly, L-1 also contains biotin, which can be bound by streptavidin linked to the first detection agent. As illustrated in this embodiment, L-1 serves to join both the peptide and the first detection agent to the solid support by both covalent (amide bond formation and click chemistry) and non-covalent binding (biotin-streptavidin interaction), both of which are encompassed within the practice of this disclosure.

The linker can have the following structure:

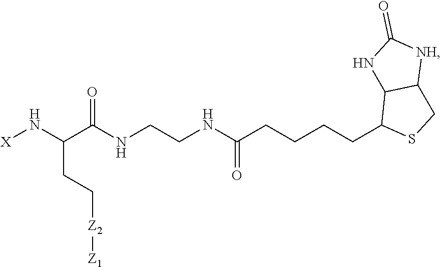

wherein:

X is the polypeptide; and

Z₁-Z₂ is C≡C and is capable of binding to the solid support.

The linker can be trifunctional, as it can (1) associate or bind to a solid support; (2) associate or bind to a polypeptide to be analyzed or sequenced (3) associate or bind to a hapten-binding protein (when the first molecule comprises a hapten molecule). The association or binding can be covalent or non-covalent.

The linker may comprise an amine group, which can form an amide bond with the carboxylate of tryptic peptides via 1-Ethyl-3-(3-dimethylaminopropyl) carbodiimide (EDC) coupling. The alkyne group of the trifunctional reagent allows the association or binding of polypeptides to a solid support bead coated with an azide group through bio-orthogonal click chemistry. In some embodiments, the hapten is a biotin which can be bound by a streptavidin.

In some embodiments, the linker can be prepared by following a solid phase synthesis. For example, Biotin NovaTag resin (Millipore) is deprotected with 20% piperidine to remove the Fmoc group, it is then coupled with N-Fmoc-L-propargylglycine (Sigma) in the presence of HBTU (Sigma). After the Fmoc group is removed by 20% piperidine, the reagent is cleaved from the beads by 95% TFA and purified by HPLC.

In some embodiments, tri-functional linker is an amino acid-based linker, such as lysine-based tri-functional linker. Amino acids provide a unique molecular scaffold to derive “trifunctional” linkers through separate modification of the N-terminus, C-terminus, and sidechain (natural or unnatural). For example, amino acid side chains, may be functionalized with various attachment tags using standard amine modification chemistry or produced with a pre-installed attachment tag (e.g. biotin, desthiobiotin, mTET, photoreactive tags (diazirine, benzophenone, etc.)). C-terminal carboxylates can be converted into reactive esters through standard chemistries (CDI, EDC, etc.), provided the N-terminus is protected to prevent polymerization of the reagent.

The solid support can further include an agent (e.g., reacting agent) or coating to facilitate the direct or indirect binding of a polypeptide, or other reagent of the instant invention, to the solid support. The reacting agent can be any molecule (e.g., protein, nucleic acid, carbohydrate, small molecule). The reacting agent can be an affinity molecule. The reacting agent can be an azide group. In embodiments where the reacting agent is an azide group, the azide group can react with an alkyline group in another molecule to facilitate association or binding between the solid support and the other molecule.

In some aspects, the methods provide herein include forming a complex which can comprise a support, a linker (e.g. first molecule) and a polypeptide. For example, the complex can be formed by reacting the molecule with a solid support to form a linker-solid support complex, and then reacting the linker-solid support complex with the polypeptide. The complex can also be formed by reacting the linker with the polypeptide to form a linker-polypeptide complex, and then reacting the linker-polypeptide complex with the solid support. The association or binding between the linker, support and polypeptide can be covalent or non-covalent. In some embodiments, the complex can be formed by reaction of an amine group in the first molecule and a carboxyl group in the polypeptide. The first complex can be formed via a 1-ethyl-3-(3-dimethylaminopropyl)carbodiimide (EDC) coupling reaction

Provided herein are methods for assaying a polypeptide, protein and/or peptide. The methods of the present invention also permit the detection, quantitation or analysis of a plurality of peptides (two or more peptides) simultaneously, e.g., multiplexing. Simultaneously as used herein refers to detection, quantitation or sequencing of a plurality of peptides in the same assay. The plurality of peptides detected, quantitated and/or analyzed can be present in the same sample, e.g., biological sample, or different samples. The plurality of polypeptides can be derived from the same subject or different subjects. In some embodiments, the method is performed on a plurality of isolated polypeptides from a sample. In some aspects, the polypeptides are of unknown identity. The plurality of polypeptides that are analyzed can be different polypeptides, or the same polypeptide derived from different samples. A plurality of polypeptides includes 2 or more polypeptides, 5 or more polypeptides, 10 or more polypeptides, 50 or more polypeptides, 100 or more polypeptides, 500 or more polypeptides, 1000 or more polypeptides, 5,000 or more polypeptides, 10,000 or more polypeptides, 50,000 or more polypeptides, 100,000 or more polypeptides, 500,000 or more polypeptides, or 1,000,000 or more polypeptides.

Polypeptides for analysis using the provided method may be obtained from a source and treated in various ways. In some cases, the provided methods are useful on macromolecules (e.g., polypeptides) obtained from a sample and are of unknown identity. In some cases, the polypeptides are obtained from a mixture of macromolecules from a sample. A macromolecule can be a large molecule composed of smaller subunits. In certain embodiments, a macromolecule is a protein, a protein complex, polypeptide, peptide, nucleic acid molecule, carbohydrate, lipid, macrocycle, a chimeric macromolecule, or a combination thereof.

In some embodiments, the proteins, polypeptides, or peptides are obtained from a sample that is a biological sample. In some embodiments, the sample comprises but is not limited to, mammalian or human cells, yeast cells, and/or bacterial cells. In some embodiments, the sample contains cells that are from a sample obtained from a multicellular organism. For example, the sample may be isolated from an individual. In some embodiments, the sample may comprise a single cell type or multiple cell types. In some embodiments, the sample may be obtained from a mammalian organism or a human, for example by puncture, or other collecting or sampling procedures. In some embodiments, the sample comprises two or more cells.

In some embodiments, the biological sample may contain whole cells and/or live cells and/or cell debris. In some examples, a suitable source or sample, may include but is not limited to: biological samples, such as biopsy samples, cell cultures, cells (both primary cells and cultured cell lines), sample comprising cell organelles or vesicles, tissues and tissue extracts; of virtually any organism. For example, a suitable source or sample, may include but is not limited to: biopsy; fecal matter; bodily fluids (such as blood, whole blood, serum, plasma, urine, lymph, bile, aqueous humor, breast milk, cerumen (earwax), chyle, chyme, endolymph, perilymph, exudates, cerebrospinal fluid, interstitial fluid, aqueous or vitreous humor, colostrum, sputum, amniotic fluid, saliva, anal and vaginal secretions, gastric acid, gastric juice, lymph, mucus (including nasal drainage and phlegm), pericardial fluid, peritoneal fluid, pleural fluid, pus, rheum, saliva, sebum (skin oil), sputum, synovial fluid, perspiration and semen, a transudate, vomit and mixtures of one or more thereof, an exudate (e.g., fluid obtained from an abscess or any other site of infection or inflammation) or fluid obtained from a joint (normal joint or a joint affected by disease such as rheumatoid arthritis, osteoarthritis, gout or septic arthritis) of virtually any organism, with mammalian-derived samples, including microbiome-containing samples, being preferred and human-derived samples, including microbiome-containing samples, being particularly preferred; environmental samples (such as air, agricultural, water and soil samples); microbial samples including samples derived from microbial biofilms and/or communities, as well as microbial spores; tissue samples including tissue sections, research samples including extracellular fluids, extracellular supernatants from cell cultures, inclusion bodies in bacteria, cellular components including mitochondria and cellular periplasm. In some embodiments, the biological sample comprises a body fluid or is derived from a body fluid, wherein the body fluid is obtained from a mammal or a human. In some embodiments, the sample includes bodily fluids, or cell cultures from bodily fluids.

In some embodiments, the macromolecules (e.g., polypeptides and proteins) may be obtained and prepared from a single cell type or multiple cell types. In some embodiments, the sample comprises a population of cells. In some embodiments, the proteins, polypeptides, or peptides are from a cellular or subcellular component, an extracellular vesicle, an organelle, or an organized subcomponent thereof. The proteins, polypeptides, or peptides may be from organelles, for example, mitochondria, nuclei, or cellular vesicles. In one embodiment, one or more specific types of single cells or subtypes thereof may be isolated. In some embodiments, the sample may include but are not limited to cellular organelles, (e.g., nucleus, golgi apparatus, ribosomes, mitochondria, endoplasmic reticulum, chloroplast, cell membrane, vesicles, etc.).

A peptide may comprise L-amino acids, D-amino acids, or both. A peptide, polypeptide, protein, or protein complex may comprise a standard, naturally occurring amino acid, a modified amino acid (e.g., post-translational modification), an amino acid analog, an amino acid mimetic, or any combination thereof. In some embodiments, a peptide, polypeptide, or protein is naturally occurring, synthetically produced, or recombinantly expressed. In any of the aforementioned peptide embodiments, a peptide, polypeptide, protein, or protein complex may further comprise a post-translational modification. Standard, naturally occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). Non-standard amino acids include selenocysteine, pyrrolysine, and N-formylmethionine, β-amino acids, homo-amino acids, Proline and Pyruvic acid derivatives, 3-substituted Alanine derivatives, Glycine derivatives, ring-substituted Phenylalanine and Tyrosine Derivatives, linear core amino acids, and N-methyl amino acids.

A post-translational modification (PTM) of a peptide, polypeptide, or protein may be a covalent modification or enzymatic modification. Examples of post-translation modifications include, but are not limited to, acylation, acetylation, alkylation (including methylation), biotinylation, butyrylation, carbamylation, carbonylation, deamidation, deiminiation, diphthamide formation, disulfide bridge formation, eliminylation, flavin attachment, formylation, gamma-carboxylation, glutamylation, glycylation, glycosylation (e.g., N-linked, O-linked, C-linked, phosphoglycosylation), glypiation, heme C attachment, hydroxylation, hypusine formation, iodination, isoprenylation, lipidation, lipoylation, malonylation, methylation, myristolylation, oxidation, palmitoylation, pegylation, phosphopantetheinylation, phosphorylation, prenylation, propionylation, retinylidene Schiff base formation, S-glutathionylation, S-nitrosylation, S-sulfenylation, selenation, succinylation, sulfination, ubiquitination, and C-terminal amidation. A post-translational modification includes modifications of the amino terminus and/or the carboxyl terminus of a peptide, polypeptide, or protein. Modifications of the terminal amino group include, but are not limited to, des-amino, N-lower alkyl, N-di-lower alkyl, and N-acyl modifications. Modifications of the terminal carboxy group include, but are not limited to, amide, lower alkyl amide, dialkyl amide, and lower alkyl ester modifications (e.g., wherein lower alkyl is C₁-C₄ alkyl). A post-translational modification also includes modifications, such as but not limited to those described above, of amino acids falling between the amino and carboxy termini of a peptide, polypeptide, or protein. Post-translational modification can regulate a protein's “biology” within a cell, e.g., its activity, structure, stability, or localization. For example, phosphorylation plays an important role in regulation of protein, particularly in cell signaling (Prabakaran et al., 2012, Wiley Interdiscip Rev Syst Biol Med 4: 565-583). In another example, the addition of sugars to proteins, such as glycosylation, has been shown to promote protein folding, improve stability, and modify regulatory function and the attachment of lipids to proteins enables targeting to the cell membrane. A post-translational modification can also include peptide, polypeptide, or protein modifications to include one or more detectable labels.

In certain embodiments, a peptide, polypeptide, or protein can be fragmented. Peptides, polypeptides, or proteins can be fragmented by any means known in the art, including fragmentation by a protease or endopeptidase. In some embodiments, fragmentation of a peptide, polypeptide, or protein is targeted by use of a specific protease or endopeptidase. A specific protease or endopeptidase binds and cleaves at a specific consensus sequence (e.g., TEV protease). In other embodiments, fragmentation of a peptide, polypeptide, or protein is non-targeted or random by use of a non-specific protease or endopeptidase. A non-specific protease may bind and cleave at a specific amino acid residue rather than a consensus sequence (e.g., proteinase K is a non-specific serine protease). In some embodiments, proteinases and endopeptidases, such as those known in the art, can be used to cleave a protein or polypeptide into smaller peptide fragments include proteinase K, trypsin, chymotrypsin, pepsin, thermolysin, thrombin, Factor Xa, furin, endopeptidase, papain, pepsin, subtilisin, elastase, enterokinase, Genenase™ I, Endoproteinase LysC, Endoproteinase AspN, Endoproteinase GluC, etc. (Granvogl et al., 2007, Anal Bioanal Chem 389: 991-1002). In certain embodiments, a peptide, polypeptide, or protein is fragmented by proteinase K, or optionally, a thermolabile version of proteinase K to enable rapid inactivation. In some cases, Proteinase K is stable in denaturing reagents, such as urea and SDS, and enables digestion of completely denatured proteins.

Chemical reagents can also be used to digest proteins into peptide fragments. A chemical reagent may cleave at a specific amino acid residue (e.g., cyanogen bromide hydrolyzes peptide bonds at the C-terminus of methionine residues). Chemical reagents for fragmenting polypeptides or proteins into smaller peptides include cyanogen bromide (CNBr), hydroxylamine, hydrazine, formic acid, BNPS-skatole [2-(2-nitrophenylsulfenyl)-3-methylindole], iodosobenzoic acid, .NTCB+Ni (2-nitro-5-thiocyanobenzoic acid), etc.

In certain embodiments, following enzymatic or chemical cleavage, the resulting peptide fragments are approximately the same desired length, e.g., from about 10 amino acids to about 70 amino acids, from about 10 amino acids to about 60 amino acids, from about 10 amino acids to about 50 amino acids, about 10 to about 40 amino acids, from about 10 to about 30 amino acids, from about 20 amino acids to about 70 amino acids, from about 20 amino acids to about 60 amino acids, from about 20 amino acids to about 50 amino acids, about 20 to about 40 amino acids, from about 20 to about 30 amino acids, from about 30 amino acids to about 70 amino acids, from about 30 amino acids to about 60 amino acids, from about 30 amino acids to about 50 amino acids, or from about 30 amino acids to about 40 amino acids. A cleavage reaction may be monitored, preferably in real time, by spiking the protein or polypeptide sample with a short test FRET (fluorescence resonance energy transfer) peptide comprising a peptide sequence containing a proteinase or endopeptidase cleavage site. In the intact FRET peptide, a fluorescent group and a quencher group are attached to either end of the peptide sequence containing the cleavage site, and fluorescence resonance energy transfer between the quencher and the fluorophore leads to low fluorescence. Upon cleavage of the test peptide by a protease or endopeptidase, the quencher and fluorophore are separated giving a large increase in fluorescence. A cleavage reaction can be stopped when a certain fluorescence intensity is achieved, allowing a reproducible cleavage endpoint to be achieved.

In some aspects, the sample can undergo protein fractionation methods where proteins or peptides are separated by one or more properties such as cellular location, molecular weight, hydrophobicity, isoelectric point, or protein enrichment methods. In some embodiments, a subset of macromolecules (e.g., proteins) within a sample is fractionated such that a subset of the macromolecules is sorted from the rest of the sample. For example, the sample may undergo fractionation methods prior to attachment to a support. Alternatively, or additionally, protein enrichment methods may be used to select for a specific protein or peptide (see, e.g., Whiteaker et al., 2007, Anal. Biochem. 362:44-54, incorporated by reference in its entirety) or to select for a particular post translational modification (see, e.g., Huang et al., 2014. J. Chromatogr. A 1372:1-17, incorporated by reference in its entirety). Alternatively, a particular class or classes of proteins such as immunoglobulins, or immunoglobulin (Ig) isotypes such as IgG, can be affinity enriched or selected for analysis. In the case of immunoglobulin molecules, analysis of the sequence and abundance or frequency of hypervariable sequences involved in affinity binding are of particular interest, particularly as they vary in response to disease progression or correlate with healthy, immune, and/or or disease phenotypes. Overly abundant proteins can also be subtracted from the sample using standard immunoaffinity methods. Depletion of abundant proteins can be useful for plasma samples where over 80% of the protein constituent is albumin and immunoglobulins. Several commercial products are available for depletion of plasma samples of overly abundant proteins, including depletion spin columns that remove top 2-20 plasma proteins (Pierce, Agilent), or PROTIA and PROT20 (Sigma-Aldrich).

In certain embodiments, a protein sample dynamic range can be modulated by fractionating the protein sample using standard fractionation methods, including electrophoresis and liquid chromatography (Zhou et al., 2012, Anal Chem 84(2): 720-734), or partitioning the fractions into compartments (e.g., droplets) loaded with limited capacity protein binding beads/resin (e.g. hydroxylated silica particles) (McCormick, 1989, Anal Biochem 181(1): 66-74) and eluting bound protein. Excess protein in each compartmentalized fraction is washed away. Examples of electrophoretic methods include capillary electrophoresis (CE), capillary isoelectric focusing (CIEF), capillary isotachophoresis (CITP), free flow electrophoresis, gel-eluted liquid fraction entrapment electrophoresis (GELFrEE). Examples of liquid chromatography protein separation methods include reverse phase (RP), ion exchange (IE), size exclusion (SE), hydrophilic interaction, etc. Examples of compartment partitions include emulsions, droplets, microwells, physically separated regions on a flat substrate, etc. Exemplary protein binding beads/resins include silica nanoparticles derivatized with phenol groups or hydroxyl groups (e.g., StrataClean Resin from Agilent Technologies, RapidClean from LabTech, etc.). By limiting the binding capacity of the beads/resin, highly-abundant proteins eluting in a given fraction will only be partially bound to the beads, and excess proteins removed.

A peptide analyzed in accordance with this disclosure may be enriched prior to analysis. Methods for enriching a peptide of interest can include removing the peptide of interest from a sample (direct enrichment) or removing or subtracting other peptides from the sample (indirect enrichment), or both. Enrichment can increase the efficiency of the disclosed methods, improve dynamic range and improve the ability to detect many low abundance proteins in a complex sample. The methods of enrichment can include, but are not limited to, removing abundant species, such as albumin; enrich/subtract specific targeting of particular proteins (e.g. by antibody capture); enrich/subtract by general properties of proteins (e.g. size, pI, hydrophobicity, etc.); enrich/subtract by targeting classes of proteins (e.g. by modification, such as phosphorylated proteins and glycosylated proteins); by ability to bind certain molecules (e.g. DNA binding proteins); ATP binding proteins; enrich/subtract by subcellular localization (e.g. nuclear, mitochondrial, golgi/ER, etc.); enrich/subtract by cellular population (e.g. T-cells, B-cells, etc.) that can be identified & sorted or otherwise captured (e.g. via cell surface markers). Methods and techniques for enrichment include, but are not limited to, centrifugation, chromatography, electrophoresis, binding, filtration, precipitation and degradation.

In some embodiments, a sample of peptides, polypeptides, or proteins can be processed into a physical area or volume e.g., into a compartment. Various processing and/or labeling steps may be performed on the sample prior to performing the binding reaction. In some embodiments, the compartment separates or isolates a subset of macromolecules from a sample of macromolecules. In some examples, the compartment may be an aqueous compartment (e.g., microfluidic droplet), a solid compartment (e.g., picotiter well or microtiter well on a plate, tube, vial, bead), or a separated region on a surface. In some cases, a compartment may comprise one or more beads to which macromolecules may be immobilized. In some embodiments, macromolecules in a compartment is labeled with a barcode. For example, the macromolecules in one compartment can be labeled with the same barcode or macromolecules in multiple compartments can be labeled with the same barcode. See e.g., Valihrach et al., Int J Mol Sci. 2018 Mar. 11; 19(3). pii: E807.

The polypeptides and the first detection agent can be joined to a support, directly or indirectly, by any means known in the art. For example, the peptide and/or first detection agent may be joined to the support, joined to each other, or the polypeptide and first detection agent can be co-localized, directly or indirectly, and joined to a support. In some cases, it is desirable to use a support with a large carrying capacity to immobilize a large number of polypeptides. In some embodiments, it is preferred to immobilize the polypeptides using a three-dimensional support (e.g., a porous matrix or a bead). In some embodiments, it is preferred to immobilize the polypeptides using a support compatible with the signal detection method, sensor, and/or device. In some examples, the preparation of the polypeptides including joining the polypeptides to the first detection agent may be performed prior to or after immobilizing the polypeptides. In some embodiments, a plurality of polypeptides are attached to a support prior to contacting the polypeptides with a binding agent.

In certain embodiments, a support is a bead, for example, a polystyrene bead, a polymer bead, a polyacrylate bead, an agarose bead, a cellulose bead, a dextran bead, an acrylamide bead, a solid core bead, a porous bead, a paramagnetic bead, a glass bead, a silica-based bead, or a controlled pore bead, or any combinations thereof. In some specific embodiments, the support is a porous agarose bead. In some embodiments, the support is a planar substrate. In some embodiments, the support is a bead array.

Various reactions may be used to attach the polypeptides to a support (e.g., a solid or a porous support). The polypeptides may be attached directly or indirectly to the support. In some cases, the polypeptides are attached to the support via a linker. Exemplary reactions include the copper catalyzed reaction of an azide and alkyne to form a triazole (Huisgen 1, 3-dipolar cycloaddition), strain-promoted azide alkyne cycloaddition (SPAAC), reaction of a diene and dienophile (Diels-Alder), strain-promoted alkyne-nitrone cycloaddition, reaction of a strained alkene with an azide, tetrazine or tetrazole, alkene and azide [3+2] cycloaddition, alkene and tetrazine inverse electron demand Diels-Alder (IEDDA) reaction (e.g., m-tetrazine (mTet) or phenyl tetrazine (pTet) and trans-cyclooctene (TCO)); or pTet and an alkene), alkene and tetrazole photoreaction, Staudinger ligation of azides and phosphines, and various displacement reactions, such as displacement of a leaving group by nucleophilic attack on an electrophilic atom (Horisawa 2014, Knall, Hollauf et al. 2014). Exemplary displacement reactions include reaction of an amine with: an activated ester; an N-hydroxysuccinimide ester; an isocyanate; an isothioscyanate, an aldehyde, an epoxide, or the like. In some embodiments, iEDDA click chemistry is used for immobilizing polypeptides to a support since it is rapid and delivers high yields at low input concentrations. In another embodiment, m-tetrazine rather than tetrazine is used in an iEDDA click chemistry reaction, as m-tetrazine has improved bond stability. In another embodiment, phenyl tetrazine (pTet) is used in an iEDDA click chemistry reaction. In one case, a polypeptide is labeled with a bifunctional click chemistry reagent, such as alkyne-NHS ester (acetylene-PEG-NETS ester) reagent or alkyne-benzophenone to generate an alkyne-labeled polypeptide. In some embodiments, an alkyne can also be a strained alkyne, such as cyclooctynes including Dibenzocyclooctyl (DBCO), etc.

In some embodiments, the support comprises a reacting agent. For example, the reacting agent comprises an azide group. In some cases, the polypeptide is linked to the support by reaction of an alkyline group in the trifunctional linker and an azide group present on the support.

In certain embodiments where multiple polypeptides are immobilized on the same support, the polypeptides can be spaced appropriately to accommodate methods of performing the binding reaction and any downstream detection and/or analysis steps to be used to assess the polypeptide. For example, it may be advantageous to space the molecules optimally for the signal detection step. In some cases, the appropriate spacing depends on the type of signal generated and detection method or sensor used to detect the signal. In some cases, spacing of the targets on the support is determined based on the consideration that a signal generated in association with one polypeptide may obscure or be indistinguishable with a signal generated with a neighboring molecule. In some embodiments, the polypeptides are immobilized on a support and spaced at optically resolvable distances.

In some embodiments, the surface of the support is passivated (blocked). A “passivated” surface refers to a surface that has been treated with outer layer of material. Methods of passivating surfaces include standard methods from the fluorescent single molecule analysis literature, including passivating surfaces with polymer like polyethylene glycol (PEG) (Pan et al., 2015, Phys. Biol. 12:045006), polysiloxane (e.g., Pluronic F-127), star polymers (e.g., star PEG) (Groll et al., 2010, Methods Enzymol. 472:1-18), hydrophobic dichlorodimethylsilane (DDS)+self-assembled Tween-20 (Hua et al., 2014, Nat. Methods 11:1233-1236), diamond-like carbon (DLC), DLC+PEG (Stavis et al., 2011, Proc. Natl. Acad. Sci. USA 108:983-988), and zwitterionic moiety (e.g., U.S. Patent Application Publication US 2006/0183863). In addition to covalent surface modifications, a number of passivating agents can be employed as well including surfactants like Tween-20, polysiloxane in solution (Pluronic series), poly vinyl alcohol (PVA), and proteins like BSA and casein. Alternatively, density of macromolecules (e.g., proteins, polypeptide, or peptides) can be titrated on the surface or within the volume of a solid substrate by spiking a competitor or “dummy” reactive molecule when immobilizing the proteins, polypeptides or peptides to the solid substrate.

To control spacing of the immobilized polypeptides on the support, the density of functional coupling groups for attaching the polypeptide (e.g., TCO or carboxyl groups (COOH)) and/or the first detection agent may be titrated on the substrate surface. In some embodiments, multiple molecules are spaced apart on the surface or within the volume (e.g., porous supports) of a support such that adjacent molecules are spaced apart at a distance of about 50 nm to about 500 nm, or about 50 nm to about 400 nm, or about 50 nm to about 300 nm, or about 50 nm to about 200 nm, or about 50 nm to about 100 nm. In some embodiments, multiple molecules are spaced apart on the surface of a support with an average distance of at least 50 nm, at least 60 nm, at least 70 nm, at least 80 nm, at least 90 nm, at least 100 nm, at least 150 nm, at least 200 nm, at least 250 nm, at least 300 nm, at least 350 nm, at least 400 nm, at least 450 nm, or at least 500 nm.

In some embodiments, appropriate spacing of the polypeptides and/or first detection agents on the support is accomplished by titrating the ratio of available attachment molecules on the substrate surface. In some examples, the substrate surface (e.g., bead surface) is functionalized with a carboxyl group (COOH) which is treated with an activating agent (e.g., activating agent is EDC and Sulfo-NHS). In some examples, the substrate surface (e.g., bead surface) comprises NHS moieties. In some embodiments, a mixture of mPEGₙ-NH₂ and NH₂-PEGₙ-mTet is added to the activated beads (wherein n is any number, such as 1-100). The ratio between the mPEG₃-NH₂ (not available for coupling) and NH₂-PEG₂₄-mTet (available for coupling) is titrated to generate an appropriate density of functional moieties available to attach the polypeptides on the substrate surface. In certain embodiments, the mean spacing between coupling moieties (e.g., NH₂-PEG₄-mTet) on the solid surface is at least 50 nm, at least 100 nm, at least 250 nm, or at least 500 nm. In some embodiments, the spacing of the polypeptides on the support is achieved by controlling the concentration and/or number of available COOH or other functional groups on the support.

Following the step of providing the polypeptide and an associated first detection agent joined to the solid support, the method further comprises the step of contacting the polypeptide with a binding agent capable of binding to the polypeptide, wherein the binding agent is associated with a second detection agent, whereby binding between the polypeptide and the binding agent brings the first detection agent and the second detection agent into sufficient proximity to generate a detectable label. A binding agent can be any molecule (e.g., peptide, polypeptide, protein, nucleic acid, carbohydrate, small molecule, and the like) capable of binding to a component or feature of a polypeptide. A binding agent can be a naturally occurring, synthetically produced, or recombinantly expressed molecule. In some embodiments, the scaffold used to engineer a binding agent can be from any species, e.g., human, non-human, transgenic. A binding agent may bind to a portion of a target macromolecule or a motif. A binding agent may bind to a single monomer or subunit of a polypeptide (e.g., a single amino acid) or bind to multiple linked subunits of a polypeptide (e.g., dipeptide, tripeptide, or higher order peptide of a longer polypeptide molecule).

In some embodiments, a binding agent is joined to a second detection agent via SpyCatcher-SpyTag interaction. The SpyTag peptide forms an irreversible covalent bond to the SpyCatcher protein via a spontaneous isopeptide linkage, thereby offering a genetically encoded way to create peptide interactions that resist force and harsh conditions (Zakeri et al., 2012, Proc. Natl. Acad. Sci. 109:E690-697; Li et al., 2014, J. Mol. Biol. 426:309-317). A binding agent may be expressed as a fusion protein comprising the SpyCatcher protein. In other embodiments, a binding agent is joined to a second detection agent via SnoopTag-SnoopCatcher peptide-protein interaction. The SnoopTag peptide forms an isopeptide bond with the SnoopCatcher protein (Veggiani et al., Proc. Natl. Acad. Sci. USA, 2016, 113:1202-1207). A binding agent may be expressed as a fusion protein comprising the SnoopCatcher protein. In yet other embodiments, a binding agent is joined to a second detection agent via the HaloTag® protein fusion tag and its chemical ligand. HaloTag is a modified haloalkane dehalogenase designed to covalently bind to synthetic ligands (HaloTag ligands) (Los et al., 2008, ACS Chem. Biol. 3:373-382). The synthetic ligands comprise a chloroalkane linker attached to a variety of useful molecules. A covalent bond forms between the HaloTag and the chloroalkane linker that is highly specific, occurs rapidly under physiological conditions, and is essentially irreversible. In some embodiments, a binding agent is joined to a second detection agent using a cysteine bioconjugation method. In some embodiments, a binding agent is joined to a second detection agent using 7c-clamp-mediated cysteine bioconjugation (See e.g., Zhang et al., Nat Chem. (2016) 8(2):120-128). In some cases, a binding agent is joined to a second detection agent using 3-arylpropiolonitriles (APN)-mediated tagging (e.g. Koniev et al., Bioconjug Chem. 2014; 25(2):202-206).

As illustrated in FIG. 1B, a cognate binding agent 200 is shown selectively binding to NTAA 210 of peptide 112. Cognate binding agent 200 is linked to first detection agent 204 through linker 216. Such selective binding of the cognate binding agent to the NTAA brings first detection agent 120 and second detection agent 204 into sufficient proximity, which generates a detectable signal. In FIG. 1C, when the peptide is contacted with non-cognate binding agent 202, which moiety is not capable of binding the NTAA 210 of peptide 112, the first detection agent 120 and second detection agent 204 are not in proximity, and thus no signal is generated.

The methods described herein use a binding agent capable of binding to the polypeptides. The binding reaction may be performed by contacting a single binding agent with a single polypeptide, a single binding agent with a plurality of polypeptides, a plurality of binding agents with a single polypeptide, or a plurality of binding agents to a plurality of polypeptides. In some embodiments, the plurality of binding agents includes a mixture of binding agents. In some embodiments that utilize a plurality of binding agents, the binding agent can be provided sequentially or simultaneously. In some embodiments, a plurality of one type of binding agent is contacted with the polypeptide, the signal or lack thereof is observed, and a plurality of another binding agent is contacted with the polypeptide. Various pools of binding agents can be contacted with the polypeptides in this manner sequentially. In some other embodiments, a pool of various binding agents are contacted with the polypeptides simultaneously. In some cases, each binding agent is associated with a second detection agent which may generate a different detectable signal or distinguishable detectable signal. In some examples, each of the second detection agents of the plurality of binding agents, when brought into sufficient proximity with the first detection agent, a detectable label is generated dependent on the identity of the target of the binding agent, to which each of the plurality of binding agents selectively bind. The signal generated by the label may also be dependent on the identity of the target of the binding agent.

In some examples, the binding agent comprises an antibody, an antigen-binding antibody fragment, a single-domain antibody (sdAb), a recombinant heavy-chain-only antibody (VHH), a single-chain antibody (scFv), a shark-derived variable domain (vNARs), a Fv, a Fab, a Fab′, a F(ab′)2, a linear antibody, a diabody, an aptamer, a peptide mimetic molecule, a fusion protein, a reactive or non-reactive small molecule, or a synthetic molecule.

In some examples, a plurality of binding agents are a plurality of aptamers, wherein each aptamer from the plurality of aptamers exhibits binding specificity toward at least one N-terminal amino acid residue of a polypeptide immobilized on a solid support. Generation of such aptamers are disclosed in US 20210079557 A1, incorporated herein by reference.

In certain embodiments, a binding agent may be designed to bind covalently. Covalent binding can be designed to be conditional or favored upon binding to the correct moiety. For example, an target and its cognate binding agent may each be modified with a reactive group such that once the target-specific binding agent is bound to the target, a coupling reaction is carried out to create a covalent linkage between the two. Non-specific binding of the binding agent to other locations that lack the cognate reactive group would not result in covalent attachment. In some embodiments, the polypeptide is capable of forming a covalent bond to a binding agent. In some embodiments, the target comprises a ligand group that is capable of covalent binding to a binding agent. Covalent binding between a binding agent and its target may allow for more stringent washing to be used to remove binding agents that are non-specifically bound, thus increasing the specificity of the assay. In some embodiment, the method further includes performing one or more wash steps. In some embodiments, the method includes a wash step after contacting the binding agent to the polypeptides to remove non-specifically bound binding agents. The stringency of the wash step may be tuned depending on the affinity of the binding agent to the polypeptides.

In some embodiments, the binding reaction involves binding agents configured to provide specificity for binding of the binding agent to the polypeptide. A binding agent may bind to an N-terminal peptide, a C-terminal peptide, or an intervening peptide of a peptide, polypeptide, or protein molecule. A binding agent may bind to an N-terminal amino acid, C-terminal amino acid, or an intervening amino acid of a peptide molecule. A binding agent may bind to an N-terminal or C-terminal diamino acid moiety. An N-terminal diamino acid is comprised of the N-terminal amino acid and the penultimate N-terminal amino acid. A C-terminal diamino acid is similarly defined for the C-terminus. A binding agent may preferably bind to a chemically modified or labeled amino acid. In certain embodiments, a binding agent may be a selective binding agent. As used herein, selective binding refers to the ability of the binding agent to preferentially bind to a specific ligand (e.g., amino acid or class of amino acids) relative to binding to a different ligand (e.g., amino acid or class of amino acids). Selectivity is commonly referred to as the equilibrium constant for the reaction of displacement of one ligand by another ligand in a complex with a binding agent. Typically, such selectivity is associated with the spatial geometry of the ligand and/or the manner and degree by which the ligand binds to a binding agent, such as by hydrogen bonding, hydrophobic binding, and Van der Waals forces (non-covalent interactions) or by reversible or non-reversible covalent attachment to the binding agent. It should also be understood that selectivity may be relative, and as opposed to absolute, and that different factors can affect the same, including ligand concentration. Thus, in one example, a binding agent selectively binds one of the twenty standard amino acids. In some examples, a binding agent binds to an N-terminal amino acid residue, a C-terminal amino acid residue, or an internal amino acid residue.

In some embodiments, the binding agent is partially specific or selective. In some aspects, the binding agent preferentially binds one or more amino acids. In some examples, a binding agent may bind to or is capable of binding to two or more of the twenty standard amino acids. For example, a binding agent may preferentially bind the amino acids A, C, and G over other amino acids. In some other examples, the binding agent may selectively or specifically bind more than one amino acid. In some aspects, the binding agent may also have a preference for one or more amino acids at the second, third, fourth, fifth, etc. positions from the terminal amino acid. In some cases, the binding agent preferentially binds to a specific terminal amino acid and a penultimate amino acid. For example, a binding agent may preferentially bind AA, AC, and AG or a binding agent may preferentially bind AA, CA, and GA. In some embodiments, a binding agent may exhibit flexibility and variability in target binding preference in some or all of the positions of the targets. In some examples, a binding agent may have a preference for one or more specific target terminal amino acids and have a flexible preference for a target at the penultimate position. In some other examples, a binding agent may have a preference for one or more specific target amino acids in the penultimate amino acid position and have a flexible preference for a target at the terminal amino acid position. In some embodiments, a binding agent is selective for a target comprising a terminal amino acid and other components of a macromolecule. In some examples, a binding agent is selective for a target comprising a terminal amino acid and at least a portion of the peptide backbone. In some particular examples, a binding agent is selective for a target comprising a terminal amino acid and an amide peptide backbone. In some cases, the peptide backbone comprises a natural peptide backbone or a post-translational modification. In some embodiments, the binding agent exhibits allosteric binding.

In some embodiments, the binding reaction comprises contacting a mixture of binding agents with a mixture of targets and selectively need only be relative to the other binding agents to which the target is exposed. It should also be understood that selectivity of a binding agent need not be absolute to a specific molecule but could be to a portion of a molecule. In some examples, selectivity of a binding agent need not be absolute to a specific amino acid, but could be selective to a class of amino acids, such as amino acids with polar or non-polar side chains, or with electrically (positively or negatively) charged side chains, or with aromatic side chains, or some specific class or size of side chains, and the like. In some embodiments, the ability of a binding agent to selectively bind a feature or component of a macromolecule is characterized by comparing binding abilities of binding agents. For example, the binding ability of a binding agent to the target can be compared to the binding ability of a binding agent which binds to a different target, for example, comparing a binding agent selective for a class of amino acids to a binding agent selective for a different class of amino acids. In some examples, a binding agent selective for non-polar side chains is compared to a binding agent selective for polar side chains. In some embodiments, a binding agent selective for a feature, component of a peptide, or one or more amino acid exhibits at least 1×, at least 2×, at least 5×, at least 10×, at least 50×, at least 100×, or at least 500× more binding compared to a binding agent selective for a different feature, component of a peptide, or one or more amino acid.

In some embodiments, binding between the binding agent and polypeptide or portion thereof is sufficient for the provided methods as long as it allows the first and second detection agents to be brought into sufficient proximity to generate a detectable label. In the practice of the methods disclosed herein, the ability of a cognate binding agent to selectively bind a particular NTAA need only be sufficient to generate a signal during the detecting step, or in the case of pooled contact, a signal distinguishable from other binding agents. In a particular embodiment, the binding agent has a high affinity and high selectivity for the macromolecule, e.g., the polypeptide, of interest. In particular, a high binding affinity with a low off-rate may be efficacious for the first and second detection agents to generate a detectable signal. In certain embodiments, a binding agent has a Kd of about <500 nM, <200 nM, <100 nM, <50 nM, <10 nM, <5 nM, <1 nM, <0.5 nM, or <0.1 nM. In a particular embodiment, the binding agent is added to the polypeptide at a concentration >1×, >5×, >10×, >100×, or >1000× its Kd to drive binding to completion. For example, binding kinetics of an antibody to a single protein molecule is described in Chang et al., J Immunol Methods (2012) 378(1-2): 102-115. In a particular embodiment, the provided methods for performing a binding reaction is compatible with a binding agent with medium to low affinity for the target macromolecule.

In certain embodiments, a binding agent may bind to a terminal amino acid of a peptide, an intervening amino acid, dipeptide (sequence of two amino acids), tripeptide (sequence of three amino acids), or higher order peptide of a peptide molecule. In some embodiments, each binding agent in a library of binding agents selectively binds to a particular amino acid, for example one of the twenty standard naturally occurring amino acids. The standard, naturally-occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). In some embodiments, the binding agent binds to an unmodified or native (e.g., natural) amino acid. In some examples, the binding agent binds to an unmodified or native dipeptide (sequence of two amino acids), tripeptide (sequence of three amino acids), or higher order peptide of a peptide molecule. A binding agent may be engineered for high affinity for a native or unmodified N-terminal amino acid (NTAA), high specificity for a native or unmodified NTAA, or both. In some embodiments, binding agents can be developed through directed evolution of promising affinity scaffolds using phage display.

In certain embodiments, a binding agent may bind to a post-translational modification of an amino acid. In some embodiments, a peptide comprises one or more post-translational modifications, which may be the same of different. The NTAA, CTAA, an intervening amino acid, or a combination thereof of a peptide may be post-translationally modified. Post-translational modifications to amino acids include acylation, acetylation, alkylation (including methylation), biotinylation, butyrylation, carbamylation, carbonylation, deamidation, deiminiation, diphthamide formation, disulfide bridge formation, eliminylation, flavin attachment, formylation, gamma-carboxylation, glutamylation, glycylation, glycosylation, glypiation, heme C attachment, hydroxylation, hypusine formation, iodination, isoprenylation, lipidation, lipoylation, malonylation, methylation, myristolylation, oxidation, palmitoylation, pegylation, phosphopantetheinylation, phosphorylation, prenylation, propionylation, retinylidene Schiff base formation, S-glutathionylation, S-nitrosylation, S-sulfenylation, selenation, succinylation, sulfination, ubiquitination, and C-terminal amidation (see, also, Seo and Lee, 2004, J. Biochem. Mol. Biol. 37:35-44).

In certain embodiments, a lectin is used as a binding agent for detecting the glycosylation state of a protein, polypeptide, or peptide. Lectins are carbohydrate-binding proteins that can selectively recognize glycan epitopes of free carbohydrates or glycoproteins. In certain embodiments, a binding agent can be an aptamer (e.g., peptide aptamer, DNA aptamer, or RNA aptamer), a peptoid, an antibody or a specific binding fragment thereof, an amino acid binding protein or enzyme, an antibody binding fragment, an antibody mimetic, a peptide, a peptidomimetic, a protein, or a polynucleotide (e.g., DNA, RNA, peptide nucleic acid (PNA), a gPNA, bridged nucleic acid (BNA), xeno nucleic acid (XNA), glycerol nucleic acid (GNA), or threose nucleic acid (TNA), or a variant thereof). As used herein, the terms antibody and antibodies are used in a broad sense, to include not only intact antibody molecules, for example but not limited to immunoglobulin A, immunoglobulin G, immunoglobulin D, immunoglobulin E, and immunoglobulin M, but also any immunoreactive component(s) of an antibody molecule or portion thereof that immuno-specifically bind to at least one epitope. An antibody may be naturally occurring, synthetically produced, or recombinantly expressed. An antibody may be a fusion protein. An antibody may be an antibody mimetic. Examples of antibodies include but are not limited to, Fab fragments, Fab′ fragments, F(ab)₂ fragments, single chain antibody fragments (scFv), miniantibodies, nanobodies, diabodies, crosslinked antibody fragments, Affibody™, nanobodies, single domain antibodies, DVD-Ig molecules, alphabodies, affimers, affitins, cyclotides, molecules, and the like. As with antibodies, nucleic acid and peptide aptamers that specifically recognize a macromolecule, e.g., a peptide or a polypeptide, can be produced using known methods. In yet another embodiment, a binding agent may be a modified aminopeptidase. In some embodiments, the binding agent may be a modified aminopeptidase that has been engineered to recognize a labeled amino acid.

A binding agent can be made by modifying naturally-occurring or synthetically-produced proteins by genetic engineering to introduce one or more mutations in the amino acid sequence to produce engineered proteins that bind to a specific component or feature of a polypeptide (e.g., NTAA, CTAA, or post-translationally modified amino acid or a peptide). For example, exopeptidases (e.g., aminopeptidases, carboxypeptidases), exoproteases, mutated exoproteases, mutated anticalins, mutated ClpSs, antibodies, or tRNA synthetases can be modified to create a binding agent that selectively binds to a particular NTAA. Generation of protein-based specific NTAA binding agents are disclosed in U.S. Pat. No. 9,435,810 B2, WO 2020/223000 and provisional U.S. application 63/085,977. In another example, carboxypeptidases can be modified to create a binding agent that selectively binds to a particular CTAA. A binding agent can also be designed or modified, and utilized, to specifically bind a modified NTAA or modified CTAA, for example one that has a post-translational modification (e.g., phosphorylated NTAA or phosphorylated CTAA) or one that has been modified with a label (e.g., a chemical reagent). Strategies for directed evolution of proteins are known in the art (e.g., Yuan et al., 2005, Microbiol. Mol. Biol. Rev. 69:373-392), and include phage display, ribosomal display, mRNA display, CIS display, CAD display, emulsions, cell surface display method, yeast surface display, bacterial surface display, etc.

In some embodiments, a binding agent may bind to a native or unmodified or unlabeled terminal amino acid. Moreover, in some cases, these natural amino acid binders don't recognize N-terminal labels. Directed evolution of aaRS scaffolds can be used to generate higher affinity, higher specificity binding agents that recognized the N-terminal amino acids in the context of an N-terminal label. In another example, Havranak et al. (U.S. Patent Publication No. US 2014/0273004) describes engineering aminoacyl tRNA synthetases (aaRSs) as specific NTAA binders. The amino acid binding pocket of the aaRSs has an intrinsic ability to bind cognate amino acids, but generally exhibits poor binding affinity and specificity. Moreover, these natural amino acid binders don't recognize N-terminal labels. Directed evolution of aaRS scaffolds can be used to generate higher affinity, higher specificity binding agents that recognized the N-terminal amino acids in the context of an N-terminal label.

In some embodiments, a binding agent that selectively binds to a labeled, modified, or functionalized NTAA can be utilized. In some cases, the NTAA is modified by a chemical reagent prior to binding to the binding agent. A binding agent may be engineered for high affinity for a modified NTAA, high specificity for a modified NTAA, or both. In some embodiments, binding agents can be developed through directed evolution of promising affinity scaffolds using phage display.

For example, a polypeptide can be modified/functionalized before the step of contacting the polypeptide with the binding agent. In some cases, the polypeptide can be modified/functionalized after detecting the signal generated by the detectable label, prior to repeating the step of contacting the polypeptide with another cycle of binding agent(s). In some embodiments, a binding agent may bind to a chemically or enzymatically modified terminal amino acid. In some embodiments, the polypeptide or a portion thereof is labeled with a reagent selected from the group consisting of a phenyl isothiocyanate (PITC), a nitro-PITC, a sulfo-PITC, a phenyl isocyanate (PIC), a nitro-PIC, a sulfo-PIC, benzyloxycarbonyl chloride or carbobenzoxy chloride (Cbz-Cl), N-(Benzyloxycarbonyloxy)succinimide (Cbz-OSu or Cbz-O—NHS), a carboxyl-activated amino-blocked amino acid (e.g. Cbz-amino acid-OSu), a 1-fluoro-2,4-dinitrobenzene (Sanger's reagent, DNFB), dansyl chloride (DNS-Cl, or 1-dimethylaminonaphthalene-5-sulfonyl chloride), 4-sulfonyl-2-nitrofluorobenzene (SNFB), an anhydride, 2-Pyridinecarboxaldehyde, 2-Formylphenylboronic acid, 2-Acetylphenylboronic acid, 1-Fluoro-2,4-dinitrobenzene, 4-Chloro-7-nitrobenzofurazan, Pentafluorophenylisothiocyanate, 4-(Trifluoromethoxy)-phenylisothiocyanate, 4-(Trifluoromethyl)-phenylisothiocyanate, 3-(Carboxylic acid)-phenylisothiocyanate, 3-(Trifluoromethyl)-phenylisothiocyanate, 1-Naphthylisothiocyanate, N-nitroimidazole-1-carboximidamide, N,N′-Bis(pivaloyl)-1H-pyrazole-1-carboxamidine, N,N′-Bis(benzyloxycarbonyl)-1H-pyrazole-1-carboxamidine, an acetylating reagent, a guanidinylation reagent, a thioacylation reagent, a thioacetylation reagent, a thiobenzylation reagent, a diheterocyclic methanimine reagent, or a derivative thereof. In some embodiments, the polypeptide is labeled with an anhydride or derivative thereof. In some examples, the binding agent binds an amino acid labeled by contacting with a reagent or using a method as described in International Patent Publication No. WO 2019/089846 or International Patent Application No. PCT/US20/29969. In some cases, the binding agent binds an amino acid labeled by an amine modifying reagent. In some embodiments, the binding agent binds to a chemically modified N-terminal amino acid residue or a chemically modified C-terminal amino acid residue.

In a particular embodiment, anticalins are engineered for both high affinity and high specificity to labeled NTAAs (e.g. PTC, modified-PTC, Cbz, DNP, SNP, acetyl, guanidinyl, amino guanidinyl, heterocyclic methanimine, etc.). Certain varieties of anticalin scaffolds have suitable shape for binding single amino acids, by virtue of their beta barrel structure. An N-terminal amino acid (either with or without modification) can potentially fit and be recognized in this “beta barrel” bucket. High affinity anticalins with engineered novel binding activities have been described (reviewed by Skerra, 2008, FEBS J. 275: 2677-2683). For example, anticalins with high affinity binding (low nM) to fluorescein and digoxygenin have been engineered (Gebauer et al., 2012, Methods Enzymol 503: 157-188.). Engineering of alternative scaffolds for new binding functions has also been reviewed by Banta et al. (2013, Annu. Rev. Biomed. Eng. 15:93-113).

In some embodiments, a binding agent can be utilized that selectively binds a modified C-terminal amino acid (CTAA). Carboxypeptidases are proteases that cleave/eliminate terminal amino acids containing a free carboxyl group. A number of carboxypeptidases exhibit amino acid preferences, e.g., carboxypeptidase B preferentially cleaves at basic amino acids, such as arginine and lysine. A carboxypeptidase can be modified to create a binding agent that selectively binds to particular amino acid. In some embodiments, the carboxypeptidase may be engineered to selectively bind both the modification moiety as well as the alpha-carbon R group of the CTAA. Thus, engineered carboxypeptidases may specifically recognize 20 different CTAAs representing the standard amino acids in the context of a C-terminal label. Control of the stepwise degradation from the C-terminus of the peptide is achieved by using engineered carboxypeptidases that are only active (e.g., binding activity or catalytic activity) in the presence of the label. In one example, the CTAA may be modified by a para-Nitroanilide or 7-amino-4-methylcoumarinyl group.

Other potential scaffolds that can be engineered to generate binding agents for use in the methods described herein include: an anticalin, a lipocalin, an amino acid tRNA synthetase (aaRS), ClpS, an Affilin®, an Adnectin™, a T cell receptor, a zinc finger protein, a thioredoxin, GST A1-1, DARPin, an affimer, an affitin, an alphabody, an avimer, a monobody, an antibody, a single domain antibody, a nanobody, EETI-II, HPSTI, intrabody, PHD-finger, V(NAR) LDTI, evibody, Ig(NAR), knottin, maxibody, microbody, neocarzinostatin, pVIII, tendamistat, VLR, protein A scaffold, MTI-II, ecotin, GCN4, Im9, kunitz domain, PBP, trans-body, tetranectin, WW domain, CBM4-2, DX-88, GFP, iMab, Ldl receptor domain A, Min-23, PDZ-domain, avian pancreatic polypeptide, charybdotoxin/10Fn3, domain antibody (Dab), a2p8 ankyrin repeat, insect defensing A peptide, Designed AR protein, C-type lectin domain, staphylococcal nuclease, Src homology domain 3 (SH3), or Src homology domain 2 (SH2). See e.g., El-Gebali et al., (2019) Nucleic Acids Research 47:D427-D432 and Finn et al., (2013) Nucleic Acids Res. 42(Database issue):D222-D230. In some embodiments, a binding agent is derived from an enzyme which binds one or more amino acids (e.g., an aminopeptidase). In certain embodiments, a binding agent can be derived from an anticalin or a Clp protease adaptor protein (ClpS).

The functional affinity (avidity) of a given monovalent binding agent may be increased by at least an order of magnitude by using a bivalent or higher order multimer of the monovalent binding agent (Vauquelin et al., 2013, Br J Pharmacol 168(8): 1771-1785. 2013). In some embodiments, the binding agent is linked, directly or indirectly, to a multimerization domain. Thus, monomeric, dimeric, and higher order (e.g., 3, 4, 5, or more) multimeric polypeptides comprising one or more binding agents are provided herein. In some specific embodiments, the binding agent is dimeric. In some examples, two polypeptides can be covalently or non-covalently attached to each other to form a dimer.

In some embodiments, the binding agent is derived from a biological, naturally occurring, non-naturally occurring, or synthetic source. In some examples, the binding agent is derived from de novo protein design (Huang et al., (2016) 537(7620):320-327). In some examples, the binding agent has a structure, sequence, and/or activity designed from first principles.

A binding agent may preferably bind to a modified or labeled amino acid, by chemical or enzymatic means, (e.g., an amino acid that has been functionalized by a reagent (e.g., a compound)) over a non-modified or unlabeled amino acid. For example, a binding agent may preferably bind to an amino acid that has been functionalized with an acetyl moiety, Cbz moiety, guanyl moiety, dansyl moiety, PTC moiety, DNP moiety, SNP moiety, heterocyclic methanimine moiety, etc., over an amino acid that does not possess said moiety. In some embodiments, a binding agent may preferably bind to an amino acid that has been functionalized or modified as described in International Patent Publication No. WO 2019/089846. In some cases, a binding agent may bind to a post-translationally modified amino acid. Thus, in certain embodiments, a signal generated by the detectable label relating to amino acid sequence may also include information regarding post-translational modifications of the polypeptide. Once the detection of the generated signal is complete, the PTM modifying groups can be removed. In some embodiments, the PTM modifying groups can be removed prior to contacting the binding agent with the polypeptide.

In certain embodiments, a polypeptide is also contacted with a non-cognate binding agent. As used herein, a non-cognate binding agent is referring to a binding agent that is selective for a different target (e.g. polypeptide feature or component) than the particular target being considered. For example, if the n NTAA is phenylalanine, and the peptide is contacted with three binding agents selective for phenylalanine, tyrosine, and asparagine, respectively, the binding agent selective for phenylalanine would be first binding agent capable of selectively binding to the NTAA (i.e., phenylalanine), while the other two binding agents would be non-cognate binding agents for that peptide (since they are selective for NTAAs other than phenylalanine). The tyrosine and asparagine binding agents may, however, be cognate binding agents for other peptides in the sample. If the n NTAA (phenylalanine) was then cleaved from the peptide, thereby converting the n−1 amino acid of the peptide to the n−1 NTAA (e.g., tyrosine), and the peptide was then contacted with the same three binding agents, the binding agent selective for tyrosine would be second binding agent capable of selectively binding to the n−1 NTAA (i.e., tyrosine), while the other two binding agents would be non-cognate binding agents (since they are selective for NTAAs other than tyrosine).

Thus, it should be understood that whether an agent is a binding agent or a non-cognate binding agent will depend on the nature of the particular polypeptide feature or component currently available for binding. Also, if multiple polypeptides are analyzed in a multiplexed reaction, a binding agent for one polypeptide may be a non-cognate binding agent for another, and vice versa. According, it should be understood that the following description concerning binding agents is applicable to any type of binding agent described herein (i.e., both cognate and non-cognate binding agents).

In certain embodiments, the concentration of the binding agents in a solution is controlled to reduce background and/or false positive results of the assay.

In some embodiments, the concentration of a binding agent can be at any suitable concentration, e.g., at about 0.0001 nM, about 0.001 nM, about 0.01 nM, about 0.1 nM, about 1 nM, about 2 nM, about 5 nM, about 10 nM, about 20 nM, about 50 nM, about 100 nM, about 200 nM, about 500 nM, or about 1,000 nM. In other embodiments, the concentration of a soluble conjugate used in the assay is between about 0.0001 nM and about 0.001 nM, between about 0.001 nM and about 0.01 nM, between about 0.01 nM and about 0.1 nM, between about 0.1 nM and about 1 nM, between about 1 nM and about 2 nM, between about 2 nM and about 5 nM, between about 5 nM and about 10 nM, between about 10 nM and about 20 nM, between about 20 nM and about 50 nM, between about 50 nM and about 100 nM, between about 100 nM and about 200 nM, between about 200 nM and about 500 nM, between about 500 nM and about 1000 nM, or more than about 1,000 nM.

In some embodiments, the ratio between the soluble binding agent molecules and the immobilized polypeptides can be at any suitable range, e.g., at about 0.00001:1, about 0.0001:1, about 0.001:1, about 0.01:1, about 0.1:1, about 1:1, about 2:1, about 5:1, about 10:1, about 15:1, about 20:1, about 25:1, about 30:1, about 35:1, about 40:1, about 45:1, about 50:1, about 55:1, about 60:1, about 65:1, about 70:1, about 75:1, about 80:1, about 85:1, about 90:1, about 95:1, about 100:1, about 10⁴:1, about 10⁵:1, about 10⁶:1, or higher, or any ratio in between the above listed ratios. Higher ratios between the soluble binding agent molecules and the immobilized polypeptide(s) and/or the nucleic acids can be used to drive the binding. This may be particularly useful for detecting and/or analyzing low abundance polypeptides in a sample.

Following the step of contacting the peptide with the binding agent associated with a second detection agent, the signal generated by the detectable label is detected. In some embodiments, the step includes observing the lack of or absence of signal generated by the detectable label. In some embodiments, the signal is generated by a detectable label formed by joining of the first and second detection agents. In some cases, the signal may be generated by a detectable label formed by the first detection agent in the presence of the second detection agent, or by a detectable label formed by the second detection agent in the presence of the first detection agent. Detection or observation of such a signal may be accomplished by any number of known techniques. Such monitoring may be direct or indirect, and includes both chemical and/or optical techniques. The appropriate detection technique and sensors can be selected based on the detection agents used. In some embodiments, the detection includes chemical detection or optical detection. In some cases, the detection includes detecting a change in pH. For example, the change in pH is the result of a release of protons (H+). In some embodiments, wherein the signal generated is luminescent-based. In some embodiments, the signal generated is fluorescent-based.

Representative techniques include fluorescence polarization, fluorescence intensity, fluorescence lifetime, fluorescence energy transfer, pH, ionic content, temperature or combinations thereof. In the case of monitoring change in pH, such change can result from the release of protons (H⁺). In some embodiments, the signal generated by the detectable label is the release of protons. In the case of monitoring fluorescence, release of photons may be observed. In some embodiments, fluorescence and/or photon release may be catalyzed by an additional enzyme distinct from the first and/or second detection agents. For example, ATP sulfurylase converts a released PPi to ATP in the presence of adenosine 5′ phosphosulfate. This ATP acts as a substrate for luciferase-mediated conversion of luciferin to oxyluciferin that generates visible light in amounts that are proportional to the amount of ATP. The light produced in the luciferase-catalyzed reaction can be detected by a suitable device.

Such monitoring of the signal generated by the detectable label can be performed on any number of commercially available devices. For example, the signal may be read by a field effect transistor (FET). Moreover, existing devices may be modified or adapted for use in the methods of the present invention. The appropriate device can be selected or modified based on the signal produced in the assays of the present invention. In an example where the signal is proton release, which results in a detectable pH change, a suitable device may be the Ion Torrent PGM and Proton machine. The Ion Torrent device uses a change in charge (proton release and/or pH drop) to generate a measurable, electrical signal. The Ion Torrent platform uses a disposable chip that is built using semiconductor technology. In an example where the signal is photon release, a suitable device may be the 454 Life Sciences instrument, which uses a coupled sulfurylase-luciferase enzymatic reaction to generate a photon. In an example where the signal is generated by a fluorescent protein or a split fluorescent protein, a suitable device may utilize optical detection (e.g., fluorescence detection) to generate a measurable signal. These devices also permit massive multiplexing for the digital detection, analysis and sequencing of more than 100 million protein molecules in a single assay.

The detection agents or detectable labels as described in the methods of the present invention can be detected by any means known in the art. The detection can be direct or indirect detection. The detection can be chemical detection or optical detection. The detection can be a detection of fluorescence polarization, fluorescence intensity, fluorescence lifetime, fluorescence energy transfer, pH, ionic content, temperature or combinations thereof. The detection can be a detection of a change in pH. The change in pH can be the result of a release of protons (H+). The detection can be a detection of photons. The detection can be a detection of fluorescence. The detection can identify the N-terminal amino acid of the peptide.

In some embodiments for detection utilizing a split protein or split enzyme system, fluorescence and/or photon release may be catalyzed by an additional enzyme distinct from the first and second detection agents. For example, ATP sulfurylase converts a released PPi to ATP in the presence of adenosine 5′ phosphosulfate. This ATP acts as a substrate for luciferase-mediated conversion of luciferin to oxyluciferin that generates visible light in amounts that are proportional to the amount of ATP. The light produced in the luciferase-catalyzed reaction can be detected by a suitable device.

The detection of signal in the assays of the present invention can be performed on many commercially available devices. Moreover, existing devices may be modified or adapted for use in the methods of the present invention. The appropriate device can be selected or modified based on the signal produced in the assays of the present invention.

In an example where the signal is proton release, which results in a detectable pH change, a suitable device may be the Ion Torrent PGM and Proton machine. The Ion Torrent device uses a change in charge (proton release and/or pH drop) to generate a measurable, electrical signal. The Ion Torrent platform uses a disposable chip that is built using semiconductor technology. In an example where the signal is photon release, a suitable device may be the 454 Life Sciences instrument, which uses a coupled sulfurylase-luciferase enzymatic reaction to generate a photon. In an example where the signal is generated by a fluorescent protein or a split fluorescent protein, a suitable device may utilize optical detection (e.g., fluorescence detection) to generate a measurable signal. These devices also permit massive multiplexing for the digital detection, analysis and sequencing of more than 100 million protein molecules in a single assay.

In some embodiments, the signal generated by the detectable label is quenched or deactivated after the detection. In some embodiments, the signal generated by the detectable label is quenched or deactivated before contacting the polypeptide with additional binding agents. In some cases, the method includes releasing the second detection agent from the first detection agent after the detection. For example, the binding agent is released from the polypeptide after detection and/or prior to repeating the step of providing one or more binding agents.

### II. CYCLIC DETECTION METHOD AND APPLICATIONS

Provided in the methods herein, following one cycle of contacting the polypeptides with binding agents and signal detection, these steps may be repeated sequentially one or more times. In some embodiments, the step of contacting the polypeptides with a binding agent comprises contacting the polypeptides with a plurality of binding agents as a mixture; each binding agent is joined to a different second detection agent; and the signal generated by the detectable label is different for each binding agent. In some embodiments, in each cycle during the contacting step a polypeptide is contacted with a different binding agent that is joined to the same second detection agent. In some embodiments, in each cycle during the contacting step a polypeptide is contacted with the same plurality of binding agents, wherein each binding agent of the plurality of binding agents is joined to a different second detection agent.

In some embodiments, the method further includes removing a portion of the polypeptide. In some embodiments, the method includes removing the terminal amino acid from the peptide, thereby yielding a newly exposed terminal amino acid, and contacting with a binding agent may be repeated on the newly exposed terminal amino acid. Removal of a portion of the polypeptide, e.g., a terminal amino acid such as a NTAA, may be accomplished by any number of known techniques, including chemical and enzymatic techniques. In some embodiments, the repeated steps for analyzing the newly exposed NTAA are substantially similar to the first cycle, including contacting with a binding agent capable of binding to the newly exposed NTAA and associated with a second detection agent, and detecting the signal generated by the detectable label formed when binding of the newly exposed NTAA by the binding agent brings the first detection agent and the second detection agent into sufficient proximity. In some cases, it may be beneficial to wash the polypeptide with, for example, a suitable buffer to remove and/or dissociate components between steps.

A. Cyclic Detection

Provided herein is method for analyzing a polypeptide, comprising (a) providing a polypeptide and an associated first detection agent joined to a support; (b) contacting the polypeptide with a binding agent capable of binding to the polypeptide, wherein the binding agent is associated with a second detection agent, whereby binding between the polypeptide and the binding agent brings the first detection agent and the second detection agent into sufficient proximity to interact with each other and generate a detectable label; (c) detecting a signal generated by the detectable label; and optionally (d) removing a portion of the polypeptide. In some embodiments, step (b), (c), and (d) are sequentially repeated one or more times. In some embodiments, the portion of the polypeptide is removed with a bound binding agent. In some embodiments, a portion of the polypeptide removed includes a terminal amino acid. In some examples, the removal is performed by contacting the polypeptide with a chemical or enzymatic reagent.

In some particular embodiments, the method further includes contacting the polypeptide with a reagent for modifying a terminal amino acid. For example, the polypeptide is contacting with a reagent for modifying the terminal amino acid prior to step (d) removing the portion of the polypeptide. In some cases, the polypeptide is contacted with the reagent for modifying a terminal amino acid prior to step (b). In some cases, the polypeptide is contacted with the reagent for modifying a terminal amino acid after step (c).

In some embodiments, some of the steps (b), (c), and (d) can be performed in various orders. In one example, the polypeptide(s) is treated with the reagent for modifying a terminal amino acid of the polypeptide, followed by being contacted with the binding agent, followed by detecting the signal generated by the first and/or second detection agents, followed by removal of a portion of the polypeptide. In some cases, the polypeptide(s) is contacted with the binding agent, followed by detecting the signal generated by the first and/or second detection agents, followed by treating with the reagent for modifying a terminal amino acid of the polypeptide, followed by removal of a portion of the polypeptide.

In some embodiments, the first detection agent is removed with a portion of the polypeptide. In some cases, the portion of the polypeptide removed comprises the N-terminal amino acid, thereby yielding a newly exposed NTAA of the polypeptide. In some cases, the chemical or enzymatic reagent selectively removes the N-terminal amino acid (NTAA) of the polypeptide. In some cases, the NTAA is modified or functionalized by a chemical reagent prior to removal. In some embodiments, one amino acid is removed from the polypeptide. In some other embodiments, two amino acids are removed from the polypeptide. In some of any such embodiments, the amino acid is removed from the polypeptide by a chemical cleavage or an enzymatic cleavage.

In some embodiments, the removal of the portion of the polypeptide also removes or dissociates the first detection agent associated from the polypeptide. In some such embodiments, the method further includes providing the polypeptides with the first detection agent after step (d), e.g. after the NTAA is removed from the polypeptide.

In one exemplary cyclic workflow, the polypeptides comprising NTAAs may be contacted with the cognate and non-cognate binding agents in simultaneous or pooled manner. The size of the pool may vary, and a plurality of binding agents may be employed, wherein the plurality comprises binding agents capable of selectively binding at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or each of the 20 amino acids simultaneously. In one embodiment, the plurality of binding agents can comprise binding agents which can competitively bind to a class or group of amino acids. In this embodiment, the nature of the signal generated may be unique or different between the various cognate and non-cognate binding agents, such that the nature of the signal identifies which of the plurality of binding agents selectively bound to the NTAA.

In one embodiment, the plurality of peptides may be analyzed by decoding through repeated cycles of pools of binding agents combinatorially-labeled with the second detection agent. In a first cycle of decoding, a subset of NTAAs associated with the plurality of peptides are detected in a “lighted” state by contact with cognate binding agents having second detection agents (labeled cognate binding agents), while at the same time a subset of NTAAs associated with the plurality of peptides are detected in a “dark” state by contact with cognate binding agents lacking the second detection agents (unlabeled cognate binding agents). In this way, contact with the labeled cognate binding agents generate a distinguishable signal relative to a subset of unlabeled cognate binding agents. Repeated cycles generate a binary code representing the signal across the decoding cycles (See e.g., Gunderson et al. Genome Research, 14:870-877, 2004).

In some embodiments, the method further includes removing the binding agent after detecting the signal generated by the first and/or second detection agents. In some aspects, the binding agent is removed after detecting the signal generated by the first and/or second detection agents and before repeating the step of providing the polypeptide with a binding agent.

In embodiments relating to methods of analyzing target peptides or polypeptides using a degradation based approach, following contacting and binding of a first binding agent to an n NTAA of a peptide of n amino acids and detecting the signal generated, the n NTAA is eliminated. Removal of the n labeled NTAA by contacting with an enzyme or chemical reagents converts the n−1 amino acid of the peptide to an N-terminal amino acid, which is referred to herein as an n−1 NTAA. A second binding agent is contacted with the peptide and binds to the n−1 NTAA, and the signal generated is detected. In some embodiments, a signal or a lack of signal generated by the detectable label is observed and/or detected. Elimination of the n−1 labeled NTAA converts the n−2 amino acid of the peptide to an N-terminal amino acid, which is referred to herein as n−2 NTAA. Additional binding and detection can occur as described above up to n amino acids, wherein the observed signals over two or more cycles collectively represent the peptide. As used herein, an n “order” when used in reference to a binding agent refers to the n binding cycle. In some embodiments, one or more wash steps are performed before, within, or after each cycle. In some embodiments, steps including the NTAA in the described exemplary approach can be performed instead with a C terminal amino acid (CTAA).

In certain embodiments relating to analyzing peptides, following binding of a terminal amino acid (N-terminal or C-terminal) by a binding agent and detecting the signal generated by the first and/or second detection agents, the terminal amino acid is removed or cleaved from the peptide to expose a new terminal amino acid. In some embodiments, the terminal amino acid is an NTAA. In other embodiments, the terminal amino acid is a CTAA. Cleavage of a terminal amino acid can be accomplished by any number of known techniques, including chemical cleavage and enzymatic cleavage. In some embodiments, an engineered enzyme that catalyzes or reagent that promotes the removal of the PITC-derivatized or other labeled N-terminal amino acid is used. In some embodiments, the terminal amino acid is removed or eliminated using any of the methods as described in US 2020/0348307 A1, WO 2020/223133 or WO 2020/198264 A1. In some embodiments, cleavage of a terminal amino uses a carboxypeptidase, an aminopeptidase, a dipeptidyl peptidase, a dipeptidyl aminopeptidase or a variant, mutant, or modified protein thereof; a hydrolase or a variant, mutant, or modified protein thereof; a mild Edman degradation reagent; an Edmanase enzyme; anhydrous TFA, a base; or any combination thereof. In some embodiments, the mild Edman degradation uses a dichloro or monochloro acid; the mild Edman degradation uses TFA, TCA, or DCA; or the mild Edman degradation uses triethylamine, triethanolamine, or triethylammonium acetate (Et₃NHOAc). In some cases, the reagent for removing the amino acid comprises a base. In some embodiments, the base is a hydroxide, an alkylated amine, a cyclic amine, a carbonate buffer, trisodium phosphate buffer, or a metal salt.

In some embodiments, the chemical reagent for removing a portion of the polypeptide is selected from the group consisting of a phenyl isothiocyanate (PITC), a nitro-PITC, a sulfo-PITC, a phenyl isocyanate (PIC), a nitro-PIC, a sulfo-PIC, Cbz-Cl (benzyl chloroformate) or Cbz-OSu (benzyloxycarbonyl N-succinimide), an anhydride, a 1-fluoro-2,4-dinitrobenzene (Sanger's reagent, DNFB), dansyl chloride (DNS-Cl, or 1-dimethylaminonaphthalene-5-sulfonyl chloride), 4-sulfonyl-2-nitrofluorobenzene (SNFB), 2-Pyridinecarboxaldehyde, 2-Formylphenylboronic acid, 2-Acetylphenylboronic acid, 1-Fluoro-2,4-dinitrobenzene, 4-Chloro-7-nitrobenzofurazan, Pentafluorophenylisothiocyanate, 4-(Trifluoromethoxy)-phenylisothiocyanate, 4-(Trifluoromethyl)-phenylisothiocyanate, 3-(Carboxylic acid)-phenylisothiocyanate, 3-(Trifluoromethyl)-phenylisothiocyanate, 1-Naphthylisothiocyanate, N-nitroimidazole-1-carboximidamide, N,N′-Bis(pivaloyl)-1H-pyrazole-1-carboxamidine, N,N′-Bis(benzyloxycarbonyl)-1H-pyrazole-1-carboxamidine, an acetylating reagent, a guanidinylation reagent, a thioacylation reagent, a thioacetylation reagent, a thiobenzylation reagent, and a diheterocyclic methanimine reagent, or a derivative thereof.

Enzymatic cleavage of a NTAA may be accomplished by a peptidase, e.g., a carboxypeptidase, aminopeptidase, or dipeptidyl peptidase, dipeptidyl aminopeptidase, or variant, mutant, or modified protein thereof. Aminopeptidases naturally occur as monomeric and multimeric enzymes, and may be metal or ATP-dependent. Natural aminopeptidases have very limited specificity, and generically cleave N-terminal amino acids in a processive manner, cleaving one amino acid off after another. For the methods described here, aminopeptidases (e.g., metalloenzymatic aminopeptidase) may be engineered to possess specific binding or catalytic activity to the NTAA only when modified with an N-terminal label. For example, an aminopeptidase may be engineered such than it only cleaves an N-terminal amino acid if it is modified by a group such as PTC, modified-PTC, Cbz, DNP, SNP, acetyl, guanidinyl, diheterocyclic methanimine, etc. In this way, the aminopeptidase cleaves only a single amino acid at a time from the N-terminus, and allows control of the degradation cycle. In some embodiments, the modified aminopeptidase is non-selective as to amino acid residue identity while being selective for the N-terminal label. In other embodiments, the modified aminopeptidase is selective for both amino acid residue identity and the N-terminal label.

Engineered aminopeptidase mutants that bind to and cleave individual or small groups of labelled (biotinylated) NTAAs have been described (see, PCT Publication No. WO2010/065322, incorporated by reference in its entirety). Aminopeptidases are enzymes that cleave amino acids from the N-terminus of proteins or peptides. Natural aminopeptidases have very limited specificity, and generically eliminate N-terminal amino acids in a processive manner, cleaving one amino acid off after another (Kishor et al., 2015, Anal. Biochem. 488:6-8). However, residue specific aminopeptidases have been identified (Eriquez et al., J. Clin. Microbiol. 1980, 12:667-71; Wilce et al., 1998, Proc. Natl. Acad. Sci. USA 95:3472-3477; Liao et al., 2004, Prot. Sci. 13:1802-10). Aminopeptidases may be engineered to specifically bind to 20 different NTAAs representing the standard amino acids that are labeled with a specific moiety (e.g., PTC, DNP, SNP, etc.). Control of the stepwise degradation of the N-terminus of the peptide is achieved by using engineered aminopeptidases that are only active (e.g., binding activity or catalytic activity) in the presence of the label. In another example, Havranak et al. (U.S. Patent Publication No. US 2014/0273004) describes engineering aminoacyl tRNA synthetases (aaRSs) as specific NTAA binders. The amino acid binding pocket of the aaRSs has an intrinsic ability to bind cognate amino acids, but generally exhibits poor binding affinity and specificity. Moreover, these natural amino acid binders don't recognize N-terminal labels. Directed evolution of aaRS scaffolds can be used to generate higher affinity, higher specificity binding agents that recognized the N-terminal amino acids in the context of an N-terminal label.

For embodiments relating to CTAA binding agents, methods of cleaving CTAA from peptides are also known in the art. For example, U.S. Pat. No. 6,046,053 discloses a method of reacting the peptide or protein with an alkyl acid anhydride to convert the carboxy-terminal into oxazolone, liberating the C-terminal amino acid by reaction with acid and alcohol or with ester. Enzymatic cleavage of a CTAA may also be accomplished by a carboxypeptidase. Several carboxypeptidases exhibit amino acid preferences, e.g., carboxypeptidase B preferentially cleaves at basic amino acids, such as arginine and lysine. As described above, carboxypeptidases may also be modified in the same fashion as aminopeptidases to engineer carboxypeptidases that specifically bind to CTAAs having a C-terminal label. In this way, the carboxypeptidase cleaves only a single amino acid at a time from the C-terminus, and allows control of the degradation cycle. In some embodiments, the modified carboxypeptidase is non-selective as to amino acid residue identity while being selective for the C-terminal label. In other embodiments, the modified carboxypeptidase is selective for both amino acid residue identity and the C-terminal label.

In some embodiments, the polypeptide is contacted with one or more additional enzymes to eliminate the NTAA (e.g., a proline aminopeptidase to remove an N-terminal proline, if present). In some embodiments, the enzyme eliminates an NTAA from the polypeptide that is a proline. In some specific examples, the enzyme is a proline aminopeptidase, a proline iminopeptidase (PIP), or a pyroglutamate aminopeptidase (pGAP). In some embodiments, the enzymes to treat the polypeptides can be used in combination with a chemical or enzymatic methods for removing/eliminating amino acids from the polypeptide. In some cases, enzymes can be provided as a cocktail. PAP enzymes that cleave N-terminal prolines are also referred to as proline iminopeptidases (PIPs). Known monomeric PAPs include family members from B. coagulans, L. delbrueckii, N. gonorrhoeae, F. meningosepticum, S. marcescens, T. acidophilum, L. plantarum (MEROPS 533.001) Nakajima et al., J Bacteriol. (2006) 188(4):1599-606; Kitazono et al., Bacteriol (1992) 174(24):7919-7925). Known multimeric PAPs including D. hansenii (Bolumar et al., (2003) 86(1-2):141-151) and similar homologues from other species (Basten et al., Mol Genet Genomics (2005) 272(6):673-679). Either native or engineered variants/mutants of PAPs may be employed.

In some instances, the information from the provided methods can be stored, analyzed, and/or determined using a software tool. The software may utilize information about the binding characteristics of each binding agent. The software could also utilize a listing of some or all spatial locations in which each a signal was generated or not generated by the detectable label. In some embodiments, the software may comprise a database. The database may contain sequences of known proteins in the species from which the sample was obtained or also include related species (e.g. homologs). In some cases, if the species of the sample is unknown then a database of some or all protein sequences may be used. The database may also contain the characteristics and/or sequences of any known protein variants and mutant proteins thereof.

In some embodiments, the software may comprise one or more algorithms, such as a machine learning, deep learning, statistical learning, supervised learning, unsupervised learning, clustering, expectation maximization, maximum likelihood estimation, Bayesian inference, linear regression, logistic regression, binary classification, multinomial classification, or other pattern recognition algorithm. For example, the software may perform the one or more algorithms to analyze the information regarding (i) the binding characteristic of each binding agent used, (ii) information from the database of proteins, and/or (iii) a list of locations observed (including in different cycles), in order to generate or assign a probable identity to each signal detected and/or a confidence (e.g., confidence level and/or confidence interval) for that information.

B. Use of Tags

In some further embodiments, the methods provided herein may include the use of tags that comprise any information characterizing a molecule. For example, the sample comprising one or more proteins, polypeptides, or peptides can be provided with a tag, e.g., nucleic acid tag, a DNA tag, or a recording tag. In some embodiments, the sample is provided with a plurality of recording tags. The recording tags may be associated or attached, directly or indirectly to the polypeptides. In some embodiments, the recording tags are attached to the polypeptides using any suitable means. In some aspects, the recording tag may be any suitable sequenceable moiety to which information can be transferred. In a particular embodiment, a single recording tag is attached to a polypeptide, preferably via the attachment to a N- or C-terminal amino acid. In another embodiment, multiple recording tags are attached to the polypeptide, such as to the lysine residues or peptide backbone. In some embodiments, a polypeptide labeled with multiple recording tags is fragmented or digested into smaller peptides, with each peptide labeled on average with one recording tag. The optional DNA tag or recording tag may provide information by containing a sample barcode, a fraction barcode, spatial barcode, and/or a compartment tag.

In some examples, the sample comprising one or more proteins, polypeptides, or peptides can be provided with a DNA tag, e.g., a recording tag. In some embodiments, the sample is provided with a plurality of recording tags. The recording tags may be associated or attached, directly or indirectly to the polypeptides. In some embodiments, the recording tags are attached to the polypeptides using any suitable means. In some aspects, the recording tag may be any suitable sequenceable moiety to which information can be transferred. In a particular embodiment, a single recording tag is attached to a polypeptide, preferably via the attachment to a N- or C-terminal amino acid. In another embodiment, multiple recording tags are attached to the polypeptide, such as to the lysine residues or peptide backbone. In some embodiments, a polypeptide labeled with multiple recording tags is fragmented or digested into smaller peptides, with each peptide labeled on average with one recording tag. The optional DNA tag or recording tag may provide information by containing a sample barcode, a fraction barcode, spatial barcode, and/or a compartment tag. In some embodiments, the DNA tags or recording tags comprise a sample barcode useful for sample multiplexing.

The recording tag may refer to a moiety, e.g., a chemical coupling moiety, a nucleic acid molecule, or a sequenceable polymer molecule (see, e.g., Niu et al., 2013, Nat. Chem. 5:282-292; Roy et al., 2015, Nat. Commun. 6:7237; Lutz, 2015, Macromolecules 48:4759-4767; each of which are incorporated by reference in its entirety) to which identifying information can be transferred. Identifying information can comprise any information characterizing a molecule such as information pertaining to sample, fraction, partition, source, etc. Additionally, the presence of UMI information can also be classified as identifying information. A recording tag may be directly linked to a polypeptide, linked to a polypeptide via a multifunctional linker, or associated with a polypeptide by virtue of its proximity (or co-localization) on a support. A recording tag may be linked via its 5′ end or 3′ end or at an internal site. A recording tag may further comprise other functional components, e.g., a universal priming site, unique molecular identifier, a barcode (e.g., a sample barcode, a fraction barcode, spatial barcode, a compartment tag, etc.), a spacer sequence that is complementary to a spacer sequence of another DNA tag, or any combination thereof.

A recording tag may comprise DNA, RNA, or polynucleotide analogs including PNA, γPNA, GNA, BNA, XNA, TNA, other polynucleotide analogs, or a combination thereof. A recording tag may be single stranded, or partially or completely double stranded. A recording tag may have a blunt end or overhanging end. In certain embodiments, all or a substantial amount of the macromolecules (e.g., at least 50%, 55%, 60%, 65%, 70%, 75%, 80%, 85%, 90%, 95%, 96%, 97%, 98%, 99%, or 100%) within a sample are labeled with a recording tag. In other embodiments, a subset of polypeptides within a sample are labeled with recording tags. In a particular embodiment, a subset of polypeptides from a sample undergo targeted (analyte specific) labeling with recording tags. For example, targeted recording tag labeling of proteins may be achieved using target protein-specific binding agents (e.g., antibodies, aptamers, etc.). In some embodiments, the recording tags are attached to polypeptides in a spatial sample in situ. In some embodiments, the recording tags are attached to the polypeptides prior to providing the sample on a support and/or prior to providing the polypeptides with a first detection agent. In some embodiments, the recording tags are attached to the polypeptides after providing the sample on the support and/or after providing the polypeptides with a first detection agent.

In some embodiments, the recording tag can include a sample identifying barcode. A sample barcode is useful in the multiplexed analysis of a set of samples in a single reaction vessel or immobilized to a single solid substrate or collection of solid substrates (e.g., a planar slide, population of beads contained in a single tube or vessel, etc.).

In certain embodiments, a DNA tag comprises an optional, unique molecular identifier (UMI), which provides a unique identifier tag for each macromolecules (e.g., polypeptide) to which the UMI is associated with. A UMI can be about 3 to about 40 bases, about 3 to about 30 bases, about 3 to about 20 bases, or about 3 to about 10 bases, or about 3 to about 8 bases. In some embodiments, a UMI is about 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 25 bases, 30 bases, 35 bases, or 40 bases in length. In certain embodiments, a recording tag comprises a universal priming site, e.g., a forward or 5′ universal priming site. A universal priming site is a nucleic acid sequence that may be used for priming a library amplification reaction and/or for sequencing. A universal priming site may include, but is not limited to, a priming site for PCR amplification, flow cell adaptor sequences that anneal to complementary oligonucleotides on flow cell surfaces (e.g., Illumina next generation sequencing), a sequencing priming site, or a combination thereof. A universal priming site can be about 10 bases to about 60 bases.

In any of the preceding embodiments, the transfer of identifying information (e.g., sample barcode) can be accomplished by ligation (e.g., an enzymatic or chemical ligation, a splint ligation, a sticky end ligation, a single-strand (ss) ligation such as a ssDNA ligation, or any combination thereof), a polymerase-mediated reaction (e.g., primer extension of single-stranded nucleic acid or double-stranded nucleic acid), or any combination thereof.

The recording tags may comprise a reactive moiety for a cognate reactive moiety present on the polypeptide (e.g., click chemistry labeling, photoaffinity labeling). Various types of linkages besides hybridization can be used to link the recording tag to a macromolecule. A suitable linker can be attached to various positions of the recording tag, such as the 3′ end, at an internal position, or within the linker attached to the 5′ end of the recording tag. The DNA tags or recording tags may further include other components including a unique molecular identifier, spacer, universal priming site, barcode, or combinations thereof. In some embodiments, the tag can be capped by addition of a universal reverse priming site via ligation, primer extension or other methods known in the art. In some embodiments, the DNA tag or recording tag comprises a universal forward priming site in the nucleic acid and a universal reverse priming site that is appended to the final extended nucleic acid.

In one embodiment, polypeptides with attached recording tags can be released from the sample after performing the method for analyzing the polypeptides as described in Section I. After release, the DNA or recording tag associated with the polypeptide may be used in or assessed by the techniques or procedures disclosed and/or claimed in U.S. Provisional Patent Application Nos. 62/330,841, 62/339,071, 62/376,886, 62/579,844, 62/582,312, 62/583,448, 62/579,870, 62/579,840, and 62/582,916, and International Patent Publication Nos. WO 2017/192633, and WO/2019/089836, and WO 2019/089851, which are incorporated herein by reference.

DNA tags, e.g. nucleic acid tag or recording tags, can be processed and analysed using a variety of nucleic acid sequencing methods. In some embodiments, the collection of tags can be concatenated. In some embodiments, the tags can be amplified prior to determining the sequence or being analyzed. Any combination of fractionation, enrichment, and subtraction methods, of the polypeptides before attachment to the solid support and/or of the resulting nucleic acid library can economize sequencing reads and improve measurement of low abundance species. Examples of sequencing methods include, but are not limited to, chain termination sequencing (Sanger sequencing); next generation sequencing methods, such as sequencing by synthesis, sequencing by ligation, sequencing by hybridization, polony sequencing, ion semiconductor sequencing, and pyrosequencing; and third generation sequencing methods, such as single molecule real time sequencing, nanopore-based sequencing, duplex interrupted sequencing, and direct imaging of DNA using advanced microscopy.

Suitable sequencing methods for use in the invention include, but are not limited to, sequencing by hybridization, sequencing by synthesis technology (e.g., HiSeg™ and Solexa™, Illumina), SMRT™ (Single Molecule Real Time) technology (Pacific Biosciences), true single molecule sequencing (e.g., HeliScope™, Helicos Biosciences), massively parallel next generation sequencing (e.g., SOLiD™, Applied Biosciences; Solexa and HiSeg™ Illumina), massively parallel semiconductor sequencing (e.g., Ion Torrent), pyrosequencing technology (e.g., GS FLX and GS Junior Systems, Roche/454), nanopore sequence (e.g., Oxford Nanopore Technologies).

In some embodiments, the analysis of the DNA tags is performed using a method compatible with the detection method for sensing the signal generated by the detectable label formed when the first and/or second detection agents are brought into sufficient proximity. In some embodiments, the analysis of the DNA tags is performed using the same device or platform for sensing the signal generated by the detectable label. In some embodiments, detection of the signal generated by the detectable label is compatible with assessment of the DNA tags. In some cases, the signal generated by the detectable label is the same type of signal used to analyze or assess the DNA tags.

In some particular embodiments, a photon detection device and sensing method, such as used by the 454 Life Sciences instrument, is suitable for detecting the signal generated by the detectable label and can be used to analyze the DNA tags. In some embodiments, both the signal from the detectable label and for assessing the DNA tags is a fluorescence based signal. In some embodiments, the platform for assessment of the DNA tags can be switched and used, or is compatible with chemistry treatments for the analysis methods provided herein, including to remove the NTAA of the polypeptides. In some cases, the platform for assessment of the DNA tags can be used for detection of the signal generated by the detectable label. In some embodiments, the methods provided herein for the polypeptide analysis using detection agents is compatible with nucleic acid-related methods.

In some embodiments, any additional information regarding the polypeptide contained in the DNA tag/recording tag may be correlated with the information from the polypeptide analysis using the binding agent(s). In some embodiments, the provided methods allow determination of at least a portion of the sequence of the polypeptide and the information regarding the polypeptide such as sample source.

### III. KITS AND ARTICLES OF MANUFACTURE

Provided herein are kits and articles of manufacture comprising components for polypeptide analysis using detection agents. In some embodiments, the kits further contain other reagents for treating and analyzing proteins, polypeptides, or peptides. The kits and articles of manufacture may include any one or more of the reagents and components used in the methods described in Section I and II. In some embodiments, the kit comprises a plurality of binding agents wherein each binding agent is associated with a second detection agent. In some aspects, the kits contain components for providing a polypeptide and an associated first detection agent joined to a support; contacting the polypeptide with a binding agent capable of binding to the polypeptide, wherein the binding agent is associated with a second detection agent, whereby binding between the polypeptide and the binding agent brings the first detection agent and the second detection agent into sufficient proximity to generate a detectable label; and detecting a signal generated by the detectable label. In some embodiments, the kits optionally include instructions for polypeptide analysis.

In some embodiments, the kits comprise one or more of the following components: binding agent(s) with associated second detection agent(s), first detection agent(s), linker(s) for immobilizing the polypeptide(s) and/or first detection agent(s), support(s), reagent(s) for attaching or joining the polypeptide and/or first detection agent, to each other or the support, and/or any reagents as described in the methods for analyzing proteins, polypeptides, or peptides, enzyme(s), buffer(s), etc. In some embodiments, the kits also include other components for treating the proteins, polypeptides, or peptides and analysis of the same. In one aspect, provided herein are components used to prepare a reaction mixture comprising two or more of the components described. In preferred embodiments, the reaction mixture is a solution. In preferred embodiments, the reaction mixture includes two or more of the following: binding agent(s) with associated second detection agent(s), first detection agent(s), linker(s) for immobilizing the polypeptide(s) and/or first detection agent(s), support(s), reagent(s) for attaching or joining the polypeptide and/or first detection agent, buffer(s), activating or blocking molecules, and/or any optional DNA tags or barcodes (e.g., recording tags).

In another aspect, disclosed herein is a kit comprising one or more binding agents, wherein at least some of the binding agents are each associated with a second detection agent. In some examples, the binding moiety of the binding agent is capable of binding to one or more N-terminal, internal, or C-terminal amino acids of the target peptide, or capable of binding to the one or more N-terminal, internal, or C-terminal amino acids of a peptide modified by a functionalizing reagent. The binding agents may be provided as a library of binding agents. The binding agents may be combined or provided in separate containers containing individual or subsets of the binding agents. In some embodiments, the kit further includes any molecules or components for activation of the first and/or second detection agents to generate a signal.

In some embodiments, the kits and articles of manufacture further comprise a plurality of nucleic acid molecules or oligonucleotides. In some embodiments, the kits include a plurality of barcodes. The barcode(s) may include a compartment barcode, a partition barcode, a sample barcode, a fraction barcode, or any combination thereof. In some cases, the barcode comprises a unique molecule identifier (UMI). In some examples, the barcode comprises a DNA molecule, DNA with pseudo-complementary bases, an RNA molecule, a BNA molecule, an XNA molecule, a LNA molecule, a PNA molecule, a γPNA molecule, a non-nucleic acid sequenceable polymer, e.g., a polysaccharide, a polypeptide, a peptide, or a polyamide, or a combination thereof. In some embodiments, the barcodes are configured to attach the target macromolecules, e.g., the proteins, in the sample or to attach to nucleic components associated with the targets.

In some embodiments, the kit further comprises reagents for treating the proteins or polypeptides. Any combination of fractionation, enrichment, and subtraction methods, of the proteins may be performed. For example, the reagent may be used to fragment or digest the proteins. In some cases, the kit comprises reagents and components to fractionate, isolate, subtract, enrich proteins. In some examples, the kits further comprises a protease such as trypsin, LysN, or LysC. In some embodiments, the kit comprises a support for immobilizing the one or more or polypeptides and reagents for immobilizing the or polypeptides on a support.

In some embodiments, the kit also comprises one or more buffers or reaction fluids necessary for any of the binding reaction to occur. Buffers including wash buffers, reaction buffers, and binding buffers, elution buffers and the like are known to those or ordinary skill in the arts. In some embodiments, the kits further include buffers and other components to accompany other reagents described herein. The reagents, buffers, and other components may be provided in vials (such as sealed vials), vessels, ampules, bottles, jars, flexible packaging (e.g., sealed Mylar or plastic bags), and the like. Any of the components of the kits may be sterilized and/or sealed.

In some embodiments, the kit further includes one or more reagents for nucleic acid sequence analysis. In some examples, the reagent for sequence analysis is for use in sequencing by synthesis, sequencing by ligation, single molecule sequencing, single molecule fluorescent sequencing, sequencing by hybridization, polony sequencing, ion semiconductor sequencing, pyrosequencing, single molecule real-time sequencing, nanopore-based sequencing, or direct imaging of DNA using advanced microscopy, or any combination thereof.

In addition to above-mentioned components, the subject kits may further include instructions for using the components of the kit to practice the subject methods, i.e., instructions for sample preparation, treatment and/or analysis. The kits described herein may also include other materials desirable from a commercial and user standpoint, including other buffers, diluents, filters, syringes, and package inserts with instructions for performing any methods described herein.

Any of the above-mentioned kit components, and any molecule, molecular complex or conjugate, reagent (e.g., chemical or biological reagents), agent, structure (e.g., support, surface, particle, or bead), reaction intermediate, reaction product, binding complex, or any other article of manufacture disclosed and/or used in the exemplary kits and methods, may be provided separately or in any suitable combination in order to form a kit.

### IV. EXEMPLARY EMBODIMENTS

Among the provided embodiments are:

1. A method for analyzing a polypeptide, comprising the steps of:

(a) providing a polypeptide and an associated first detection agent joined to a support;

(b) contacting the polypeptide with a binding agent capable of binding to the polypeptide, wherein the binding agent is associated with a second detection agent, whereby binding between the polypeptide and the binding agent brings the first detection agent and the second detection agent into sufficient proximity to generate a detectable label;

(c) detecting a signal generated by the detectable label; and repeating step (b) and step (c) sequentially one or more times.

2. The method of embodiment 1, wherein the first detection agent and the second detection agent, when brought into sufficient proximity, forms a detectable label.

3. The method of embodiment 1, wherein the first detection agent and the second detection agent, when brought into sufficient proximity, forms a detectable label precursor, and further comprising activating the detectable label precursor to form a detectable label.

4. The method of embodiment 3, wherein activating the detectable label precursor comprises binding an activating agent to a complex of the first detection agent and the second detection agent.

5. The method of embodiment 4, wherein the activating agent is an allosteric activator of the first and/or second detection agent.

6. The method of embodiment 1, wherein generating the detectable label in step (b) comprises removing inhibition of the first and/or second detection agent.

7. The method of embodiment 1, wherein generating the detectable label in step (b) comprises the second detection agent displacing a repressor protein or a blocking molecule from the first detection agent.

8. The method of embodiment 1, wherein generating the detectable label in step (b) comprises the second detection agent cleaving a repressor protein or a blocking molecule bound to the first detection agent.

9. The method of any one of embodiments 1-8, wherein the detectable label is selected from a bioluminescent label, a chemiluminescent label, a chromophore label, an enzymatic label, and a fluorescent label.

10. The method of any one of embodiments 1-9, wherein the method is performed on a plurality of polypeptides.

11. The method of embodiment 10, further comprising providing the plurality of polypeptides with a first detection agent during or prior to step (a).

12. The method of embodiment 11, wherein the polypeptides are immobilized to the support prior to providing the polypeptides with the first detection agent.

13. The method of embodiment 11, wherein the polypeptides are immobilized to the support after providing the polypeptides with the first detection agent.

14. The method of any one of embodiments 1-13, wherein the first and second detection agents are individually inactive.

15. The method of any one of embodiments 1-14, wherein the first detection agent is a nucleic acid, a protein, a peptide, an antibody, an aptamer, a small-molecule compound, or a portion thereof.

16. The method of embodiment 15, wherein the first detection agent is an enzyme.

17. The method of embodiment 15, wherein the first detection agent is a first subunit of a split enzyme.

18. The method of embodiment 15, wherein the first detection agent is an affinity molecule.

19. The method of embodiment 15, wherein the first detection agent is a first subunit of a split affinity molecule.

20. The method of embodiment 15, wherein the first detection agent is a fluorophore or chromophore, or a portion thereof.

21. The method of embodiment 15, wherein the first detection agent comprises a repressor protein or blocking molecule.

22. The method of embodiment 15, wherein the first detection agent comprises an inducer protein.

23. The method of any one of embodiments 1-22,wherein the second detection agent is a nucleic acid, a protein, a peptide, an antibody, an aptamer, a small-molecule compound, or a portion thereof.

24. The method of embodiment 23, wherein the second detection agent is an enzyme.

25. The method of embodiment 23, wherein the second detection agent is a second subunit of a split enzyme.

26. The method of embodiment 23, wherein the second detection agent is an affinity molecule.

27. The method of embodiment 23, wherein the second detection agent is a second subunit of a split affinity molecule.

28. The method of embodiment 23, wherein the second detection agent is a fluorophore or chromophore, or a portion thereof.

29. The method of embodiment 23, wherein the second detection agent comprises a repressor protein or blocking molecule.

30. The method of embodiment 23, wherein the second detection agent comprises an inducer protein or an activating molecule.

31. The method of embodiment 20 or embodiment 28, wherein the fluorophore is green fluorescent protein enhanced green fluorescent protein.

32. The method of embodiment 15 or embodiment 23, wherein the protein is yeast Gal4 or ubiquitin.

33. The method of any one of embodiments 16, 17, 24, and 25, wherein the enzyme is carbonic anhydrase, T7 RNA polymerase, beta-galactosidase, dihydrofolate reductase, beta-lactamase, tobacco etch virus protease, luciferase, or horseradish peroxidase.

34. The method of any one of embodiments 1-33, wherein the first and second detection agents comprise separate portions of a FRET system or a BRET system.

35. The method of any one of embodiments 1-34, wherein the first and/or second detection agents generate a detectable signal upon introduction to light.

36. The method of any one of embodiments 1-34, wherein the first and/or second detection agents generate a detectable signal upon introduction to an activating agent.

37. The method of embodiment 4 or embodiment 36, wherein the activating agent comprises a chemical reagent, a non-biological reagent, a biological reagent, or a combination thereof.

38. The method of embodiment 37, wherein the activating agent comprises a polypeptide or a protein.

39. The method of embodiment 37, wherein the activating agent comprises a metal ion.

40. The method of any one of embodiments 1-39, wherein the signal is generated by the second detection agent in the presence of the first detection agent.

41. The method of any one of embodiments 1-39, wherein the signal is generated by the first detection agent in the presence of the second detection agent.

42. The method of any one of embodiments 1-39, wherein the signal is generated by the first detection agent upon joining to or contacting with the second detection agent.

43. The method of any one of embodiments 1-42, wherein the signal generated by the first and/or second detection agents is luminescent-based or fluorescent-based.

44. The method of any one of embodiments 1-43, wherein the first detection agent is directly or indirectly joined to the polypeptide.

45. The method of any one of embodiments 1-44, wherein the first detection agent is in proximity to the polypeptide.

46. The method of any one of embodiments 1-45, wherein the second detection agent is directly or indirectly joined to the binding agent.

47. The method of any one of embodiments 1-46, wherein the first detection agent is associated to the polypeptide via a linker.

48. The method of embodiment 47, wherein the linker comprises:

a moiety for associating with the polypeptide; and

a moiety for associating with the first detection agent.

49. The method of embodiment 47 or embodiment 48, wherein the linker comprises a biotin.

50. The method of embodiment 49, wherein the first detection agent is configured to bind to the biotin.

51. The method of embodiment 49 or embodiment 50, wherein the first detection agent is associated with a hapten-binding group.

52. The method of embodiment 51, wherein the hapten-binding group is streptavidin.

53. The method of embodiment 51 or embodiment 52, wherein the hapten-binding group and the first detection agent are chemically or genetically attached.

54. The method of embodiment 53, wherein the chemical attachment is a covalent attachment via a linker molecule.

55. The method of any one of embodiments 47-54, wherein the linker is a tri-functional linker.

56. The method of embodiment 55, wherein the tri-functional linker comprises:

a moiety to associating with the polypeptide;

a moiety for associating with the support; and

a moiety for associating with the first detection agent.

57. The method of embodiment 55 and embodiment 56, wherein the tri-functional linker has the following structure:

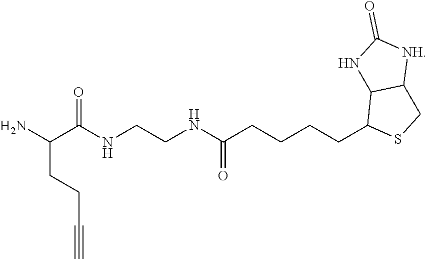 [SMILES: C#CCCC(N)C(=O)NCCNC(=O)CCCCC1SCC2NC(=O)NC21]

58. The method of embodiment 55 and embodiment 56, wherein the tri-functional linker has the following structure:

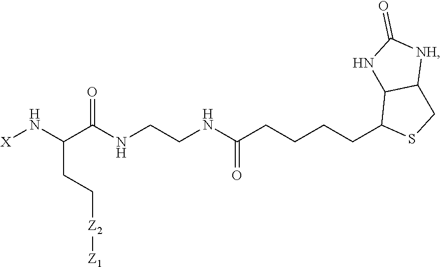

wherein:

X is the peptide; and

Z₁-Z₂ is C≡C and is capable of binding to the support.

59. The method of any one of embodiments 1-58, wherein the detection in step (c) employs a field effect transistor (FET) sensor.

60. The method of any one of embodiments 1-58, wherein the detection in step (c) employs chemical detection or optical detection.

61. The method of any one of embodiments 1-58, wherein the detection in step (c) is a detection of a change in pH.

62. The method of embodiment 61, wherein the change in pH is the result of a release of protons (H+).

63. The method of any one of embodiments 1-60, wherein the detection in step (c) is a detection of photons.

64. The method of any one of embodiments 1-60, wherein the detection in step (c) is a detection of fluorescence.

65. The method of any one of embodiments 1-64, wherein the signal generated by the first and/or second detection agents is quenched or deactivated after step (c) and/or prior to repeating step (b).

66. The method of any one of embodiments 1-65, wherein the second detection agent is released from the first detection agent after step (c) and/or prior to repeating step (b).

67. The method of any one of embodiments 1-65, wherein the binding agent is released from the polypeptide after step (c) and/or prior to repeating step (b).

68. The method of any one of embodiments 1-67, further comprising:

(d) removing a portion of the polypeptide.

69. The method of embodiment 68, wherein step (d) is performed after step (c) and before repeating step (b).

70. The method of embodiment 69, wherein steps (b)-(d) are repeated sequentially one or more times.

71. The method of any one of embodiments 68-70, wherein the portion of the polypeptide is removed with a bound binding agent.

72. The method of any one of embodiments 68-71, wherein step (d) is performed by contacting the polypeptide with a chemical or enzymatic reagent.

73. The method of any one of embodiments 68-72, wherein step (d) dissociates the first detection agent from the polypeptide.

74. The method of any one of embodiments 68-73, wherein the portion of the polypeptide removed comprises the N-terminal amino acid, thereby yielding a newly exposed NTAA of the polypeptide.

75. The method of any one of embodiments 72-74, wherein the chemical or enzymatic reagent selectively removes an N-terminal amino acid (NTAA) of the polypeptide.

76. The method of any one of embodiments 68-75, wherein one amino acid is removed from the polypeptide.

77. The method of any one of embodiments 68-75, wherein two amino acids are removed from the polypeptide.

78. The method of any one of embodiments 72-77, wherein the enzymatic reagent comprises a carboxypeptidase or an aminopeptidase or a variant, mutant, or modified protein thereof; a hydrolase or a variant, mutant, or modified protein thereof, a modified amino acid tRNA synthetase, an Edmanase enzyme, or any combination thereof.

79. The method of any one of embodiments 74-78, wherein the amino acid is removed from the polypeptide by mild Edman degradation or treatment with anhydrous TFA.

80. The method of any one of embodiments 68-79, wherein the removed portion of the polypeptide comprises a modified amino acid residue of the polypeptide.

81. The method of any one of embodiments 1-80, further comprising treating the polypeptide with a reagent for modifying a terminal amino acid of the polypeptide.

82. The method of embodiment 81, wherein the reagent for modifying a terminal amino acid of a polypeptide comprises a chemical reagent or an enzymatic agent.

83. The method of embodiment 82, wherein polypeptide is contacted with the reagent for modifying a terminal amino acid prior to step (d).

84. The method of embodiment 82, wherein the polypeptide is contacted with the reagent for modifying a terminal amino acid prior to step (b).

85. The method of embodiment 82, wherein the polypeptide is contacted with the reagent for modifying a terminal amino acid after step (c).

86. The method of any one of embodiments 68-85, further comprising providing the polypeptide with the first detection agent after step (d) and prior to repeating step (b).

87. The method of any one of embodiments 82-86, wherein the chemical reagent is selected from the group consisting of a phenyl isothiocyanate (PITC), a nitro-PITC, a sulfo-PITC, a phenyl isocyanate (PIC), a nitro-PIC, a sulfo-PIC, Cbz-Cl (benzyl chloroformate) or Cbz-OSu (benzyloxycarbonyl N-succinimide), an anhydride, a 1-fluoro-2,4-dinitrobenzene (Sanger's reagent, DNFB), dansyl chloride (DNS-Cl, or 1-dimethylaminonaphthalene-5-sulfonyl chloride), 4-sulfonyl-2-nitrofluorobenzene (SNFB), 2-Pyridinecarboxaldehyde, 2-Formylphenylboronic acid, 2-Acetylphenylboronic acid, 1-Fluoro-2,4-dinitrobenzene, 4-Chloro-7-nitrobenzofurazan, Pentafluorophenylisothiocyanate, 4-(Trifluoromethoxy)-phenylisothiocyanate, 4-(Trifluoromethyl)-phenylisothiocyanate, 3-(Carboxylic acid)-phenylisothiocyanate, 3-(Trifluoromethyl)-phenylisothiocyanate, 1-Naphthylisothiocyanate, N-nitroimidazole-1-carboximidamide, N,N′-Bis(pivaloyl)-1H-pyrazole-1-carboxamidine, N,N′-Bis(benzyloxycarbonyl)-1H-pyrazole-1-carboxamidine, an acetylating reagent, a guanidinylation reagent, a thioacylation reagent, a thioacetylation reagent, a thiobenzylation reagent, and a diheterocyclic methanimine reagent, or a derivative thereof.

88. The method of any one of embodiments 1-87, wherein the binding agent binds to a single amino acid residue, a dipeptide, a tripeptide or a post-translational modification of the polypeptide.

89. The method of any one of embodiments 1-88, wherein the binding agent binds to an N-terminal amino acid residue, a C-terminal amino acid residue, or an internal amino acid residue.

90. The method of any one of embodiments 1-88, wherein the binding agent binds to an N-terminal peptide, a C-terminal peptide, or an internal peptide.

91. The method of embodiment 89, wherein the binding agent is configured to bind to a C-terminal amino acid residue of the polypeptide.

92. The method of embodiment 89, wherein the binding agent is configured to bind to an N-terminal amino acid residue of the polypeptide.

93. The method of any one of embodiments 1-92, wherein the binding agent is a polypeptide or protein.

94. The method of embodiment 93, wherein the binding agent is an aminopeptidase or variant, mutant, or modified protein thereof an aminoacyl tRNA synthetase or variant, mutant, or modified protein thereof an anticalin or variant, mutant, or modified protein thereof a ClpS, ClpS2, or variant, mutant, or modified protein thereof; a UBR box protein or variant, mutant, or modified protein thereof; or a modified small molecule that binds amino acid(s), i.e. vancomycin or a variant, mutant, or modified molecule thereof; or an antibody or binding fragment thereof; or any combination thereof.

95. The method of any one of embodiments 1-89, wherein the binding agent and the second detection agent are joined by a linker.

96. The method of any one of embodiments 1-95, wherein step (b) comprises contacting the polypeptide with a plurality of binding agents as a mixture, and each binding agent is associated with a second detection agent.

97. The method of embodiment 96, wherein each binding agent is associated with a different second detection agent.

98. The method of embodiment 97, wherein the signal generated by the first and/or second detection agent is different for each binding agent.

99. The method of embodiment 98, wherein each of the second detection agents of the plurality of binding agents, when in sufficient proximity with the first detection agent, generates a detectable label dependent on the identity of the target of the binding agent, to which each of the plurality of binding agents selectively bind.

100. The method of any one of embodiments 1-99, wherein each cycle of the method comprises in step (b), providing one type of binding agent to the polypeptides.

101. The method of any one of embodiments 1-100, wherein the polypeptide is indirectly joined to a support.

102. The method of any one of embodiments 1-101, wherein the support is a planar substrate.

103. The method of any one of embodiments 1-101, wherein the support is a bead, a microbead, an array, a glass surface, a silicon surface, a plastic surface, a filter, a membrane, nylon, a silicon wafer chip, a flow through chip, a biochip including signal transducing electronics, a microtitre well, an ELISA plate, a spinning interferometry disc, a nitrocellulose membrane, a nitrocellulose-based polymer surface, a nanoparticle, or a microsphere.

104. The method of any one of embodiments 1-101, wherein the support comprises a three-dimensional support (e.g., a porous matrix or a bead).

105. The method of any one of embodiments 1-104, wherein the support comprises a reacting agent.

106. The method of any one of embodiment 105, wherein the reacting agent comprises an azide group.

107. The method of any one of embodiments 106, wherein the polypeptide is linked to the support by reaction of an alkyline group in the trifunctional linker and an azide group present on the support.

108. The method of any one of embodiments 1-107, wherein the polypeptide is obtained by fragmenting protein(s) from a biological sample.

109. The method of embodiment 108, wherein the fragmenting is performed by contacting the protein(s) with a protease.

110. The method of any one of embodiments 1-109, wherein method is performed on a plurality of polypeptides of unknown identity isolated from a sample.

111. A kit comprising:

a support;

a first detection agent configured to be associated with a polypeptide, directly or indirectly, joined to a support;

a binding agent capable of binding to the polypeptide, wherein the binding agent is associated with a second detection agent, wherein binding between the polypeptide and the binding agent brings the first detection agent and the second detection agent into sufficient proximity to generate a detectable label; and

a reagent for modifying a terminal amino acid of the polypeptide and/or a reagent for removing a portion of the polypeptide.

112. The kit of embodiment 111, wherein the kit comprises a plurality of the binding agents.

113. The kit of embodiment 111 or embodiment 112, wherein the first detection agent is a nucleic acid, a peptide, a protein, an antibody, an aptamer or a small-molecule compound.

114. The kit of embodiment 111, wherein first detection agent comprises:

an enzyme;

a first subunit of a split enzyme;

an affinity molecule;

a first subunit of a split affinity molecule;

a fluorophore or chromophore, or a portion thereof;

a repressor protein or blocking molecule; or

an inducer protein.

115. The kit of any one of embodiments 109-112,wherein the second detection agent is a nucleic acid, a peptide, a protein, an antibody, an aptamer or a small-molecule compound.

116. The kit of embodiment 113, wherein the second detection agent comprises:

an enzyme;

a second subunit of a split enzyme;

an affinity molecule;

a second subunit of a split affinity molecule;

a fluorophore or chromophore, or a portion thereof;

a repressor protein or blocking molecule; or

an inducer protein or an activator molecule.

117. The kit of embodiment 114 or embodiment 116, wherein the enzyme is carbonic anhydrase, T7 RNA polymerase, beta-galactosidase, dihydrofolate reductase, beta-lactamase, tobacco etch virus protease, luciferase, or horseradish peroxidase.

118. The kit of embodiment 114 or embodiment 116, wherein the fluorophore is green fluorescent protein enhanced green fluorescent protein.

119. The kit of embodiment 113 or embodiment 115, wherein the protein is yeast Gal4 or ubiquitin.

120. The kit of any one of embodiments 111-119, wherein the first and second detection agents comprise separate portions of a FRET system or a BRET system.

121. The kit of any one of embodiments 111-120, wherein the first detection agent and the second detection agent, when brought into sufficient proximity, forms a detectable label.

122. The kit of any one of embodiments 111-120, wherein the first detection agent and the second detection agent, when brought into sufficient proximity and activated, forms a detectable label precursor.

123. The kit of embodiment 122, further comprising an activating agent for activation of the detectable label precursor which binds to a complex of the first detection agent and the second detection agent.

124. The kit of embodiment 123, wherein the activating agent is an allosteric activator of the first and/or second detection agent.

125. The kit of embodiment 111-120, wherein the detectable label is generated upon removal of inhibition of the first and/or second detection agent.

126. The kit of embodiment 111-120, wherein the detectable label is generated upon the second detection agent displacing a repressor protein or a blocking molecule from the first detection agent.

127. The kit of embodiment 111-120, wherein the detectable label is generated upon the second detection agent cleaving a repressor protein or a blocking molecule bound to the first detection agent.

128. The kit of embodiment 111-127, wherein the detectable label is selected from a bioluminescent label, a chemiluminescent label, a chromophore label, an enzymatic label, and a fluorescent label.

129. The kit of any one of embodiments 111-128, wherein the first and/or second detection agents generate a detectable signal upon introduction to light.

130. The kit of any one of embodiments 111-128, wherein the first and/or second detection agents generate a detectable signal upon introduction to an activating agent.

131. The kit of embodiment 123 or embodiment 130, wherein the activating agent comprises a chemical reagent, a non-biological reagent, a biological reagent, or a combination thereof.

132. The kit of embodiment 131, wherein the activating agent comprises a polypeptide or a protein.

133. The kit of embodiment 131, wherein the activating agent comprises a metal ion.

134. The kit of any one of embodiments 111-133, further comprising a linker for associating the first detection agent to the polypeptide.

135. The kit of embodiment 134, wherein the linker comprises:

a moiety for associating with the polypeptide; and

a moiety for associating with the first detection agent.

136. The kit of embodiment 134 or embodiment 135, wherein the linker comprises a biotin.

137. The kit of embodiment 136, wherein the first detection agent is configured to bind to the biotin.

138. The kit of embodiment 136 or embodiment 137, wherein the first detection agent is associated with a hapten-binding group.

139. The kit of embodiment 138, wherein the hapten-binding group is streptavidin.

140. The kit of any one of embodiments 134-139, wherein the linker is a tri-functional linker.

141. The kit of embodiment 140, wherein the tri-functional linker comprises:

a moiety to associating with the polypeptide;

a moiety for associating with the support; and

a moiety for associating with the first detection agent.

142. The kit of embodiment 140 and embodiment 141, wherein the tri-functional linker has the following structure:

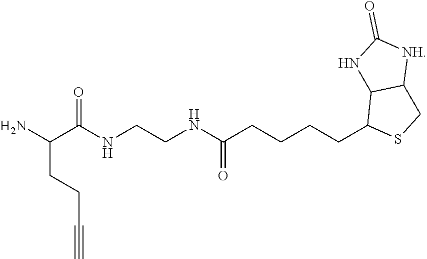 [SMILES: C#CCCC(N)C(=O)NCCNC(=O)CCCCC1SCC2NC(=O)NC21]

143. The kit of embodiment 140 and embodiment 141, wherein the tri-functional linker has the following structure:

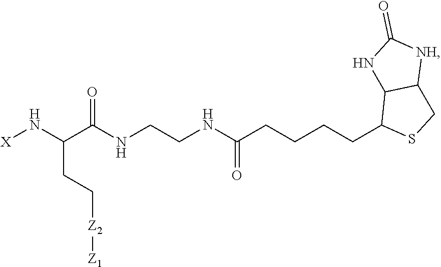

wherein:

X is the peptide; and

Z₁-Z₂ is C≡C and is capable of binding to the support.

144. The kit of any one of embodiments 111-143, wherein the reagent for modifying a terminal amino acid of a polypeptide comprises a chemical agent or an enzymatic agent.

145. The kit of any one of embodiments 111-144, wherein the reagent for removing a portion of the polypeptide comprises a chemical agent or an enzymatic agent.

146. The kit of any one of embodiments 111-145, wherein the binding agent binds to a single amino acid residue, a dipeptide, a tripeptide or a post-translational modification of the polypeptide.

147. A method for analyzing a polypeptide, comprising the steps of:

a. providing a polypeptide and an associated first detection agent attached to a solid support;

b. contacting the polypeptide with a binding agent capable of binding to the polypeptide, wherein the binding agent is joined to a second detection agent, whereby binding between the polypeptide and the binding agent brings the first detection agent and the second detection agent into sufficient proximity to interact with each other and generate a detectable label;

c. detecting a signal generated by the detectable label; and

d. repeating step (b) and step (c) sequentially one or more times.

148. The method of embodiment 147, wherein analyzing the polypeptide comprises identifying at least a portion of an amino acid sequence of the polypeptide.

149. The method of any one of embodiments 147-148, wherein the first detection agent and the second detection agent, when brought into sufficient proximity, forms a detectable label precursor, and further comprising activating the detectable label precursor to form a detectable label.

150. The method of embodiment 149, wherein activating the detectable label precursor comprises binding an activating agent to a complex of the first detection agent and the second detection agent, wherein the activating agent is an allosteric activator of the first and/or second detection agent.

151. The method of any one of embodiments 147-150, wherein generating the detectable label in step (b) comprises the second detection agent displacing a repressor protein or a blocking molecule from the first detection agent.

152. The method of any one of embodiments 147-150, wherein the detectable label is selected from the group consisting of a bioluminescent label, a chemiluminescent label, a chromophore label, an enzymatic label, and a fluorescent label.

153. The method of any one of embodiments 147-150, wherein the first detection agent is a first subunit of a split enzyme, the second detection agent is a second subunit of a split enzyme, and both the first detection agent and the second detection agent are enzymatically inactive.

154. The method of embodiment 153, wherein the first detection agent and the second detection agent comprise polypeptides.

155. The method of embodiment 153, wherein the first detection agent and the second detection agent comprise polynucleotides.

156. The method of embodiment 153, wherein the detectable label is an enzyme assembled from the first detection agent and the second detection agent interacting with each other, or a product of an enzymatic reaction catalyzed by the enzyme.

157. The method of embodiment 154, wherein the enzyme is a fluorescent protein.

158. The method of any one of embodiments 147-157, wherein the first detection agent is associated with the polypeptide via a linker, wherein the linker is a tri-functional linker that comprises:

a. a moiety to associating with the polypeptide;

b. a moiety for associating with the support; and

c. a moiety for associating with the first detection agent.

159. The method of any one of embodiments 147-158, wherein the first detection agent and the second detection agent do not comprise a polynucleotide, and do not undergo a polynucleotide-based hybridization or enzymatic covalent ligation to each other during generation of the detectable label.

160. The method of any one of embodiments 147-159, wherein the detection in step (c) employs:

(a) a field effect transistor (FET) sensor;

(b) a chemical detection means;

(c) an optical detection means; or

(d) a detection of a change in pH.

161. The method of any one of embodiments 147-160, wherein the detection in step (c) is a detection of fluorescence.

162. The method of any one of embodiments 147-161, wherein the first detection agent and the second detection agent, when brought into sufficient proximity, are interacting through non-covalent interactions to form the detectable label.

163. The method of any one of embodiments 147-162, wherein step (b) comprises contacting the polypeptide with a plurality of binding agents as a mixture; each binding agent is joined to a different second detection agent; and the signal generated by the detectable label is different for each binding agent.

164. The method of any one of embodiments 147-163, further comprising: (d) removing a portion of the polypeptide, wherein step (d) is performed after step (c) and before repeating step (b), and wherein steps (b)-(d) are repeated sequentially one or more times.

165. The method of embodiment 164, wherein step (b) comprises contacting the polypeptide with a plurality of binding agents as a mixture; each binding agent is joined to a different second detection agent; and the signal generated by the detectable label is different for each binding agent.

166. The method of embodiment 164, wherein in each repetition during step (b) the polypeptide is contacted with a different binding agent that is joined to the same second detection agent.

167. The method of embodiment 164, wherein the portion of the polypeptide removed comprises the N-terminal amino acid (NTAA), thereby yielding a newly exposed NTAA of the polypeptide.

168. A method of identifying one or more binding events between a plurality of binding agents and a plurality of polypeptides, comprising: (a) providing a plurality of polypeptides attached to a solid support, wherein each polypeptide from the plurality of polypeptides is associated with a first detection agent; (b) contacting a polypeptide from the plurality of polypeptides with a plurality of binding agents, wherein at least one binding agent from the plurality of binding agents is capable of binding to the polypeptide, and wherein each binding agent from the plurality of binding agents is joined to a second detection agent, whereby binding between the polypeptide and the at least one binding agent brings the first detection agent and the second detection agent into sufficient proximity to interact with each other and generate a detectable label; (c) detecting a signal generated by the detectable label, thereby identifying the binding between the polypeptide and the at least one binding agent; (d) optionally, removing a portion of the polypeptide; and repeating steps (b), (c) and (d) sequentially one or more times.

### V. EXAMPLES

The following examples are offered to illustrate but not to limit the methods, compositions, and uses provided herein. Certain aspects of the present invention, including, but not limited to, embodiments for the Proteocode™ polypeptide sequencing assay, methods for attachment of polypeptides or nucleotide-polypeptide conjugates to a solid support, methods of making nucleotide-polypeptide conjugates, methods of generating specific binding agents recognizing a terminal amino acid of a polypeptide immobilized on the solid support, reagents and methods for modifying and/or removing an N-terminal amino acid from an immobilized polypeptide were disclosed in earlier published application US 20190145982 A1, US 20200348308 A1, US 20200348307 A1, WO 2020/223000, the contents of which are incorporated herein by reference in its entirety.

### Example 1. Carbonic Anhydrase as Split Enzyme

Carbonic anhydrases form a family of enzymes that catalyze the rapid interconversion of carbon dioxide and water to bicarbonate and protons, a reversible reaction that occurs relatively slowly in the absence of a catalyst. The active site of most carbonic anhydrases contains a zinc ion; they are therefore classified as metalloenzymes. The reaction catalyzed by carbonic anhydrase (CA) is as follows:

CO₂+H₂O→H₂CO₃→H⁺+HCO₃.

With a kcat (turnover) of 10⁴-10⁶ per second, the reaction rate of carbonic anhydrase is one of the fastest of all enzymes, and its rate is typically limited by the diffusion rate of its substrates.

In the present example, a peptide and a first detection agent (first portion of a split CA) are joined to a solid support by way of linker L-1. The NTAA of the peptide is identified by sequential binding of up to twenty different binding agents (cognate and non-cognate), each selective for one of the twenty naturally-occurring amino acids. Each of these binding agents is associated with a second detection agent (second portion of a split CA), optionally via a linker. The first portion of the split CA is also joined by a linker to streptavidin, which is capable of binding to the biotin portion of linker L-1.

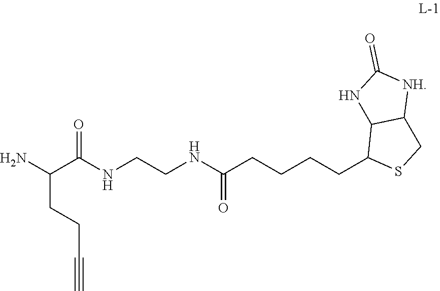 [SMILES: C#CCCC(N)C(=O)NCCNC(=O)CCCCC1SCC2NC(=O)NC21]

More specifically, the free amine of linker L-1 is used to form an amide bond with the peptide. The alkynyl group (triple bond) may then be used for attachment to a solid support bearing an azide group by way of click chemistry (while the solid support is not shown, is should be understood that the free alkynyl group is intended to represent the point of attachment to the solid support). Together, the peptide and the first detection agent are joined to the solid support by joining the first detection agent (the first portion of split CA) to linker L-1 via biotin-streptavidin binding.

A cognate binding agent is used that is capable of selectively binding to the NTAA of peptide, wherein the cognate binding agent comprises second detection agent. In this example, the first and second portions of a split CA, when joined forms a detectable label (e.g., functional CA), results in the release of protons, as depicted by H⁺ generation. After the signal has been read, the cognate binding agent linked to the second detection agent may be removed from the peptide and the NTAA cleaved, which can be in the same or separate steps, thereby yielding a newly exposed NTAA. The steps noted above may then be repeated on the newly exposed NTAA. To the extent that the first detection agent is lost or depleted upon removal of the NTAA (e.g., by dissociation of the biotin-streptavidin interaction), it may be replace or replenished prior to repeating the cycle.

The method can be performed in a well using a silicon wafer. A pH change due to the release of protons may be used to detect the presence of a cognate binding agent selectively bound to the NTAA, and record its position on the two-dimensional surface. When the peptide is exposed to a non-cognate binding agent, or upon removal of the cognate binding agent, no signal is detected.

In a representative cycle of the method, in step 1, the peptide and an associated first detection agent are provided on a solid support, the peptide having an NTAA. In step 2, the peptide is contacted with a cognate binding agent capable of selectively binding to the NTAA of the peptide, wherein the cognate binding agent comprises a second detection agent. In step 3, the signal generated by the first and second detection agents associated with the selective binding of the NTAA by the cognate binding agent is read. In step 4, the NTAA is removed, such as by Edman degradation, thereby yielding a newly exposed NTAA. The cycle is then repeated with the newly exposed NTAA in place of the NTAA from the prior cycle.

### Example 2. T7 RNA Polymerase as Split Enzyme

In addition to carbonic anhydrase, as illustrated in Example 1, any proteins or enzymes that loses activity when split, but regains activity when co-localized, may be used in the methods disclosed herein. For example, nucleic acids with functional activity have also been split (e.g., DNAzymes and aptamers) and can be utilized in these methods. This example describes using T7 RNA polymerase as the split enzyme (e.g., first and second detection agent). This enzyme catalyzes synthesis of RNA in the 5′ to 3′ direction in the presence of a DNA template containing a T7 phage promoter.

The split version of T7 RNAP was originally discovered during purification and shown to be active in vitro. While the catalytic core and DNA-binding domain are both located on the C-terminal fragment of split T7 RNAP (sT7 RNAP), the N-terminal fragment is needed for transcript elongation. Specific variants of split T7 RNA polymerases were engineered and can be used in the claimed methods that assembled into a functional enzyme dependent on fused interaction partners (for example, the N-29-1, N-29-8 and the C-terminal RNAP variants disclosed in Pu J, et al., Evolution of a split RNA polymerase as a versatile biosensor platform. Nat Chem Biol. 2017 April; 13(4):432-438). A sT7 RNAP enzyme incorporating circularized homopolymer DNA with different RNA polymerase binding sites generates predictable charge signals that are quite similar to those resulting from nucleic acid sequence on Ion Platforms. The reaction catalyzed by T7 RNAP is as follows, with a kcat (turnover) rate of 200-300 per second:

NTP+RNA→RNA+1+PPi+

As described above in the context of split CA, joining of the split T7 RAP polymerase results in proton generation, which can be read as in indication of the cognate binding agent selectively binding to the NTAA of the peptide.

### Example 3. Fluorescent Proteins as Split Enzymes

Molecular engineering of fluorescent proteins, such as GFP, has produced several variants with altered spectral characteristics. Moreover, selected fragments of fluorescent proteins can associate with each other to produce functional bimolecular fluorescent complexes, allowing for use them as split fluorescent proteins having different excitation/emission characteristics. Such complementation provides an opportunity for detection of a binding reaction if the fluorescent protein fragments can associate only when they are brought together by interactions between an immobilized polypeptide and binding agents, both fused to fluorescent protein complementary fragments. Interestingly, different fluorescent protein variants can support heterologous fluorescent complex formation generating complexes with distinct spectral characteristics (detectable labels). For example, four fluorescent proteins (namely green, yellow, cyan and blue fluorescent proteins, or GFP, YFP, CFP and BFP, respectively) can be split to two non-fluorescent fragments and reassembled using heterologous fragments, producing fluorescent proteins with different spectral characteristics. In one particular example, the 155-238 amino acid (aa) fragment of CFP (CC155, SEQ ID NO: 1) can be produce functional fluorescent proteins with different spectral characteristics when brought together through fusions with interacting partners with the 1-172 aa fragment of GFP (GN173, SEQ ID NO: 2), with the 1-172 aa fragment of YFP (YN173, SEQ ID NO: 3), with the 1-172 aa fragment of CFP (CN173, SEQ ID NO: 4) and with the 1-172 aa fragment of BFP (BN173, SEQ ID NO: 5). The excitation/emission maxima for the corresponding heterologous fluorescent complexes were as follows: GN173-CC155-488/512 nm; YN173-CC155-503/515 nm; CN173-CC155-452/478 nm; BN173-CC155-384/450 nm (Hu C D, Kerppola T K. Simultaneous visualization of multiple protein interactions in living cells using multicolor fluorescence complementation analysis. Nat Biotechnol. 2003 May; 21(5):539-45). Thus, these split fluorescent proteins can be adopted to be used in the claimed methods. In a particular example, the CC155 fragment is fused to an immobilized polypeptide, and the GN173, YN173, CN173, BN173 fragments are fused to polypeptide-based binding agents. Methods of making protein fusions are well known in the art. Further, the binding agents fused to the GN173, YN173, CN173, BN173 fragments are used as a plurality of binding agents (as a mixture) that is contacting with an immobilized polypeptide fused to the CC155 fragment. Upon interaction of a binding agent from the plurality of binding agents with the immobilized polypeptide, a fluorescent detectable label is generated via interaction of the corresponding fluorescent protein fragments. Moreover, the signal generated by the detectable label is different for each binding agent from the plurality of binding agents, since emission spectra are different for the reconstituted fluorescent complexes (as shown in Hu C D, Kerppola T K, Nat Biotechnol. 2003 May; 21(5):539-45). Other variants of fluorescent proteins (such as red fluorescent protein) can potentially be split in a similar manner and fragments added to the mixture, extending the number of different generated detectable labels (reconstituted fluorescent complexes).

### Example 4. A Split Fluorescent Reporter

In this example, components of a split fluorescent reporter that is based on a small protein of 14 kDa (FAST) are used as first and second detection agents of the claimed methods. In a particular example, the N-terminal component of FAST (NFAST, SEQ ID NO: 6) is fused to an immobilized polypeptide, and the C-terminal component of FAST (CFAST, SEQ ID NO: 7) is fused to a polypeptide-based binding agent. Methods of making protein fusions are well known in the art. Upon interaction of the binding agent with the immobilized polypeptide, an interaction and complex formation between NFAST and CFAST occurs (as shown in Tebo et al., Nat Commun. (2019) 10(1):2822). This complex specifically and reversibly binds hydroxybenzylidene rhodanine (HBR) analogs displaying various spectral properties (Plamont, M.-A., et al., Small fluorescence-activating and absorption-shifting tag for tunable protein imaging in vivo. Proc. Natl Acad. Sci. USA (2016) 113, 497-502). Thus, reconstituted complex of NFAST and CFAST serves as a detectable label upon addition of a HBR analog to the reaction (it forms a fluorescent complex). The reconstituted complex of NFAST and CFAST, both fused to binding partners, shows affinity in the presence of HMBR (4-hydroxy-3-methylbenzylidene rhodanine, which provides green-yellow fluorescence) or HBR-3,5DOM (4-hydroxy-3,5-dimethoxybenzylidene rhodanine, which provides orange-red fluorescence), as shown in Tebo et al., Nat Commun. (2019) 10(1):2822. HBR analogs are weakly fluorescent in solution, but strongly fluoresce when immobilized in the binding cavity of FAST reconstituted from the NFAST and CFAST. This fluorogenic behavior provides high contrast even in the presence of an excess of fluorogenic chromophore.

### Example 5. Cyclic Decoding of Peptide

This example illustrate a decoding technique for identification of NTAAs through repeated cycles of binding pools of cognate binding agents (such as antibodies) combinatorially-labeled with the second detection agent. Repeated cycles generate a binary code representing the signal across the decoding cycles as disclosed in Gunderson et al. (“Decoding Randomly Ordered DNA Arrays,” Genome Research, 14:870-877, 2004).

In a first cycle of decoding, a subset of NTAAs on a plurality of peptides are detected in a “lighted” state by binding cognate binding agents having second detection agents (referred to as “labeled cognate antibodies” or “labeled Abs”). In this example, eight different labeled cognate antibodies are illustrated, referred to as “Ab1-Ab8.” Simultaneously, a subset of NTAAs on a plurality of peptide are detected in a “dark” state by binding cognate binding agents (such as antibodies) lacking the second detection agent (referred to as “unlabeled cognate antibodies”). Again, for purpose of this example, eight different unlabeled cognate are illustration, referred to as “Ab9-Ab16.”

FIG. 2A illustrates contacting the NTAAs of different peptides with both labeled and unlabeled antibodies, and further shows labeled antibody Ab 1 selectively binding the NTAA of the left-hand peptide (the “light” mode) and unlabeled antibody Ab9 selectively binding the NTAA of the right-hand peptide (the “dark” mode). FIG. 2B illustrates the corresponding light-dark decoding table for multiple decoding cycles. In the first cycle decoder pool, Ab1-Ab8 are labeled with a second detection agent. In the second cycle decoder pool, Ab1-Ab4 and Ab13-Ab16 are labeled with a second detection agent. The third and fourth cycle decoder pools are shown in FIG. 2B (by light and dark boxes). The “code” column of FIG. 2B represents the binary code extracted from the signal across the four decoding cycles. In this manner, the identity of the NTAAs may be determined by the digital code associated with each.

Sequence Listing
CC155 (155-238 aa portion of cyan fluorescent
protein)
SEQ ID NO: 1
DKQKNG IKANFKIRHN IEDGSVQLAD HYQQNTPIGD
GPVLLPDNHY LSTQSALSKD PKEKRDHMVL LEFVTAAGIT
HGMDELYK
GN173 (1-172 aa portion of green fluorescent
protein)
SEQ ID NO: 2
MSKGEELFT GVVPILVELD GDVNGHKFSV SGEGEGDATY
GKLTLKFICT TGKLPVPWPT LVTTLTYGVQ CFSRYPDHMK
QHDFFKSAMP EGYVQERTIF FKDDGNYKTR AEVKFEGDTL
VNRIELKGID FKEDGNILGH KLEYNYNSHN VYIMADKQKN
GIKVNFKIRH NIE
YN173 (1-172 aa portion of yellow fluorescent
protein)
SEQ ID NO: 3
MSKGEELFT GVVPILVELD GDVNGHKFSV SGEGEGDATY
GKLTLKFICT TGKLPVPWPT LVTTFGYGLQ CFARYPDHMK
QHDFFKSAMP EGYVQERTIF FKDDGNYKTR AEVKFEGDTL
VNRIELKGID FKEDGNILGH KLEYNYNSHN VYIMADKQKN
GIKVNFKIRH NIE
CN173 (1-172 aa portion of cyan fluorescent
protein)
SEQ ID NO: 4
MSKGEELFTG VVPILVELDG DVNGHKFSVS GEGEGDATYG
KLTLKFICTT GKLPVPWPTL VTTFSWGVQC FSRYPDHMKQ
HDFFKSAMPE GYVQERTIFF KDDGNYKTRA EVKFEGDTLV
NRIELKGIDF KEDGNILGHK LEYNYISHNV YITADKQKNG
IKANFKIRHN IE
BN173 (1-172 aa portion of blue fluorescent
protein)
SEQ ID NO: 5
MSKGEELFTG VVPILVELDG DVNGHKFSVS GEGEGDATYG
KLTLKFICTT GKLPVPWPTL VTTFSHGVQC FSRYPDHMKQ
HDFFKSAMPE GYVQERTIFF KDDGNYKTRA EVKFEGDTLV
NRIELKGIDF KEDGNILGHK LEYNFNSHNV YIMADKQKNG
IKVNFKIRHN IE
NFAST:
SEQ ID NO: 6
MEHVAFGSEDIENTLAKMDDGQLDGLAFGAIQLDGDGNILQYNAAEG
DITGRDPKQVIGKNFFKDVAPGTDSPEFYGKFKEGVASGNLNTMFEW
MIPTSRGPTKVKVHMKKALS
CFAST:
SEQ ID NO: 7
GDSYWVFVKRV

The present disclosure is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method for analyzing a polypeptide, comprising the steps of:
a. providing a polypeptide and an associated first detection agent attached to a solid support;
b. contacting the polypeptide with a binding agent capable of binding to the polypeptide, wherein the binding agent is joined to a second detection agent, whereby binding between the polypeptide and the binding agent brings the first detection agent and the second detection agent into sufficient proximity to interact with each other and generate a detectable label;
c. detecting a signal generated by the detectable label; and
d. repeating step (b) and step (c) sequentially one or more times.

2. The method of claim 1, wherein analyzing the polypeptide comprises identifying at least a portion of an amino acid sequence of the polypeptide.

3. The method of claim 1, wherein the first detection agent and the second detection agent, when brought into sufficient proximity, forms a detectable label precursor, and further comprising activating the detectable label precursor to form a detectable label.

4. The method of claim 3, wherein activating the detectable label precursor comprises binding an activating agent to a complex of the first detection agent and the second detection agent, wherein the activating agent is an allosteric activator of the first and/or second detection agent.

5. The method of claim 1, wherein generating the detectable label in step (b) comprises the second detection agent displacing a repressor protein or a blocking molecule from the first detection agent.

6. The method of claim 1, wherein the detectable label is selected from the group consisting of a bioluminescent label, a chemiluminescent label, a chromophore label, an enzymatic label, and a fluorescent label.

7. The method of claim 1, wherein the first detection agent is a first subunit of a split enzyme, the second detection agent is a second subunit of a split enzyme, and both the first detection agent and the second detection agent are enzymatically inactive.

8. The method of claim 7, wherein the first detection agent and the second detection agent comprise polypeptides.

9. The method of claim 7, wherein the first detection agent and the second detection agent comprise polynucleotides.

10. The method of claim 7, wherein the detectable label is an enzyme assembled from the first detection agent and the second detection agent interacting with each other, or a product of an enzymatic reaction catalyzed by the enzyme.

11. The method of claim 8, wherein the enzyme is a fluorescent protein.

12. The method of claim 1, wherein the first detection agent is associated with the polypeptide via a linker, wherein the linker is a tri-functional linker that comprises:
a. a moiety to associating with the polypeptide;
b. a moiety for associating with the support; and
c. a moiety for associating with the first detection agent.

13. The method of claim 1, wherein the first detection agent and the second detection agent do not comprise a polynucleotide, and do not undergo a polynucleotide-based hybridization or enzymatic covalent ligation to each other during generation of the detectable label.

14. The method of claim 1, wherein the detection in step (c) employs:
(a) a field effect transistor (FET) sensor;
(b) a chemical detection means;
(c) an optical detection means; or
(d) a detection of a change in pH.

15. The method of claim 1, wherein the detection in step (c) is a detection of fluorescence.

16. The method of claim 1, wherein the first detection agent and the second detection agent, when brought into sufficient proximity, are interacting through non-covalent interactions to form the detectable label.

17. The method of claim 1, wherein step (b) comprises contacting the polypeptide with a plurality of binding agents as a mixture; each binding agent is joined to a different second detection agent; and the signal generated by the detectable label is different for each binding agent.

18. The method of claim 1, further comprising: (d) removing a portion of the polypeptide, wherein step (d) is performed after step (c) and before repeating step (b), and wherein steps (b)-(d) are repeated sequentially one or more times.

19. The method of claim 18, wherein step (b) comprises contacting the polypeptide with a plurality of binding agents as a mixture; each binding agent is joined to a different second detection agent; and the signal generated by the detectable label is different for each binding agent.

20. The method of claim 18, wherein in each repetition during step (b) the polypeptide is contacted with a different binding agent that is joined to the same second detection agent.

21. The method of claim 18, wherein the portion of the polypeptide removed comprises the N-terminal amino acid (NTAA), thereby yielding a newly exposed NTAA of the polypeptide.

22. A method of identifying one or more binding events between a plurality of binding agents and a plurality of polypeptides, comprising: (a) providing a plurality of polypeptides attached to a solid support, wherein each polypeptide from the plurality of polypeptides is associated with a first detection agent; (b) contacting a polypeptide from the plurality of polypeptides with a plurality of binding agents, wherein at least one binding agent from the plurality of binding agents is capable of binding to the polypeptide, and wherein each binding agent from the plurality of binding agents is joined to a second detection agent, whereby binding between the polypeptide and the at least one binding agent brings the first detection agent and the second detection agent into sufficient proximity to interact with each other and generate a detectable label; (c) detecting a signal generated by the detectable label, thereby identifying the binding between the polypeptide and the at least one binding agent; (d) optionally, removing a portion of the polypeptide; and
repeating steps (b), (c) and (d) sequentially one or more times.


SEQUENCE LISTING (7 sequences)

SEQ ID NO: 1 - protein, 84 aa, Artificial Sequence
  CC155 (155-238 aa portion of cyan fluorescent protein)
DKQKNGIKANFKIRHNIEDGSVQLADHYQQNTPIGDGPVLLPDNHYLSTQSALSKDPKEKRDHMVLLEFVTAAGITHGMDELYK

SEQ ID NO: 2 - protein, 172 aa, Artificial Sequence
  GN173 (1-172 aa portion of green fluorescent protein)
MSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIE

SEQ ID NO: 3 - protein, 172 aa, Artificial Sequence
  YN173 (1-172 aa portion of yellow fluorescent protein)
MSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIE

SEQ ID NO: 4 - protein, 172 aa, Artificial Sequence
  CN173 (1-172 aa portion of cyan fluorescent protein)
MSKGEELFTGVVPILVELDGDVNGHKFSVSGEGEGDATYGKLTLKFICTTGKLPVPWPTLVTTFSWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIE

SEQ ID NO: 5 - protein, 172 aa, Artificial Sequence
  BN173 (1-172 aa portion of blue fluorescent protein)
MSKGEELFTGVVPILVELDGDVNGHKFSVSGEGEGDATYGKLTLKFICTTGKLPVPWPTLVTTFSHGVQCFSRYPDHMKQHDFFKSAMPEGYVQERTIFFKDDGNYKTRAEVKFEGDTLVNRIELKGIDFKEDGNILGHKLEYNFNSHNVYIMADKQKNGIKVNFKIRHNIE

SEQ ID NO: 6 - protein, 114 aa, Artificial Sequence
  NFAST
MEHVAFGSEDIENTLAKMDDGQLDGLAFGAIQLDGDGNILQYNAAEGDITGRDPKQVIGKNFFKDVAPGTDSPEFYGKFKEGVASGNLNTMFEWMIPTSRGPTKVKVHMKKALS

SEQ ID NO: 7 - protein, 11 aa, Artificial Sequence
  CFAST
GDSYWVFVKRV